SEQUENCE LISTING (16 sequences)

SEQ ID NO: 1 - protein, 638 aa, synthetic construct
MNRGVPFRHLLLVLQLALLPAATQGYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNEDVDHHHHHHGGGGSGGGGSGGGGSKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 2 - protein, 25 aa, synthetic construct
MNRGVPFRHLLLVLQLALLPAATQG

SEQ ID NO: 3 - protein, 159 aa, synthetic construct
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNEDVD

SEQ ID NO: 4 - protein, 6 aa, synthetic construct
HHHHHH

SEQ ID NO: 5 - protein, 15 aa, synthetic construct
GGGGSGGGGSGGGGS

SEQ ID NO: 6 - protein, 433 aa, synthetic construct
KKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 7 - DNA, 1917 bp, synthetic construct
  source 1..1917: mol_type = other DNA
atgaatcgaggggtacccttcaggcatctgttgctcgttctgcagctcgcccttctccctgcggccactcagggctactttgggaagttggagtctaagctcagcgtgattagaaacctcaatgaccaggttctgtttatcgatcagggaaatcggcccctgtttgaagatatgactgatagcgattgtagggataacgcgcccagaaccatattcattatctcaatgtataaggacagccagcctagaggtatggcggtgacaatatccgtgaagtgcgaaaagatctccacgcttagttgtgagaataagattataagcttcaaagaaatgaatccgccagacaatattaaagacacgaaatcagacataatcttttttcaacgatctgtcccgggccatgacaacaaaatgcagttcgaaagctcaagttacgagggctattttctcgcgtgcgaaaaggaaagagatcttttcaaactgattctgaagaaggaggatgagctcggcgacaggtctatcatgtttaccgtccaaaatgaggatgtagatcatcaccaccaccatcacgggggtggtggaagtggtggcgggggatccgggggtgggggatccaagaaagtggtactgggcaagaagggagatacagttgagttgacatgtacagcatcccaaaagaaaagcattcagtttcactggaagaactctaaccaaatcaagattctgggaaatcaaggatcattccttactaaggggcccagcaaactgaacgaccgcgccgattcacgacggtctttgtgggatcagggcaatttccccctcataattaagaacctcaagattgaggactcagacacctacatatgtgaagtcgaggatcagaaggaagaggtacagctcctggtatttggtttgaccgcaaattccgacacccacctgctccaagggcagagcctcacactcacactggaatcaccgcctggctccagtccctccgtacaatgccggtcccctcgaggtaaaaatattcaagggggaaagacactgtctgtctcccagcttgagttgcaagacagcggtacgtggacatgcaccgtactgcagaaccagaagaaggtggaatttaagatagatatagtcgtacttgcgtttcagaaagcgagcagtattgtatataagaaggaaggtgagcaagtcgaattcagtttcccgctggctttcacggtagaaaaactgacagggagtggcgaattgtggtggcaggcggagcgcgcatctagtagtaaaagttggataaccttcgaccttaaaaataaagaggtaagtgtgaagcgcgtaactcaagacccgaagcttcagatggggaagaaattgcctttgcatctgaccctgccacaagcgctgcctcaatacgcgggtagtgggaaccttacgcttgcacttgaggcaaaaactggcaaactgcaccaggaggtgaatctggttgttatgagagcaacgcagctccagaagaatctcacgtgcgaagtttggggtccaacttcacctaagctcatgttgagtcttaaactcgaaaacaaggaagccaaagtctctaagcgagaaaaggccgtctgggtgctgaatcctgaagcaggaatgtggcagtgcctcctctcagatagtggacaagtacttctcgagagtaacataaaggtattgccgacgtggagcacccccgtgcagcctatggcgttgatcgtactggggggtgttgccggtctcctgctctttatcggacttggtatctttttttgtgtacgatgtagacatagaaggaggcaagcagaacgaatgagccaaattaagaggctcctgagcgagaagaaaacttgccaatgtcctcacaggtttcaaaagacgtgctcccccatataa

SEQ ID NO: 8 - DNA, 75 bp, synthetic construct
  source 1..75: mol_type = other DNA
atgaatcgaggggtacccttcaggcatctgttgctcgttctgcagctcgcccttctccctgcggccactcagggc

SEQ ID NO: 9 - DNA, 476 bp, synthetic construct
  source 1..476: mol_type = other DNA
actttgggaagttggagtctaagctcagcgtgattagaaacctcaatgaccaggttctgtttatcgatcagggaaatcggcccctgtttgaagatatgactgatagcgattgtagggataacgcgcccagaaccatattcattatctcaatgtataaggacagccagcctagaggtatggcggtgacaatatccgtgaagtgcgaaaagatctccacgcttagttgtgagaataagattataagcttcaaagaaatgaatccgccagacaatattaaagacacgaaatcagacataatcttttttcaacgatctgtcccgggccatgacaacaaaatgcagttcgaaagctcaagttacgagggctattttctcgcgtgcgaaaaggaaagagatcttttcaaactgattctgaagaaggaggatgagctcggcgacaggtctatcatgtttaccgtccaaaatgaggatgtagat

SEQ ID NO: 10 - DNA, 18 bp, synthetic construct
  source 1..18: mol_type = other DNA
catcaccaccaccatcac

SEQ ID NO: 11 - DNA, 45 bp, synthetic construct
  source 1..45: mol_type = other DNA
gggggtggtggaagtggtggcgggggatccgggggtgggggatcc

SEQ ID NO: 12 - DNA, 1302 bp, synthetic construct
  source 1..1302: mol_type = other DNA
aagaaagtggtactgggcaagaagggagatacagttgagttgacatgtacagcatcccaaaagaaaagcattcagtttcactggaagaactctaaccaaatcaagattctgggaaatcaaggatcattccttactaaggggcccagcaaactgaacgaccgcgccgattcacgacggtctttgtgggatcagggcaatttccccctcataattaagaacctcaagattgaggactcagacacctacatatgtgaagtcgaggatcagaaggaagaggtacagctcctggtatttggtttgaccgcaaattccgacacccacctgctccaagggcagagcctcacactcacactggaatcaccgcctggctccagtccctccgtacaatgccggtcccctcgaggtaaaaatattcaagggggaaagacactgtctgtctcccagcttgagttgcaagacagcggtacgtggacatgcaccgtactgcagaaccagaagaaggtggaatttaagatagatatagtcgtacttgcgtttcagaaagcgagcagtattgtatataagaaggaaggtgagcaagtcgaattcagtttcccgctggctttcacggtagaaaaactgacagggagtggcgaattgtggtggcaggcggagcgcgcatctagtagtaaaagttggataaccttcgaccttaaaaataaagaggtaagtgtgaagcgcgtaactcaagacccgaagcttcagatggggaagaaattgcctttgcatctgaccctgccacaagcgctgcctcaatacgcgggtagtgggaaccttacgcttgcacttgaggcaaaaactggcaaactgcaccaggaggtgaatctggttgttatgagagcaacgcagctccagaagaatctcacgtgcgaagtttggggtccaacttcacctaagctcatgttgagtcttaaactcgaaaacaaggaagccaaagtctctaagcgagaaaaggccgtctgggtgctgaatcctgaagcaggaatgtggcagtgcctcctctcagatagtggacaagtacttctcgagagtaacataaaggtattgccgacgtggagcacccccgtgcagcctatggcgttgatcgtactggggggtgttgccggtctcctgctctttatcggacttggtatctttttttgtgtacgatgtagacatagaaggaggcaagcagaacgaatgagccaaattaagaggctcctgagcgagaagaaaacttgccaatgtcctcacaggtttcaaaagacgtgctcccccatataa

SEQ ID NO: 13 - protein, 632 aa, synthetic construct
MNRGVPFRHLLLVLQLALLPAATQGYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNEDVDGGGGSGGGGSGGGGSKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 14 - protein, 5 aa, synthetic construct
  REPEAT 1..5: note = repeat n times, where n is an integer of at least one
GSGGS

SEQ ID NO: 15 - protein, 5 aa, synthetic construct
  REPEAT 1..5: note = repeat n times, where n is an integer of at least one
GGGGS

SEQ ID NO: 16 - protein, 4 aa, synthetic construct
  REPEAT 1..4: note = repeat n times, where n is an integer of at least one
GGGS

(19) United States
(12) Patent Application Publication
(10) Pub. No.: US 2026/0265324 A1
(43) Pub. Date: Sep. 10, 2026

(54) INTERLEUKIN-18 PRETREATMENT AS A CRYOPROTECTANT FOR LYMPHOCYTES

(71) Applicant: The Trustees of the University of Pennsylvania, Philadelphia, PA (US)

(72) Inventors: Neil Sheppard, Philadelphia, PA (US); Abdulla Berjis, Philadelphia, PA (US)

(21) Appl. No.: 19/469,536

(22) PCT Filed: Mar. 29, 2024
(86) PCT No.: PCT/US2024/022389

§ 371 Date: Sep. 26, 2025

Related U.S. Application Data
(60) Provisional application No. 63/493,096, filed on Mar. 30, 2023.

(51) Int. Cl. C07K 14/54; A01N 1/162; C07K 14/705

(52) U.S. Cl. CPC C07K 14/54; A01N 1/162; C07K 14/70514

(57) ABSTRACT

The present invention includes methods useful for cryopreserving immune cells, including natural killer cells (NK cells) and T cells comprising pre-treating the cells with IL-18 and IL-15 cytokine. Also provided is an IL-18/CD4 fusion protein. In some embodiments, the invention includes compositions comprising said pre-treated, cryopreserved cells.

### CROSS-REFERENCE TO RELATED APPLICATION

The present application is entitled to priority under 35 U.S.C. § 119(e) to U.S. Provisional Patent Application No. 63/493,096, filed Mar. 30, 2023, which is hereby incorporated herein by reference in its entirety.

### BACKGROUND OF THE INVENTION

Poor recovery from cryopreservation is somewhat overlooked issue in T cell therapies such as CAR-T therapies and is a significant barrier to successful commercial development of natural killer (NK) cell-based therapies due to the poor recovery of these cells following thawing. Current solutions to poor NK yield often consist of little more than increasing the number of injected cells. Recent studies, however, have demonstrated that NK cells which survive cryopreservation often have significant functional deficits that limit their cytotoxic function and clinical utility.

Thus, there is a need for cryopreservation techniques for lymphocytes in general, and NK cells in particular, which not only maintain high levels of viability post-thawing, but also preserve clinically relevant cellular functions. The present invention addresses this need.

### SUMMARY OF THE INVENTION

As described herein, the present invention relates to methods useful for cryopreserving immune cells, including natural killer cells (NK cells) and T cells comprising pre-treating the cells with IL-18 and IL-15 cytokine. Also provided is an IL-18/CD4 fusion protein. In some embodiments, the invention includes compositions comprising said pre-treated, cryopreserved cells.

In one aspect, the current invention includes a method for cryopreserving an isolated immune cell or precursor cell thereof, said method comprising:
a. pre-treating the isolated immune cell or precursor cell thereof for at least 24 hours with IL-18 at a concentration between 1.0 and 500 ng/ml;
d. resuspending the pre-treated immune cell or precursor cell thereof in a cryoprotective medium and freezing the cell at −80° C.; and
e. storing the cryopreserved cell in liquid nitrogen (N2);
wherein the method maintains viability and cytotoxic function of the cryopreserved immune cell or precursor cell thereof after thawing.

In certain embodiments, the isolated immune cell or precursor cell thereof is cultured or expanded for at least 7 days prior to cryopreservation.

In certain embodiments, the method of the above aspect or any aspect or embodiment disclosed herein further comprises pre-treating the expanded immune cells or precursors thereof with an additional cytokine.

In certain embodiments, the additional cytokine is IL-15, wherein the IL-15 is used at a concentration between 1.0 and 500 ng/ml.

In certain embodiments, the immune cell or precursor thereof is a natural killer (NK) cell or NK cell precursor cell.

In certain embodiments, the NK cell is isolated from a source selected from the group consisting of a patient, a healthy adult donor, cord blood, CD34+ cord blood stem cells, an induced pluripotent stem cell (iPSC), and an NK cell line.

In certain embodiments, the immune cell is a T cell or T cell precursor cell.

In certain embodiments, the subject is a mammal.

In certain embodiments, the subject is a human.

In another aspect, the current invention includes a fusion protein comprising a first protein domain and a second protein domain separated by a linker, wherein the first protein domain comprises IL-18 peptide and the second protein domain comprises a human CD4 receptor.

In certain embodiments, the IL-18 peptide is a mature IL-18 peptide.

In certain embodiments, the IL-18 peptide comprises the amino acid sequence set forth in SEQ ID NO: 3.

In certain embodiments, the CD4 receptor domain comprises the amino acid sequence set forth in SEQ ID NO: 6.

In certain embodiments, the fusion protein of the above aspects or embodiments or any aspect or embodiment disclosed herein further comprises a CD4 signal peptide.

In certain embodiments, the CD4 signal peptide comprises the amino acid sequence set forth in SEQ IN NO: 2.

In certain embodiments, the linker is a G4S linker.

In certain embodiments, the linker comprises the amino acid sequence set forth in SEQ ID NO: 5.

In certain embodiments, the fusion protein comprises the amino acid sequence set forth in SEQ ID NOs: 1 or 13.

In another aspect, the current invention includes an isolated nucleic acid encoding a fusion protein comprising a first protein domain and a second protein domain separated by a linker, wherein the first protein domain comprises IL-18 peptide and the second protein domain comprises a human CD4 receptor.

In certain embodiments, the IL-18 peptide is a mature IL-18 peptide.

In certain embodiments, the IL-18 peptide is encoded by a nucleic acid sequence set forth in SEQ ID NO: 9.

In certain embodiments, the CD4 receptor domain is encoded by a nucleic acid sequence set forth in SEQ ID NO: 12.

In certain embodiments, the isolated nucleic acid of the above aspects or embodiments or any aspect or embodiment disclosed herein further comprises a nucleic acid sequence encoding a CD4 signal peptide.

In certain embodiments, the nucleic acid sequence encoding a CD4 signal peptide comprises the nucleic acid sequence set forth in SEQ IN NO: 8.

In certain embodiments, the linker is a G4S linker.

In certain embodiments, the linker is encoded by a nucleic acid sequence set forth in SEQ ID NO: 11.

In certain embodiments, the fusion protein is encoded by a nucleic acid sequence set forth in SEQ ID NOs: 1 or 13.

In another aspect, the current invention includes a vector comprising the isolated nucleic acid of any one of the above aspects or embodiments or any aspect or embodiment disclosed herein.

In certain embodiments, the vector is an expression vector.

In certain embodiments, the vector is selected from the group consisting of a DNA vector, an RNA vector, a plasmid, a lentiviral vector, an adenoviral vector, an adeno-associated vector, and a retroviral vector.

In another aspect, the current invention includes a host cell comprising the isolated nucleic acid of any one of the above aspects or embodiments or any aspect or embodiment disclosed herein or the vector of any one of the above aspects or embodiments or any aspect or embodiment disclosed herein.

In certain embodiments, the host cell is of eukaryotic origin.

In certain embodiments, the host cell is of mammalian origin.

In certain embodiments, the host cell is selected from the group consisting of a K562 cell, a HEK293, and an EBV-transformed cell.

In another aspect, the current invention includes a method for producing a composition comprising an isolated immune cell or precursor cell thereof, said method comprising:
a. pre-treating the isolated immune cell or precursor cell thereof for at least 24 hours with IL-18 at a concentration between 1.0 and 500 ng/ml;
d. resuspending the pre-treated immune cell or precursor cell thereof in a cryoprotective medium and freezing the cell at −80° C.;
e. storing the cryopreserved cell in liquid nitrogen (N2); and
f. thawing cryopreserved cell and resuspending it in a pharmaceutically acceptable excipient, carrier, or diluent.

In certain embodiments, the method further comprises culturing or expanding the isolated immune cell or precursor thereof for at least 7 days.

In certain embodiments, the method of the above aspect or embodiment or any aspect or embodiment disclosed herein further comprises pre-treating the expanded immune cells or precursors thereof with an additional cytokine.

In certain embodiments, the additional cytokine is IL-15, wherein the IL-15 is used at a concentration between 1.0 and 500 ng/ml.

In certain embodiments, the immune cell or precursor thereof is a natural killer (NK) cell or NK cell precursor cell.

In certain embodiments, the NK cell is isolated from a source selected from the group consisting of a patient, a healthy adult donor, cord blood, CD34+ cord blood stem cells, an induced pluripotent stem cell (iPSC), and an NK cell line.

In certain embodiments, the immune cell is a T cell or T cell precursor cell.

In certain embodiments, the subject is a mammal.

In certain embodiments, the subject is a human.

In another aspect, the current invention includes a method for producing a composition comprising an isolated immune cell or precursor cell thereof, said method comprising:
a. co-culturing an isolated immune cell or precursor cell thereof with a composition comprising an IL-18-CD4 fusion protein;
d. resuspending the pre-treated immune cell or precursor cell thereof in a cryoprotective medium and freezing the cell at −80° C.;
e. storing the cryopreserved cell in liquid nitrogen (N2); and
f. thawing cryopreserved cell and resuspending it in a pharmaceutically acceptable excipient, carrier, or diluent.

In certain embodiments, the fusion protein comprises a first protein domain and a second protein domain separated by a linker, wherein the first protein domain comprises IL-18 peptide and the second protein domain comprises a human CD4 receptor.

In certain embodiments, the IL-18 peptide comprises the amino acid sequence set forth in SEQ ID NO: 3.

In certain embodiments, the CD4 receptor domain comprises the amino acid sequence set forth in SEQ ID NO: 6.

In certain embodiments, the fusion protein further comprises a CD4 signal peptide.

In certain embodiments, the CD4 signal peptide comprises the amino acid sequence set forth in SEQ IN NO: 2.

In certain embodiments, the linker is a G4S linker.

In certain embodiments, the linker comprises the amino acid sequence set forth in SEQ ID NO: 5.

In certain embodiments, the fusion protein comprises the amino acid sequence set forth in SEQ ID NOs: 1 or 13.

In certain embodiments, the method of the above aspect or embodiment or any aspect or embodiment disclosed herein further comprises pre-treating the expanded immune cells or precursors thereof with an additional cytokine.

In certain embodiments, the additional cytokine is IL-15, wherein the IL-15 is used at a concentration between 1.0 and 500 ng/ml.

In certain embodiments, the immune cell or precursor thereof is a natural killer (NK) cell or NK cell precursor cell.

In certain embodiments, the immune cell is a T cell or T cell precursor cell.

In certain embodiments, the subject is a mammal.

In certain embodiments, the subject is a human.

In another aspect, the current invention includes a method for treating a disease in a subject in need thereof, comprising administering to the subject an effective amount of the composition of any one of claims 33-54, thereby treating the disease.

In certain embodiments, the disease is a cancer.

In certain embodiments, the method of the above aspect or embodiment or any aspect or embodiment disclosed herein further comprises administering to the subject an additional therapeutic agent.

In certain embodiments, the additional therapeutic agent is a cytokine.

In certain embodiments, the cytokine is IL-15.

### BRIEF DESCRIPTION OF THE DRAWINGS

The following detailed description of specific embodiments of the invention will be better understood when read in conjunction with the appended drawings. For the purpose of illustrating the invention, there are shown in the drawings exemplary embodiments. It should be understood, however, that the invention is not limited to the precise arrangements and instrumentalities of the embodiments shown in the drawings.

FIG. 1 depicts NK cell recovery after thawing. NK cells were cryopreserved using CryoStor® CS5 using a controlled rate freezer chamber in −80° C. freezer. After 10-14 hours cells were moved to liquid nitrogen freezer. Cells were thawed in 37° C. water bath until small piece of ice was left then, suspended in NK media (Lonza X-vivo 10, 10% Human AB serum, 100 U/mL IL-2) then counted by flow cytometry. Significant cell loss after is seen by 24 h post thaw, after which the cells start to proliferate and numbers recover, though still below 50% of the amount cryopreserved even after 72 h culture with IL-2.

FIG. 2 illustrates cytotoxicity of thawed NK cells. NK cells were thawed and rested in NK medium for 24 hours prior to use in a cytotoxicity assay using Raji b2M KO cells transduced with click beetle green (CBG). Significant loss in NK cell cytotoxicity is seen after thawing.

FIG. 3 illustrates cytokine enhancement of NK survival. NK cells were treated with IL-12 100 ng/ml, IL-15 50 ng/ml, IL-18 200 ng/ml, IL-12 100 ng/ml+IL-15 50 ng/ml, IL-12 100 ng/ml+IL-18 200 ng/ml, IL-15 50 ng/ml+IL-18 200 ng/ml, IL-18 200 ng/ml+IL-12 100 ng/ml or IL-12 100 ng/ml+IL-18 200 ng/ml+IL-15 50 ng/ml for 24 hours prior to cryopreservation in CS5. Cells were thawed and live cell number was measured. IL-18+IL-15 significantly improved NK cells recovery after cryopreservation. Although IL-18 alone also improved NK cell recovery after cryopreservation. A trend that IL-15+IL-18 is better was observed. (Unpaired t-test p=0.0009 for untreated versus the combination of IL-15+IL-18).

FIG. 4 illustrates that no significant loss in NK cell cytotoxicity is seen after thawing. IL-15+IL-18 treated NK cell were thawed and rested for 24 hours then a killing assay using CBG expressing B2M KO Raji cells was performed.

FIG. 5 illustrates that NK cell recovery improved further with greater concentrations of IL-18, although with diminishing returns in terms of additional recovery for each doubling of IL-18 concentration. To determine the optimal dose of IL-18, NK cells were stimulated with a 2-fold dilution series of IL-18 from 500 ng/ml to 1.9 ng/ml prior to cryopreservation. After thawing, NK viability was assayed, and it was found that an IL-18 concentration 125 ng/ml was required to enable a high recovery rate.

FIG. 6 illustrates that IL-15 potentiates the post cryopreservation viability benefit of IL-18 but does not have a benefit by itself. To determine the optimal dose of IL-15, NK cells were stimulated with a 2-fold dilution series of IL-15 from 500 ng/ml to 1.9 ng/ml prior to cryopreservation. After thawing, NK viability was assayed, and it was found that IL-15 concentrations between 1.9 and 500 ng/mL did not impact NK cell viability in the absence of IL-18.

FIG. 7 illustrates that a beneficial effect with >50% recovery was seen after as little as 4 h, but the maximal effect was not observed until 24 h suggesting it takes between 12 and 24 h for the combined effects of IL-15+IL-18 to occur. NK cells were stimulated with IL-15 and IL-18 for 0, 4, 8, 12 or 24 hours then cryopreserved using CryoStor CS5. Cells were thawed and live NK cells were counted using flow cytometry.

FIG. 8 is a diagram showing the structure of membrane bound mature IL-18 (CD4-IL-18). Human CD4 transmembrane extracellular domain was used. Mature IL-18 was tag with 6His tag then attached to CD4 with a Flexible linker 3(G4S). As control construct was made without the IL-18.

FIG. 9 illustrates the amino acid sequence of the CD4-IL-18 construct. Blue=CD4 signal peptide, Red=mature form of IL-18, Black=6His tag, Green=G4S linker and yellow CD4 receptor.

FIG. 10 illustrates the nucleotide sequence of the CD4-IL-18. Blue=CD4 signal peptide, Red=mature form of IL-18, Black=6His tag, Green=G4S linker and yellow CD4 receptor.

FIG. 11 illustrates that a significant increase in SEAP was seen with CD4-IL-18 but not with CD4 plasmid demonstrating that the CD4-IL-18 construct successfully activates the IL-18 pathway. To test CD4-IL-18 construct IL-18 Reporter HEK 293 Cells were used. IL-18 cells HEK cells were electroplated with CD4 or CD4-IL-18 plasmids.

FIG. 12 illustrates expression of CD4 and CD4-IL-18 protein on K562 cells. K562 cells were electroplated with CD4 or CD4-IL-18 plasmids. Surface expression was measured using anti-human CD4 antibody.

FIG. 13 illustrates expression of CD4 and CD4-IL-18 protein on K562 cells. K562 cells were electroplated with CD4 or CD4-IL-18 plasmids. Surface expression was measured using anti-human IL-18 antibody.

FIG. 14 illustrates that NK cells cultured with K562 cells expressing CD4-IL-18 co-cultured NK had a better recovery after thawing at 24 hours' time point. NK cells were co-cultured with irradiated K562 cells expressing either CD4 or membrane bound CD-IL-18 24 hours before cryopreservation.

FIG. 15 illustrates a diagram showing NK cell mouse model. NSG mice were inject with 0.75e6 B2M KO CBG positive Raji cells I.V (tail injection) per mouse. Seven days after tumor injection mice were inject with 20×10⁶ NK cells per mouse. Mice were injected with 5 ug per mouse IL-15 three days after NK injection. Mice were then imaged every 3 days starting at day 0 after NK cell injection. Mice were bled at day 7 after NK injection.

FIG. 16 illustrates the results of an in vivo mouse tumor model using the Raji lymphoma cell line. Tumor bearing mice were then treated with an adoptive transfer of fresh or cryopreserved NK cells. Total flux was measured at day 0, 3, 7 and 10 after NK injection. NK cells treated with IL-15+IL-18 controlled the tumor significantly better than untreated cells. one-way ANOVA p=0.0001 at day 3 and 7 after NK cell injection.

FIG. 17 illustrates that mice treated with IL-15+IL-18 NK cells had significantly more NK cells in the blood. Mice implanted with Raji cell line tumors and treated with an adoptive transfer of fresh and cryopreserved NK cells were bled through cheek vein and NK cells count in the blood was measured using Trucount™ tube (BD) flow cytometry assays. Unpaired t-test p<0.0001

FIG. 18 illustrates the survival curve for mice in the Raji tumor model study. Mice treated with IL-15+IL-18 NK cells survived longer than mice treated with untreated NK cells.

FIG. 19 is a diagram of the workflow for NK cell cryopreservation.

FIG. 20 illustrates flow cytometry gating of live NK cells.

FIG. 21 is a graph illustrating that there is no significant loss of NK cells immediately following thawing.

FIG. 22 illustrates that NK cells die by apoptosis in the first 24 h post-thaw, with half-maximal caspase activation by 5-6 hours post-thaw.

FIG. 23 illustrates cytokine-treated NK cell recovery after thaw.

FIG. 24 illustrates that IL-18 pre-treatment shows a trend towards reduced caspase-3/7 activation upon thaw.

FIGS. 25A-25D illustrates that IL-18 pre-treatment prevents the loss of cytotoxicity against K562 target cells upon thaw among surviving NK cells. FIG. 25A. Treatment with IL-18. FIG. 25B. Treatment with IL-15. FIG. 25C. Treatment with IL-12. FIG. 25D. Graphical representation of the results of the individual cytokine treatment studies.

FIG. 26 illustrates that IL-18 Pre-Treatment also prevents loss of T cell viability post thaw.

FIGS. 27A-27I illustrate the characterization of NK cell recovery, phenotype, and function after cryopreservation. FIG. 27A. NK cell viability after thawing assessed via flow cytometry (n=5 healthy donors). FIG. 27B. NK cell recovery 24, 48 and 72 hours after thawing in comparison to starting NK cell number at time 0 hours after thawing (n=10 healthy donors). FIG. 27C. CD107A degranulation assay before and after thawing. NK cells were cultured with K562 cells at 1:1 effector:Target ratio for 3 hours (n=4 healthy donors). FIG. 27D. NK cytotoxicity assay before and after cryopreservation with B2M KO Raji cells (n=4 healthy donors). FIG. 27E. NK cytotoxicity assay before and after cryopreservation with K562 cells (n=4 healthy donors). FIG. 27F ADCC assay using anti-CD20 antibody and Jeko-1 cells (n=3 healthy donors). FIG. 27G Single cell secretome profile of cryopreserved and non-cryopreserved NK cells treated with R848 for 24 h.). Heatmap showing individual cytokines that NK cell secreted (n=3 healthy donors). FIG. 27H Bulk RNA volcano plot comparing before, 24 and 72 hours after cryopreservation (n=5 healthy donors). FIG. 27I GSEA of IL-2-STAT5 pathway. Normality test was used to determine the distribution of the data then parametric test t test was used for FIG. 27A and FIG. 27C. Two-way RM ANOVA was used for FIG. 27D, FIG. 27E and FIG. 27F. The Benjamini-Hochberg method was used to obtain p value shown in FIG. 27H. Two-tailed test was used for FIGS. 27A, 27C and 27H. FIG. 27G error bars are shown as mean SD. All other graphs are shown as mean±SEM.

FIGS. 28A-28I illustrate that GZMB leakage induces apoptosis in NK cells after cryopreservation FIG. 28A. NK cell recovery immediately before and after cryopreservation (n=4 healthy donors). FIG. 28B. IncucyteCR live imagining using caspase 3/7 cleavable dye to indicate early apoptosis (n=4 healthy donors). FIG. 28C. Incucyte® live imagining using CYTOX™ NIR dye to indicate loss of plasma membrane integrity (n=4 healthy donors. FIG. 28D. granzyme B levels at time 0 and 24 h after thawing (n=5 healthy donors). FIG. 28E. GZMB knock-out NK cells recovery after cryopreservation (n=4 healthy donors). FIG. 28F. Perform knock-out NK cells recovery after cryopreservation (n=4 healthy donors). FIG. 28G. GZMB and mock KO NK cells were cocultured at 1 ratio of 1:10 in both directions to determine role of fratricide in NK cell death (n=4 healthy donors). FIGS. 28H, and 28I Confocal microscopy imaging showing extent of colocalization of GZMB and LAMP-3, a marker for cytotoxic granules in fresh versus cryopreserved NK cells (fresh n=29, cryopreserved n=42). Normality test was used to determine the distribution of the data then parametric test t test was used for FIGS. 28A, 28D, 28E, 28F, 28G. non-parametric Mann Whitney test was used for FIG. 28H. Two-tailed test was used for FIGS. 28A, 28D, 28E, 28F, 28G, and 28H. All graphs are shown as mean±SEM.

FIGS. 29A-29M illustrate that IL-15+IL-18 pretreatment improves NK cells recovery and function after cryopreservation by inducing GZMB release and upregulating anti-apoptotic genes. FIGS. 29A and 29B. Pretreating NK cells with cytokines 24 h before cryopreservation (n=10 healthy donors FIG. 29A and n=6 healthy donors for FIG. 29B). FIG. 29C. GZMB MFI 24 hours after vehicle or IL-12 treatment (n=7 healthy donors). FIG. 29D. NK cell-mediated killing assay using IL-15+IL-18-pretreated NK cell before and after cryopreservation (n=4 healthy donors). FIG. 29E. Single cell secretome profile of IL-15+IL-18 treated cryopreserved and fresh NK cells treated with R848 for 24 h (n=3 healthy donors). FIG. 29F. CD107A degranulation after IL-15+IL-18 treatment (n=6 healthy donors). FIG. 29G. Granzyme B ELISA 24 h after IL-15+IL-18 treatment (n=6 healthy donors). FIG. 29H. Comparing MFI of IL-15+IL-18-treated NK cells at 24- and 72-h post-treatment (n=7 healthy donors). FIG. 29I. Volcano plot comparing IL-15+IL-18-treated vs untreated NK cells (n=3 healthy donors). FIG. 29J. Heatmap showing NA levels using NanoString assay (n=3 healthy donors). FIG. 29K. MFI quantification of BCL2L1 after IL-15+IL-18 treatment (n=3 healthy donors). FIG. 29L Recovery of perform KO and BCL2L1 KO NK cells with and without IL-15+IL18 pretreatment (n=3 healthy donors). FIG. 29M. IL-15+IL18-treated NK cell cryopreservation using GMP control rate freezer (n=4 healthy donors). Normality test was used to determine the distribution of the data then parametric test t test was used for FIGS. 29C. 29G, 29K, 29L, 29M, non-parametric Wilcoxon matched pairs t test was used for FIGS. 29A, 29B, 29F and 29H Two-way RM ANOVA was used for FIG. 29D. The Benjamini-Hochberg method was used to obtain p value shown in FIGS. 291 and 29J. Two-tailed test was used for FIGS. 29A, 29B, 29C, 29F, 229G, 29H, 29I, 29J, 29K. 29L, and 29M. FIG. 29E. error bars are shown as mean SD. All other graphs are shown as mean±SEM.

FIGS. 30A-30D illustrate deep immuno-profiling of vehicle and IL-15+18 treated NK cells after cryopreservation using cytometry by time of flight (CyTOF) reviles distinct changes in NK cells phenotypes. FIG. 30A. FlowSOM cluster map (n=3 healthy donors). FIG. 30B. individual marker expression in UMAP-1 (n=3 healthy donors). FIG. 30C. heatmap showing markers and expression levels in different clusters (n=3 healthy donors). FIG. 30D. UMAP showing NK cells distribution at different time points after thawing (n=3 healthy donors).

FIGS. 31A-E illustrate that IL-15+IL-18 pretreatment enables equipotent in vivo activity of cryopreserved and non-cryopreserved untreated NK cells. FIG. 31A. Schematic diagram for in vivo experiment. FIG. 31B quantification of tumor burden (flux) (n=9 mice/group). FIG. 31C NK cell number in the blood of treated mice (n=9 mice/group). FIG. 31D Survival curve of NK treated mice (n=9 mice/group). Two-way ANOVA was used for (FIG. 31B). Normality test was used to determine the distribution of the data then parametric test t test was used for FIG. 31C. Simple survival analysis (Kaplan-Meier) with no data adjustment were made for FIG. 31D. Two-tailed test was used for FIG. 31D. All graphs are shown as mean±SEM.

FIGS. 32A-32E illustrate a characterization of NK cell function and gene expression after cryopreservation. FIG. 32A. Flow cytometry gating strategy used for all recovery and viability graphs. FIG. 32B Sorting strategy for Raji B2M KO cells. FIG. 32C Confirmation of B2M KO in Raji cells via flow cytometry. FIG. 32D CD20 expression on Jeko-1 cell line used as a target for ADCC with anti-CD20 mAb. FIG. 32E. NK cell recovery after cryopreservation using published protocol (n=4 healthy donors). Normality test was used to determine the distribution of the data, then two tailed t test was used for d. all graphs are shown as mean±SEM.

FIGS. 33A-33B illustrate validation of GZMB and perforin KO. FIG. 33A. DNA % indels and flow cytometry showing GZMB KO efficiency (n=4 healthy donors). FIG. 33B. DNA indels and flow cytometry showing GZMB KO efficiency perform KO NK cells efficiency (n=5 healthy donors). All graphs are shown as mean±SEM.

FIGS. 34A-34H illustrate the recovery and function of IL15+IL18 pretreated NK cells after cryopreservation. FIG. 34A Recovery of NK cells pretreated with either a GZMB inhibitor or pan caspase inhibitor at 24 h after thawing (n=4 healthy donors). FIG. 34B Recovery of NK cells pretreated with IL-1 cytokine family after thawing (n=4 healthy donors). FIG. 34C NK cell recovery after pretreating NK cells with cytokines alone or in combination (n=10 healthy donors). FIG. 34D Time course study of NK cell recovery to identify the optimal duration of pretreatment with IL-15+IL-18 (n=3 healthy donors). FIG. 34E NK cell mediated K562 killing assay (n=4 healthy donors). FIG. 34F NK cell mediated ADCC killing assay with anti-CD20 antibody (n=3 healthy donors). FIG. 34G Heatmap of single cell secretome profile of IL-15+IL-18 cryopreserved, and non-cryopreserved NK cells treated with R848 for 24 h (n=3 healthy donors). FIG. 34H Caspase 3/7 activity luminescence assay at 9 hours after thawing of vehicle and IL-15+18 treated cells. Normality test was used to determine the distribution of the data, then two tailed t test was used for h, Two-way RM ANOVA was used for FIGS. 34E, 34F and 34H. All graphs are shown as mean±SEM.

FIGS. 35A-35F illustrate GZMB levels after thawing and PMA/Ionomycin treatment. FIG. 35A GZMB level in IL-15+IL-18 treated cells 24 hours after thawing (n=4 healthy donors). FIG. 35B GZMB secretion by IL-15+IL-18 treated cells 24 hours after thawing (n=4 healthy donors). FIG. 35C DMSO or PMA/ionomycin treated NK cell degranulation after 4 hours (n=4 healthy donors). FIG. 35D DMSO or PMA/ionomycin treated NK cell GZMB MFI after 4 hours (n=4 healthy donors). FIG. 35E DMSO or PMA/ionomycin treated NK cell recovery after thawing (n=4 healthy donors). FIG. 35F BCL2L1 KO efficiency on DNA and protein level (n=4 healthy donors). Normality test was used to determine the distribution of the data, then two tailed t test was used for FIGS. 35A, 35C, and 35D and non-parametric Wilcoxon matched pairs two-tailed t test was used for FIGS. 35B and 35E. All graphs are shown as mean±SEM.

FIGS. 36A-36C illustrate NK cells phenotype after IL-15+18 treatment and cryopreservation. FIGS. 36A and 36B illustrate the sample clean up and gating strategy. FIG. 36C illustrates NK cells percentage in each cluster at time 0 hours, 1, 3, 7, and 10 days after thawing (n=3 healthy donors). All graphs are shown as mean f SEM

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of 20% or ±10%, more preferably ±5%, even more preferably ±1%, and still more preferably ±0.1% from the specified value, as such variations are appropriate to perform the disclosed methods.

“Activation,” as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies (scFv) and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to. Fab, Fab′, F(ab′)2, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. Kappa and lambda light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” or “Ag” as used herein is defined as a molecule that provokes an immune response. This immune response may involve either antibody production, or the activation of specific immunologically competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequence or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, the term “autologous” is meant to refer to any material derived from the same individual to which it is later to be re-introduced into the individual.

“Allogeneic” refers to any material derived from a different animal of the same species.

“Xenogeneic” refers to any material derived from an animal of a different species.

The term “chimeric antigen receptor” or “CAR.” as used herein, refers to an artificial T cell receptor that is engineered to be expressed on an immune effector cell and specifically bind an antigen. CARs may be used as a therapy with adoptive cell transfer. T cells are removed from a patient and modified so that they express the receptors specific to a particular form of antigen. In some embodiments, the CARs have specificity to a selected target, for example a B cell surface receptor. CARs may also comprise an intracellular activation domain, a transmembrane domain and an extracellular domain comprising a tumor associated antigen binding region. In some aspects, CARs comprise an extracellular domain comprising an anti-B cell binding domain fused to CD3-zeta transmembrane and intracellular domain

The term “cleavage” refers to the breakage of covalent bonds, such as in the backbone of a nucleic acid molecule or the hydrolysis of peptide bonds. Cleavage can be initiated by a variety of methods, including, but not limited to, enzymatic or chemical hydrolysis of a phosphodiester bond. Both single-stranded cleavage and double-stranded cleavage are possible. Double-stranded cleavage can occur as a result of two distinct single-stranded cleavage events. DNA cleavage can result in the production of either blunt ends or staggered ends. In certain embodiments, fusion polypeptides may be used for targeting cleaved double-stranded DNA.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for the ability to bind antigens using the functional assays described herein.

“Co-stimulatory ligand,” as the term is used herein, includes a molecule on an antigen presenting cell (e.g., an aAPC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. A co-stimulatory ligand can include, but is not limited to, CD7, B7-1 (CD80), B7-2 (CD86), PD-L1, PD-L2, 4-1BBL, OX40L, inducible costimulatory ligand (ICOS-L), intercellular adhesion molecule (ICAM). CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as, but not limited to, CD27. CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include but are not limited to an MHC class I molecule, BTLA and a Toll ligand receptor.

A “co-stimulatory signal”, as used herein, refers to a signal, which in combination with a primary signal, such as TCR/CD3 ligation, leads to T cell proliferation and/or upregulation or downregulation of key molecules.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “downregulation” as used herein refers to the decrease or elimination of gene expression of one or more genes.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, anti-tumor activity as determined by any means suitable in the art.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “expand” as used herein refers to increasing in number, as in an increase in the number of T cells. In one embodiment, the T cells that are expanded ex vivo increase in number relative to the number originally present in the culture. In another embodiment, the T cells that are expanded ex vivo increase in number relative to other cell types in the culture. The term “ex vivo,” as used herein, refers to cells that have been removed from a living organism, (e.g., a human) and propagated outside the organism (e.g., in a culture dish, test tube, or bioreactor).

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies can comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature, 321: 522-525, 1986: Reichmann et al., Nature, 332: 323-329, 1988; Presta, Curr. Op. Struct. Biol., 2: 593-596, 1992.

“Fully human” refers to an immunoglobulin, such as an antibody, where the whole molecule is of human origin or consists of an amino acid sequence identical to a human form of the antibody.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

The term “immunoglobulin” or “Ig,” as used herein is defined as a class of proteins, which function as antibodies. Antibodies expressed by B cells are sometimes referred to as the BCR (B cell receptor) or antigen receptor. The five members included in this class of proteins are IgA, IgG. IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function but may serve as an antigen receptor. IgE is the immunoglobulin that mediates immediate hypersensitivity by causing release of mediators from mast cells and basophils upon exposure to allergen.

The term “immune response” as used herein is defined as a cellular response to an antigen that occurs when lymphocytes identify antigenic molecules as foreign and induce the formation of antibodies and/or activate lymphocytes to remove the antigen.

When “an immunologically effective amount,” “an autoimmune disease-inhibiting effective amount,” or “therapeutic amount” is indicated, the precise amount of the compositions of the present invention to be administered can be determined by a physician or researcher with consideration of individual differences in age, weight, tumor size, extent of infection or metastasis, and condition of the patient (subject).

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “knockdown” as used herein refers to a decrease in gene expression of one or more genes.

The term “knockout” as used herein refers to the ablation of gene expression of one or more genes.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

The term “limited toxicity” as used herein, refers to the peptides, polynucleotides, cells and/or antibodies of the invention manifesting a lack of substantially negative biological effects, anti-tumor effects, or substantially negative physiological symptoms toward a healthy cell, non-tumor cell, non-diseased cell, non-target cell or population of such cells either in vitro or in vivo.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating.” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “overexpressed” tumor antigen or “overexpression” of a tumor antigen is intended to indicate an abnormal level of expression of a tumor antigen in a cell from a disease area like a solid tumor within a specific tissue or organ of the patient relative to the level of expression in a normal cell from that tissue or organ. Patients having solid tumors or a hematological malignancy characterized by overexpression of the tumor antigen can be determined by standard assays known in the art.

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide.” “polypeptide.” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

A “Sendai virus” refers to a genus of the Paramyxoviridae family. Sendai viruses are negative, single stranded RNA viruses that do not integrate into the host genome or alter the genetic information of the host cell. Sendai viruses have an exceptionally broad host range and are not pathogenic to humans. Used as a recombinant viral vector, Sendai viruses are capable of transient but strong gene expression.

A “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the plasma membrane of a cell.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-beta, and/or reorganization of cytoskeletal structures, and the like.

A “stimulatory molecule.” as the term is used herein, means a molecule on a T cell that specifically binds with a cognate stimulatory ligand present on an antigen presenting cell.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

A “target site” or “target sequence” refers to a genomic nucleic acid sequence that defines a portion of a nucleic acid to which a binding molecule may specifically bind under conditions sufficient for binding to occur.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (α) and beta (β) chain, although in some cells the TCR consists of gamma and delta (γ/δ) chains. TCRs may exist in alpha/beta and gamma/delta forms, which are structurally similar but have distinct anatomical locations and functions. Each chain is composed of two extracellular domains, a variable and constant domain. In some embodiments, the TCR may be modified on any cell comprising a TCR, including, for example, a helper T cell, a cytotoxic T cell, a memory T cell, regulatory T cell, natural killer T cell, and gamma delta T cell.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The invention of the present disclosure is based on the unexpected finding that the pre-treatment of isolated immune cells or precursor cells thereof, such as natural killer (NK) cells and T cells, with IL-18 cytokine or a combination of IL-18 and IL-15 cytokines prior to cryopreservation can improve the viability and cytotoxic function of the cells after thawing. This finding has particular significance to the field of cellular immunotherapy, due to the fact that NK cells do not endure cryopreservation well, often displaying poor viability and inhibited function after thawing using current methods.

### Cryopreserved Immune Cells in Immunotherapy

Natural killer cells, or NK cells, are lymphocytes that descend from the common hematopoietic progenitor of B and T cells. However, unlike B and T cells, NK cells are considered to be part of the innate immune response in that they express receptors which have relatively little sequence variation, and respond quickly to antigens without the need for priming by antigen presenting cells or prior activation by other cytokine-secreting cells. NK cells were originally described for their cytotoxic ability, particularly against tumor cells and virally-infected cells, hence the name “natural killer”. Upon contacting a potential target cell, NK cell activation is controlled by a balance of signals received by activating and inhibitory receptors. One of the most powerful inhibitory signals is the presence of major histocompatibility complex (MHC, on mice)/human leukocyte antigen (HLA, on humans) class I complexes on the target cell surface. Because MHC/HLA class I complexes present self-antigens to T cells, many cancers and viruses down-regulate MHC/HLA class I expression, and loss of these proteins encourages NK cell activation. Once activated, NK cells secrete granules comprising cytotoxic molecules such as perform and granzymes, which lead to lysis of the target cells. As such, NK cells form a powerful innate defense system against viruses and cancerous cells. Activated NK cells also secrete other activation-associated cytokines and chemokines such as interferon-gamma (IFNγ) and tumor necrosis factor alpha (TNFα) which help to modulate adaptive immune responses from dendritic cells, B cells, and T cells.

In humans, NK cells are traditionally identified by the absence of CD3 and the presence of CD56 on their membrane surfaces. In mice, the lack of CD3 is accompanied by the presence of the NK1.1 or CD49b antigens, depending on the strain. In both humans and mice, the presence of the natural cytotoxic receptor NKp46 is also used to identify NK cells. Additional markers such as lack of c-kit expression can be used to distinguish human NK cells, while in mice, NK cells can be further characterized by the lack of CD49a and positive Eomes expression.

Given their significant inherent cytotoxicity and natural role in killing cancerous, virally infected, and otherwise abnormal cells, NK cells have been an attractive as a subject for use in cellular immunotherapies against a number of diseases, including cancer. The fact that NK cells do not require MHC/HLA matching, and still demonstrate target specificity even in allogeneic recipients only further highlights their clinical utility. Indeed, there is an increased interest in using NK cells as host cells for chimeric antigen receptors (CARs), where they offer the promise of being truly “off the shelf” therapeutics which could be used in allogeneic hosts without the need for HLA matching or carrying the risks of developing graft versus host disease (GVHD).

A significant obstacle to the widespread development and adoption of NK cell-based immunotherapies is the relative difficulty in expanding and cryopreserving large numbers of NK cells. For the successful clinical use of NK cells, large-scale production of highly purified NK cells is critical. Most NK cell expansion strategies achieve this goal by using feeder cells, such as irradiated peripheral blood mononuclear cells (PBMCs), Epstein-Barr virus-transformed lymphoblastoid cell lines, or gene-modified, irradiated K562 cells. After large-scale production of NK cells, cryopreservation of expanded NK cells until use is a necessary step due the fact that expanded NK cells have a short “shelf life”. Unfortunately, NK cells which are directly cryopreserved using common methods often display poor viability after thawing, even after adoptive transfer directly into recipients or culture in permissive media comprising optimal concentrations of cytokines and feeder target cells. Likewise, there are concerns regarding the activity of cryopreserved NK cells, as cryopreservation of expanded NK cells has been shown to reduce the expression of NKG2D and TRAIL and impair cytotoxicity.

As such, in some aspects, the invention of the current disclosure provides a method for cryopreserving isolated immune cells or precursor cells thereof (e.g. NK cells) comprising isolating or purifying the immune cell or precursor cell thereof from a specimen from a subject, culturing and expanding the immune cell or precursor cell thereof for at least 7 days, pre-treating the expanded immune cells or precursor cells thereof for at least 24 hours with IL-18 at a concentration between 1.0 and 500 ng/ml, resuspending the pre-treated immune cell or precursor cell thereof in a cryoprotective medium and freezing the cell at −80° C., and storing the cryopreserved cell in liquid nitrogen (N2). In some embodiments, the method further comprises pre-treating the expanded immune cells or precursors thereof with an additional cytokine. In certain embodiments, the additional cytokine is IL-15, and the IL-15 is used at a concentration between 1.0 and 500 ng/ml. In some embodiments, the pretreatment with IL-18 maintains viability and cytotoxic function of the cryopreserved immune cell or precursor cell thereof after thawing. In some embodiments, the immune cell or precursor thereof is a natural killer (NK) cell or NK cell precursor cell. In some embodiments, the immune cell is a T cell or T cell precursor cell.

### Pre-Treatment Cytokines

In some aspect, the invention of the present disclosure provides methods of cryopreserving immune cells (e.g. NK cells or T cells), as well as methods of producing compositions comprising cryopreserved immune cells (e.g. NK cells and T cells). In some embodiments, these methods involved the pre-treatment of in vitro or ex vivo expanded immune cells (e.g NK cells or T cells) with cytokines (e.g. IL-18 and IL-15) just prior to cryopreservation. As disclosed elsewhere herein, pre-treated immune cells (e.g. NK cells or T cells) have higher viability and greater cytotoxic function after thawing as compared to cryopreserved immune cells (e.g. NK cells or T cells) which have not undergone cytokine pre-treatment. In some embodiments, IL-18 is used alone as a pre-treatment, provided either as an exogenous, recombinant cytokine added as a supplement to the culture medium, or as a membrane-bound fusion protein expressed by feeder cells, which are then co-cultured with the immune cells (e.g. NK cells or T cells). In some embodiments, IL-18 is used in combination with IL-15, which is provided as an exogenous, recombinant cytokine added as a supplement to culture media, or administered to subjects having received an adoptive transfer of IL-18 pre-treated immune cells (e.g. NK cells or T cells).

IL-18, also known as interferon-gamma inducing factor, is a proinflammatory cytokine of the IL-1 family. Like other IL-1 cytokines, such as IL-1β, IL-18 is translated as an inactive precursor, which requires processing by caspase-1 into an active cytokine. The precursor form of IL-18 is produced constitutively in most normal tissues, and it's activity is balanced by the presence of a high affinity IL-18 binding protein. IL-18 plays a major role in stimulating the production of IFN-γ from NK and T cells. In certain embodiments

IL-15 is a cytokine that shares similar pro-inflammatory functions with IL-2, such as the ability to stimulate B and T cell immune responses. IL-15 is a pleiotropic cytokine, constitutively expressed by many types of cells including macrophages, monocytes, dendritic cells (DCs), T cell, epithelial cells, fibroblasts, keratinocytes, and nerve cells. IL-15 plays a key role in lymphocyte and NK cell function and maintenance. IL-15 has been demonstrated to stimulate the proliferation, activation, and expansion of NK cells.

### IL-18 Fusion Proteins

In some aspects, the invention of the present disclosure also provides a fusion protein comprising a first protein domain and a second protein domain separated by a linker, wherein the first protein domain comprises IL-18 peptide and the second protein domain comprises a human CD4 receptor. In some embodiments, the IL-18 peptide is a mature IL-18 peptide. In some embodiments, the fusion protein further comprises CD4 signal peptide. In some embodiments, the fusion protein is membrane-bound. As used herein, the terms “IL18-CD4 fusion protein”, “CD4-IL-18 fusion protein”, and “IL-18/CD4 fusion protein” are used interchangeably to refer to the fusion proteins of the present disclosure.

In some embodiments of the invention of the current disclosure, the pre-treatment of cultured or ex vivo expanded primary NK cells prior to cryopreservation improves the viability and cytotoxic function of the NK cells after thawing. The term “pre-treatment”, as used herein, means contacting the cultured or expanded NK or T cells with the IL-18 cytokine for a period of time prior to the freezing of the cells. In some embodiments, the pre-treatment occurs for between 12 and 48 hours prior to freezing. In some preferred embodiments, the pre-treatment occurs for 24 hours prior to freezing. In some embodiments, the IL-18 is provided to the NK cells in the form of a membrane-bound fusion protein wherein the IL-18 peptide is fused to a human CD4 receptor protein via a flexible linker domain. This fusion protein is then expressed on irradiated feeder cells co-cultured with the NK cells.

In some embodiments, the fusion protein comprising the IL-18 peptide is expressed on cells which generate membrane-derived vesicles or exosomes which comprise the fusion protein or on cells which can be used in the manufacture of membrane vesicles or exosomes which comprise the fusion protein. Membrane vesicles, including exosomes and the like, are nanometer-scale lipid vesicles that are produced by many cell types that can contain proteins, nucleic acids, and other molecules derived from the cells from which they are derived. Vesicles and exosomes can be used to transfer molecules between cells in vitro and in vivo. In some embodiments of the current invention, vesicles or exosomes which express the membrane-anchored IL-18 peptides or fusion peptides disclosed herein are used in the methods of the invention. In some embodiments, K562 cells are used as a source of these vesicles or exosomes. It also contemplated that any cell line which is known in the art to be amenable to the production of membrane vesicles or exosomes is appropriate for use in generating exosomes comprising the fusion protein comprising the IL-18 peptide.

In some embodiments, the fusion protein further comprises a CD4 leader sequence. In some embodiments, the fusion protein comprises certain amino acid changes in the IL-18 and/or CD4 domains which stabilize the fusion protein, but which differ from the wildtype human mature IL-18 and/or CD4 sequences. In some embodiments, the leader sequence is derived from human CD4.

In some embodiments, the IL-18 peptide is anchored to the cell or exosome surface through the use of another anchor or transmembrane domain. Other anchor domains that are known in the art, and could also be used to generate the IL-18 fusion peptides of the invention include, but are not limited to transmembrane domains derived from CD8a, CD8b, CD28, CD45, Ig CH2-CH3, among others. It is also contemplated that the IL-18 peptide could be anchored to the membrane via a lipid anchor, such as a glycosylphosphatidylinosotol (GPI) based anchor, an N-myristoyl based anchor, an S-palmitoyl based anchor, and the like. A skilled artisan would be able to select an appropriate membrane anchor for use with the IL-18 fusion peptides of the current disclosure.

In some embodiments, the fusion protein further comprises a selectable tag or marker domain. In some embodiments, the selectable tag or marker is a 6× histidine tag. It is also contemplated that the IL-18-CD4 fusion protein of the invention could comprise any selectable tag or affinity tag or selectable marker known in the art including, but not limited to a myc tag, a chitin binding protein domain (CBP), a maltose binding protein domain (MBP), a 6× histidine (6×his) tag, a glutathione-S-transferase (GST) tag, a V5-tag, a HA-tag, a Spot-tag, a T7-tag, and a NE-tag, among others. In some embodiments, the selectable tag or marker comprises a fluorescence domain such as GFP, RFP, YFP, CBG, and the like.

In some embodiments, the IL-18 is the functionally active mature form of IL-18. In some embodiments, the IL-18 domain comprises a peptide which comprises the amino acid sequence set forth in SEQ ID NO: 3. Tolerable variations of the IL-18 sequence will be known to those of skill in the art. For example, in some embodiments the IL-18 peptide comprises an amino acid sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence set forth in SEQ ID NO: 3.

In some embodiments, the CD4 receptor domain comprises a peptide which comprises the amino acid sequence set forth in SEQ ID NO: 6. Tolerable variations of the CD4 sequence will be known to those of skill in the art. For example, in some embodiments the CD4 peptide comprises an amino acid sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence set forth in SEQ ID NO: 6.

In some embodiments, the IL-18-CD4 fusion protein further comprises a signal peptide sequence on the amino or carboxy terminus of the protein. In certain embodiments, the signal peptide is derived from a human CD4 receptor. In certain embodiments, the CD4 signal peptide domain comprises the amino acid sequence set forth in SEQ IN NO: 2. Tolerable variations of the CD4 signal peptide sequence will be known to those of skill in the art. For example, in some embodiments the CD4 signal peptide comprises an amino acid sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence set forth in SEQ ID NO: 2.

In some embodiments, the IL-18-CD4 fusion protein comprises the amino acid sequence set forth in SEQ ID NO: 1 or SEQ ID NO: 13. Tolerable variations of the IL-18-CD4 fusion are also disclosed herein and will be recognized to those of skill in the art. For example, in some embodiments the IL-18-CD4 fusion protein comprises an amino acid sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence set forth in SEQ ID NO: 1 or SEQ ID NO: 13.

In some embodiments, the IL-18 and CD4 domains of the fusion proteins disclosed herein are separated by a flexible linker domain. A “linker” is herein also referred to as “linker sequence” “spacer” “tethering sequence” or grammatical equivalents thereof. A “linker” as referred herein connects two distinct molecules that by themselves possess ligand binding, catalytic activity, or are naturally expressed and assembled as separate polypeptides, or comprise separate domains of the same polypeptide. For example, a soluble signaling molecule and a membrane surface bound receptor. A number of strategies can be used to covalently link molecules together. Linkers described herein can be utilized to join a peptide domain comprising a cytokine, chemokine, or similar signaling molecule and a membrane bound receptor molecule; or can be used to tether a soluble cytokine, chemokine, or similar signaling molecule to the N- or C-terminus of a membrane-bound polypeptide. These include but are not limited to polypeptide linkages between N- and C-termini of proteins or protein domains, linkage via disulfide bonds, and linkage via chemical cross-linking reagents. In one aspect of this embodiment, the linker is a peptide bond, generated by recombinant techniques or peptide synthesis. The linker peptide can predominantly include the following amino acid residues: Gly, Ser, Ala, or Thr. The linker peptide should have a length that is adequate to link two molecules in such a way that they assume the correct conformation relative to one another so that they retain the desired activity. In one embodiment, the linker is from about 1 to 50 amino acids in length or about 1 to 30 amino acids in length. In one embodiment, linkers of 1 to 20 amino acids in length can be used. Useful linkers include glycine-serine polymers, including for example (GS)n, (GSGGS)n (SEQ ID NO: 14), (GGGGS)n (SEQ ID NO: 15), and (GGGS)n (SEQ ID NO: 16), where n is an integer of at least one, glycine-alanine polymers, alanine-serine polymers, and other flexible linkers. Alternatively, a variety of non-proteinaceous polymers, including but not limited to polyethylene glycol (PEG), polypropylene glycol, polyoxyalkylenes, or copolymers of polyethylene glycol and polypropylene glycol, can find use as linkers.

In some embodiments, the IL-18-CD4 fusion protein of the invention comprises a G4S linker. In some embodiments, the linker comprises the amino acid sequence set forth in SEQ ID NO: 5.

### Methods of Treatment

In some aspects, the invention of the present disclosure provides a method for treating a disease in a subject in need thereof, comprising administering to the subject an effective amount of a composition comprising the immune cells (e.g. NK cells or T cells) or precursor cells thereof produced by the methods disclosed herein. In some cases, the subject has cancer, an acute viral infection, or a chronic infection (e.g. viral infection). The composition can include a pharmaceutical composition and further include a pharmaceutically acceptable carrier.

Treatment refers to a method that seeks to improve or ameliorate the condition being treated. With respect to cancer, treatment includes, but is not limited to, reduction of tumor volume, reduction in growth of tumor volume, increase in progression-free survival, or overall life expectancy. In certain embodiments, treatment will affect remission of a cancer being treated. In certain embodiments, treatment encompasses use as a prophylactic or maintenance dose intended to prevent reoccurrence or progression of a previously treated cancer or tumor. It is understood by those of skill in the art that not all individuals will respond equally or at all to a treatment that is administered, nevertheless these individuals are considered to be treated.

In certain embodiments, the disease is cancer. In certain embodiments, the immune cells produced by the methods of the invention are administered to the subject in order to treat the cancer. In certain embodiments, the immune cell is an NK cell. In certain embodiments, the immune cell is a T lymphocyte.

In certain embodiments, the method further comprises administering one or more additional therapeutics or interventions. In certain embodiments, the additional therapeutic is a cytokine, chemokine, or the like. In certain embodiments, the cytokine is IL-15.

There is no limitation on such additional therapeutics or interventions, which can include any therapeutic agents or small molecule drugs that is helpful for treating the subject in need thereof. In some embodiments, the additional therapeutics or interventions are administered with the immune cells or compositions described herein as a combination therapy. Non-limiting examples of additional therapeutics or interventions include chemotherapy (e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine), radiation therapy, immunotherapy, and other targeted therapy.

In certain embodiments, the cancer or tumor is a solid cancer or tumor. In certain embodiments, the cancer or tumor is a blood cancer or tumor. In certain embodiments, the cancer or tumor comprises breast, heart, lung, small intestine, colon, spleen, kidney, bladder, head, neck, ovarian, prostate, brain, pancreatic, skin, bone, bone marrow, blood, thymus, uterine, testicular, and liver tumors. In certain embodiments, tumors which can be treated with the antibodies of the present disclosure comprise adenoma, adenocarcinoma, angiosarcoma, astrocytoma, epithelial carcinoma, germinoma, glioblastoma, glioma, hemangioendothelioma, hemangiosarcoma, hematoma, hepatoblastoma, leukemia, lymphoma, medulloblastoma, melanoma, neuroblastoma, osteosarcoma, retinoblastoma, rhabdomyosarcoma, sarcoma and/or teratoma. In certain embodiments, the tumor/cancer is selected from the group of acral lentiginous melanoma, actinic keratosis, adenocarcinoma, adenoid cystic carcinoma, adenomas, adenosarcoma, adenosquamous carcinoma, astrocytic tumors. Bartholin gland carcinoma, basal cell carcinoma, bronchial gland carcinoma, capillary carcinoid, carcinoma, carcinosarcoma, cholangiocarcinoma, chondrosarcoma, cystadenoma, endodermal sinus tumor, endometrial hyperplasia, endometrial stromal sarcoma, endometrioid adenocarcinoma, ependymal sarcoma, Swing's sarcoma, focal nodular hyperplasia, gastronoma, germ line tumors, glioblastoma, glucagonoma, hemangioblastoma, hemangioendothelioma, hemangioma, hepatic adenoma, hepatic adenomatosis, hepatocellular carcinoma, insulinite, intraepithelial neoplasia, intraepithelial squamous cell neoplasia, invasive squamous cell carcinoma, large cell carcinoma, liposarcoma, lung carcinoma, lymphoblastic leukemia, lymphocytic leukemia, leiomyosarcoma, melanoma, malignant melanoma, malignant mesothelial tumor, nerve sheath tumor, medulloblastoma, medulloepithelioma, mesothelioma, mucoepidermoid carcinoma, myeloid leukemia, neuroblastoma, neuroepithelial adenocarcinoma, nodular melanoma, osteosarcoma, ovarian carcinoma, papillary serous adenocarcinoma, pituitary tumors, plasmacytoma, pseudosarcoma, prostate carcinoma, pulmonary blastoma, renal cell carcinoma, retinoblastoma, rhabdomyosarcoma, sarcoma, serous carcinoma, squamous cell carcinoma, small cell carcinoma, soft tissue carcinoma, somatostatin secreting tumor, squamous carcinoma, squamous cell carcinoma, undifferentiated carcinoma, uveal melanoma, verrucous carcinoma, vagina/vulva carcinoma, VIPpoma, and Wilm's tumor. In certain embodiments, the tumor/cancer to be treated with one or more antibodies of the present disclosure comprise brain cancer, head and neck cancer, colorectal carcinoma, acute myeloid leukemia, pre-B-cell acute lymphoblastic leukemia, bladder cancer, astrocytoma, preferably grade II, III or IV astrocytoma, glioblastoma, glioblastoma multiforme, small cell cancer, and non-small cell cancer, preferably non-small cell lung cancer, lung adenocarcinoma, metastatic melanoma, androgen-independent metastatic prostate cancer, androgen-dependent metastatic prostate cancer, prostate adenocarcinoma, and breast cancer, preferably breast ductal cancer, and/or breast carcinoma. In certain embodiments, the cancer treated with the antibodies of this disclosure comprises glioblastoma. In certain embodiments, the cancer treated with one or more antibodies of this disclosure comprises pancreatic cancer. In certain embodiments, the cancer treated with one or more antibodies of this disclosure comprises ovarian cancer. In certain embodiments, the cancer treated with one or more antibodies of this disclosure comprises lung cancer. In certain embodiments, the cancer treated with one or more antibodies of this disclosure comprises prostate cancer. In certain embodiments, the cancer treated with one or more antibodies of this disclosure comprises colon cancer. In certain embodiments, the cancer treated comprises glioblastoma, pancreatic cancer, ovarian cancer, colon cancer, prostate cancer, or lung cancer. In a certain embodiment, the cancer is refractory to other treatment. In a certain embodiment, the cancer treated is relapsed.

In some embodiments, the cancer comprises a melanoma (e.g., metastatic malignant melanoma), a prostate cancer (for example hormone refractory prostate adenocarcinoma), a head and neck cancer (for example, squamous cell carcinoma of the head and neck), a cervical cancer, a thyroid cancer, a glioblastoma, a glioma, leukemia, a lymphoma (for example, a B cell lymphoma), an adrenal gland cancer, an AIDS-associated cancer, an alveolar soft part sarcoma, an astrocytic tumor, bone cancer, a brain and spinal cord cancer, a metastatic brain tumor, a carotid body tumor, a chondrosarcoma, a chordoma, a chromophobe renal cell carcinoma, a clear cell carcinoma, cutaneous benign fibrous histiocytoma, a desmoplastic small round cell tumor, an ependymoma, a Ewing's tumor, an extra skeletal myxoid chondrosarcoma, a fibrogenesis imperfecta ossium, a fibrous dysplasia of the bone, a gallbladder or bile duct cancer, a gestational trophoblastic disease, a germ cell tumor, a hematological malignancy, hepatocellular carcinoma, an islet cell tumor, a Kaposi's sarcoma, a kidney cancer, a lipoma/benign lipomatous tumor, a liposarcoma/malignant lipomatous tumor, a medulloblastoma, a meningioma, a Merkel cell carcinoma, a multiple endocrine neoplasia, a multiple myeloma, a myelodysplasia syndrome, a neuroblastoma, a neuroendocrine tumor, a papillary thyroid carcinoma, a parathyroid tumor, a pediatric cancer, a peripheral nerve sheath tumor, a phaeochromocytoma, a pituitary tumor, a prostate cancer, a posterior uveal melanoma, a rare hematologic disorder, a renal metastatic cancer, a rhabdoid tumor, a rhabdomyosarcoma, a sarcoma, a soft-tissue sarcoma, a squamous cell cancer, a stomach cancer, a synovial sarcoma, a testicular cancer, a thymic carcinoma, a thymoma, a thyroid metastatic cancer, a uterine cancer, or any combination thereof.

In some cases, the cancer comprises cervical cancer, lung cancers, liver cancers, ovarian cancers, cancers of the skin including melanoma and squamous cell carcinoma, colon cancer, bladder cancer, breast cancer, kidney cancer, esophageal cancer, stomach cancer, pancreatic cancers, head cancer, and neck cancer.

In terms of the present disclosure, prophylactic, palliative, symptomatic and/or curative treatments may represent separate aspects of the disclosure. An immune cell or precursor thereof produced by the methods of the present disclosure can be administered parenterally, such as intravenously, such as intramuscularly, such as subcutaneously. An immune cell or precursor thereof produced by the methods of the present disclosure can be administered therapeutically (on demand).

### Introduction of Nucleic Acids

The present invention provides methods for producing/generating an immune cell or precursor cell thereof (e.g., a T cell or NK cell). In some embodiments, NK cells isolated or purified from subjects (e.g. in peripheral blood, cord blood, and the like), are expanded ex vivo prior to cryopreservation until further use. In some embodiments, prior to cryopreservation, the immune cells are pretreated with IL-18 or a mixture of IL-18 and IL-15. In some embodiments, the IL-18 is provided as a membrane bound fusion protein that is expressed by engineered feeder cells. In certain embodiments, the feeder cells are primary lymphocytes. In certain embodiments, the feeder cells are an immortalized cell line. In both cases the feeder cells are transfected or transformed with vectors encoding the IL-18 fusion protein (e.g. IL-18-CD4 fusion protein).

Methods of introducing nucleic acids into a cell include physical, biological and chemical methods. Physical methods for introducing a polynucleotide, such as RNA, into a host cell include calcium phosphate precipitation, lipofection, particle bombardment, microinjection, electroporation, and the like. RNA can be introduced into target cells using commercially available methods which include electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany). RNA can also be introduced into cells using cationic liposome mediated transfection using lipofection, using polymer encapsulation, using peptide mediated transfection, or using biolistic particle delivery systems such as “gene guns” (see, for example, Nishikawa, et al. Hum Gene Ther., 12(8):861-70 (2001).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

In some embodiments, a nucleic acid encoding a IL-18-CD4 fusion protein of the invention is introduced into a cell by an expression vector. Expression vectors comprising a nucleic acid encoding a subject IL-18-CD4 fusion protein are provided herein. Suitable expression vectors include lentivirus vectors, gamma retrovirus vectors, foamy virus vectors, adeno associated virus (AAV) vectors, adenovirus vectors, engineered hybrid viruses, naked DNA, including but not limited to transposon mediated vectors, such as Sleeping Beauty, Piggyback, and Integrases such as Phi31. Some other suitable expression vectors include herpes simplex virus (HSV) and retrovirus expression vectors.

Adenovirus expression vectors are based on adenoviruses, which have a low capacity for integration into genomic DNA but a high efficiency for transfecting host cells. Adenovirus expression vectors contain adenovirus sequences sufficient to: (a) support packaging of the expression vector and (b) to ultimately express the subject CAR in the host cell. In some embodiments, the adenovirus genome is a 36 kb, linear, double stranded DNA, where a foreign DNA sequence (e.g., a nucleic acid encoding a subject CAR) may be inserted to substitute large pieces of adenoviral DNA in order to make the expression vector of the present invention (see, e.g., Danthinne and Imperiale, Gene Therapy (2000) 7(20): 1707-1714).

Another expression vector is based on an adeno associated virus, which takes advantage of the adenovirus coupled systems. This AAV expression vector has a high frequency of integration into the host genome. It can infect non-dividing cells, thus making it useful for delivery of genes into mammalian cells, for example, in tissue cultures or in vivo. The AAV vector has a broad host range for infectivity. Details concerning the generation and use of AAV vectors are described in U.S. Pat. Nos. 5,139,941 and 4,797.368.

Retrovirus expression vectors are capable of integrating into the host genome, delivering a large amount of foreign genetic material, infecting a broad spectrum of species and cell types and being packaged in special cell lines. The retrovirus vector is constructed by inserting a nucleic acid (e.g., a nucleic acid encoding a subject CAR) into the viral genome at certain locations to produce a virus that is replication defective. Though the retrovirus vectors are able to infect a broad variety of cell types, integration and stable expression of the subject CAR, requires the division of host cells.

Lentivirus vectors are derived from lentiviruses, which are complex retroviruses that, in addition to the common retroviral genes gag, pol, and env, contain other genes with regulatory or structural function (see. e.g., U.S. Pat. Nos. 6,013,516 and 5,994,136). Some examples of lentiviruses include the human immunodeficiency viruses (HIV-1, HIV-2) and the simian immunodeficiency virus (SIV). Lentivirus vectors have been generated by multiply attenuating the HIV virulence genes, for example, the genes env, vif, vpr, vpu and nef are deleted making the vector biologically safe. Lentivirus vectors are capable of infecting non-dividing cells and can be used for both in vivo and ex vivo gene transfer and expression, e.g., of a nucleic acid encoding a subject CAR (see, e.g., U.S. Pat. No. 5,994,136).

Expression vectors including a nucleic acid of the present disclosure can be introduced into a host cell by any means known to persons skilled in the art. The expression vectors may include viral sequences for transfection, if desired. Alternatively, the expression vectors may be introduced by fusion, electroporation, biolistics, transfection, lipofection, or the like. The host cell may be grown and expanded in culture before introduction of the expression vectors, followed by the appropriate treatment for introduction and integration of the vectors. The host cells are then expanded and may be screened by virtue of a marker present in the vectors. Various markers that may be used are known in the art, and may include hprt, neomycin resistance, thymidine kinase, hygromycin resistance, etc. As used herein, the terms “cell,” “cell line,” and “cell culture” may be used interchangeably. In some embodiments, the host cell is an primary lymphocyte or a mixture thereof. In embodiments, the host cells in immortalized cell (e.g. a cell line).

Modified cells (e.g., comprising a subject IL-18-CD4 fusion protein) may be produced by stably transfecting host cells with an expression vector including a nucleic acid of the present disclosure. Additional methods to generate a modified cell of the present disclosure include, without limitation, chemical transformation methods (e.g., using calcium phosphate, dendrimers, liposomes and/or cationic polymers), non-chemical transformation methods (e.g., electroporation, optical transformation, gene electro transfer and/or hydrodynamic delivery) and/or particle-based methods (e.g., impalefection, using a gene gun and/or magnetofection).

Physical methods for introducing an expression vector into host cells include calcium phosphate precipitation, lipofection, particle bombardment, microinjection, electroporation, and the like. Methods for producing cells including vectors and/or exogenous nucleic acids are well-known in the art. See, e.g., Sambrook et al. (2001), Molecular Cloning: A Laboratory Manual, Cold Spring Harbor Laboratory, New York.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the nucleic acids in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

Moreover, the nucleic acids may be introduced by any means, such as transducing the expanded T cells, transfecting the expanded T cells, and electroporating the expanded T cells. One nucleic acid may be introduced by one method and another nucleic acid may be introduced into the T cell by a different method.

### Sources of Immune Cells

Prior to expansion, a source of NK cells or T cells is obtained from a subject. Non-limiting examples of subjects include humans, dogs, cats, mice, rats, and transgenic species thereof. Preferably, the subject is a human. NK cells or T cells can be obtained from a number of sources, including peripheral blood mononuclear cells, bone marrow, lymph node tissue, spleen tissue, umbilical cord, and tumors. In certain embodiments, any number of T cell lines or NK cell lines available in the art, may be used. NK cells or T cells can also be obtained from or differentiated from CD34+ cord blood stem cells and induced pluripotent stem cells (iPSCs). NK cells or T cells can also be obtained from the in vivo culture of NK cell lines or T cell lines. In certain embodiments, NK cells or T cells can be obtained from a unit of blood collected from a subject (e.g. a patient or normal donor) using any number of techniques known to the skilled artisan, such as Ficoll separation. In one embodiment, cells from the circulating blood of an individual are obtained by apheresis or leukapheresis. The apheresis product typically contains lymphocytes, including NK cells or T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. After washing, the cells may be resuspended in a variety of biocompatible buffers, such as, for example. Ca-free, Mg-free PBS. Alternatively, the undesirable components of the apheresis sample may be removed and the cells directly resuspended in culture media.

In another embodiment, NK cells or T cells are isolated from peripheral blood by lysing the red blood cells and depleting the monocytes, for example, by centrifugation through a PERCOLL™ gradient. Alternatively. NK cells or T cells can be isolated from umbilical cord. In any event, a specific subpopulation of NK cells or T cells can be further isolated by positive or negative selection techniques.

Enrichment of a T cell or NK cell population by negative selection can be accomplished using a combination of antibodies directed to surface markers unique to immune cells that are not the negatively selected cells. A preferred method is cell sorting and/or selection via negative magnetic immunoadherence or flow cytometry that uses a cocktail of monoclonal antibodies directed to cell surface markers present on the cells negatively selected.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion.

NK cells or T cells can also be frozen after the washing step, which does not require the monocyte-removal step. While not wishing to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, in a non-limiting example, one method involves using PBS containing 20% DMSO and 8% human serum albumin. or other suitable cell freezing media. The cells are then frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen. In certain embodiments, the NK cells or T cells are pre-treated by culturing in the presence of IL-18 or a combination of IL-18 and IL-15 for at least 24 hours prior to freezing. In certain embodiments, the IL-18 is provided via a feeder cell which expresses a membrane-bound fusion protein comprising IL-18 polypeptide fused to a human CD4 receptor via a flexible linker domain.

In one embodiment, the population of NK cells or T cells is comprised within cells such as peripheral blood mononuclear cells, cord blood cells, a purified population of NK cells or T cells, and a NK cell line or a T cell line. In another embodiment, peripheral blood mononuclear cells comprise the population of NK cells or T cells. In yet another embodiment, purified NK cells or T cells comprise the population of NK cells or T cells.

### Expansion of Immune Cells

As demonstrated by the data disclosed herein, expanding the NK cells or T cells by the methods disclosed herein can be multiplied by about 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater, and any and all whole or partial integers therebetween. In one embodiment, the NK cells or T cells expand in the range of about 20 fold to about 50 fold.

Following culturing, the NK cells or T cells can be incubated in cell medium in a culture apparatus for a period of time or until the cells reach confluency or high cell density for optimal passage before passing the cells to another culture apparatus. The culturing apparatus can be of any culture apparatus commonly used for culturing cells in vitro. Preferably, the level of confluence is 70% or greater before passing the cells to another culture apparatus. More preferably, the level of confluence is 90% or greater. A period of time can be any time suitable for the culture of cells in vitro. The NK cell or T cell medium may be replaced during the culture of the NK cells or T cells at any time. Preferably, the NK cell or T cell medium is replaced about every 2 to 3 days. The NK cells or T cells are then harvested from the culture apparatus whereupon the NK cells or T cells can be used immediately or pre-treated using the methods disclosed elsewhere herein followed by cryopreservation to be stored for use at a later time. In one embodiment, the invention includes pre-treating and cryopreserving the expanded NK cells or T cells. The cryopreserved NK cells or T cells are thawed prior to further clinical use.

In another embodiment, the method comprises isolating NK cells or T cells and expanding the NK cells or T cells. In another embodiment, the invention further comprises cryopreserving the NK cells or T cells prior to expansion. In yet another embodiment, the NK cells or T cells are first pre-treated with IL-18 or the combination of IL-18 and IL-15 for at least 8, 9, 10, 11, 12, 13, 14, 15, 16, 17, 18, 19, 20, 21, 22, 23, 24, or more hours prior to cryopreservation.

The culturing step as described herein can be very short, for example less than 24 hours such as 1, 2, 3, 4, 5, 6, 7, 8, 9, 10, 11, 12, 13, 14, 15, 16, 17, 18, 19, 20, 21, 22, or 23 hours. The culturing step as described further herein can be longer, for example 1, 2, 3, 4, 5, 6, 7, 8, 9, 10, 11, 12, 13, 14, or more days.

Various terms are used to describe cells in culture. Cell culture refers generally to cells taken from a living organism and grown under controlled condition. A primary cell culture is a culture of cells, tissues or organs taken directly from an organism and before the first subculture. Cells are expanded in culture when they are placed in a growth medium under conditions that facilitate cell growth and/or division, resulting in a larger population of the cells. When cells are expanded in culture, the rate of cell proliferation is typically measured by the amount of time required for the cells to double in number, otherwise known as the doubling time.

Each round of subculturing is referred to as a passage. When cells are sub cultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but is not limited to the seeding density, substrate, medium, and time between passaging.

In one embodiment, the cells may be cultured for several hours (about 3 hours) to about 14 days or any hourly integer value in between. Conditions appropriate for NK cells or T cell culture include an appropriate media (e.g., Minimal Essential Media or RPMI Media 1640 or, X-vivo 15, (Lonza)), that may contain factors necessary for proliferation and viability, including serum (e.g., fetal bovine or human serum), cytokines, or any other additives for the growth of cells known to the skilled artisan. For NK cells, cytokines include IL-2 and IL-15. For T cells, cytokines include IL-2, IFN-gamma, IL-4, IL-7, GM-CSF, IL-10, IL-12, IL-15, TGF-beta, and TNF-α, or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of NK cells or T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

Expansion of NK cells also requires the use of feeders such as tumor cell lines or PBMCs. Commonly used feeder cell lines are RPM18866, Epstein-Barr lymphoblastoid cell line (EBV-LCL), and K562. Stimulation with NK-sensitive K562 cells is known to augment NK cell proliferation to IL-2. IL-15, and IL-21 in combination. In certain embodiments, pre-treatment of NK cells and T cells includes the co-culture of expanded cells with feeder cells which have been modified or engineered to express an IL-18-CD4 fusion protein with or without the co-administration of additional IL-15 cytokine.

The medium used to culture the NK cells or T cells may include an agent that can co-stimulate the NK cells or T cells. For example, an agent that can stimulate T cells is an antibody to CD3, and an antibody to CD28. For example, an agent that can expand NK cells is a feeder cell. This is because, a cell isolated by the methods disclosed herein can be expanded approximately 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater. In one embodiment, the NK cell or T cells expand in the range of about 20 fold to about 50 fold, or more by culturing the electroporated population.

### Pharmaceutical Compositions

Pharmaceutical compositions of the present invention may comprise the modified T cell as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

The cells of the invention to be administered may be autologous, allogeneic or xenogeneic with respect to the subject undergoing therapy.

Cells of the invention can be administered in dosages and routes and at times to be determined in appropriate pre-clinical and clinical experimentation and trials. Cell compositions may be administered multiple times at dosages within these ranges. Administration of the cells of the invention may be combined with other methods useful to treat the desired disease or condition as determined by those of skill in the art.

It can generally be stated that a pharmaceutical composition comprising the modified NK cells described herein may be administered at a dosage of 10⁴ to 10⁹ cells/kg body weight, in some instances 10⁵ to 10⁶ cells/kg body weight, including all integer values within those ranges. NK cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

The administration of the NK cells of the invention may be carried out in any convenient manner known to those of skill in the art. The cells of the present invention may be administered to a subject by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In other instances, the cells of the invention are injected directly into a site of inflammation in the subject, a local disease site in the subject, a lymph node, an organ, a tumor, and the like.

It should be understood that the method and compositions that would be useful in the present invention are not limited to the particular formulations set forth in the examples. The following examples are put forth so as to provide those of ordinary skill in the art with a complete disclosure and description of how to make and use the cells, expansion and culture methods, and therapeutic methods of the invention, and are not intended to limit the scope of what the inventors regard as their invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the invention, and, as such, may be considered in making and practicing the invention. Particularly useful techniques for particular embodiments will be discussed in the sections that follow.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

The materials and methods employed in these experiments are now described.

Cells: All cells used herein were purified from consented healthy human donors. NK and NK cells were isolated from peripheral blood using either one of the following kits: (STEMCELL™ Technologies Catalog #15065 and Catalog #15061). mIL-21-producing K562 cells were used as feeder cells for NK cell expansion. The cells were irradiated at 1,000 Gy and then mixed with NK cells at a 1:10 (Effector:Target cells) ratio. NK cells were expanded using CD3-CD28 beads.

K562 (CRL-3343), Raji (CLL-86), and Jeko-1 (CRL-3006) cell lines were obtained from the ATCC and engineered to express click beetle green luciferase (CBG). K562 feeder cells expressing 41BBL, CD40, and membrane-bound IL-21 (K-IL21) were generated as previously described. The identity of all cell lines was verified by STR profiling (ATCC), and they were confirmed negative for mycoplasma (Cambrex MycoAlert®) following culture without antibiotics. Cells were cultured in RPMI 1640 (Thermo fisher #11875085) supplemented with 10% fetal bovine serum (FBS) (Seradigm #97068-086), and penicillin-streptomycin (Gibco #15140122).

NK cell isolation and expansion: Peripheral blood mononuclear cells (PBMCs) were obtained after written informed consent under University of Pennsylvania Institutional review board (IRB) approved protocol (#705906) from healthy adult donors NK cells were isolated and expanded for 15-20 days in G-Rex 6-well plates (Wilson Wolf #80240M) via seeding with irradiated K-IL21 feeder cells at an E:T ratio of 1:10 in X-VIVO 10 with Gent and Phenol Red (Lonza #BE04-380Q) supplemented with 10% human AB serum (Valley Biomedical #HP1022) and 100 U/ml of recombinant human IL-2 (Peprotech #200-02) (NK media). Fresh media was added to NK cells every 3 days as previously described.

Cytokines and inhibitors: IL-15 at (Peprotech #200-15), IL-12 (Peprotech #200-12), IL-18 (Invivogen #rcyec-hi118), IL-1a (Peprotech #200-200-01A), IL-1b ((Peprotech #200-01B). IL-33 (Peprotech #200-33), IL-36 (Peprotech #200-36), Pan Caspase Inhibitor Z VAD FMK (R&D Systems #FMK001), Brefeldin A Solution (1000×) (BioLegend #420601). Granzyme B Inhibitor II, Calbiochem (EMD Millipore #368055-1MG), Cell Activation Cocktail (BioLegend #423302) were used.

Flow cytometry and CyTOF: For flow cytometry antibodies, a ratio of 1:100 of antibody per 200,000 cells was used in 100 ul staining buffer. Cells were incubated at room temperature for 20 minutes then washed with staining buffer at 450gx for 5 minutes. Cells were then run on a LSRFortessa™ flow cytometer. For CyTOF antibodies, the manufacturer's recommended dilution 1:100 and instructions were used.

Antibody and viability Staining: The following kits and reagents were used: Absolute Counting Beads (Thermo Scientific C36950) CD56 (BioLegend #362552) CD3 (BioLegend #300420) Calcein-AM (BioLegend #425201) and Propidium Iodide (Thermo Fisher #P3566). Cells were stained according to the manufacture recommended protocols. Cells were assayed by flow cytometry.

Cytokines for cell activation: Recombinant human IL-18 (invivogen #rcyec-hi118) recombinant human IL-12 (PeproTech #200-12) and recombinant human IL-15 (PeproTech #200-15) were added to NK cell cultures for 24 hours. Cytokines were used between 50-500 ng/ml. The ApoTox-Glo™ Triplex Assay (Promega #G6320) was used a according to the manufacture recommended protocol.

NK cell cytotoxicity assay: NK cells were co-Cultured with luciferase-expressing K562 cells at 1:1, 1:2, and 1:4 ratios for 24 hours at 37° C. Luciferase substrate was added for 15 minutes at room temperature (~20-22° C.). Luminescence was read using a plate reader.

NK cell cryopreservation and thawing: NK cells were resuspended in NK media at 5-10 6 cells per ml. IL-15 and IL-18 were added at 50 and 250 ng/ml, respectively, while all other cytokines tested were added at 100 ng/ml 24 hours before cryopreservation unless otherwise stated. NK cells were washed with media then resuspended in 1 ml of CryoStor CS5 or CryoStor CS10 (STEMCELL #079331,07930) before freezing to −80° C. in a CoolCell® cell freezer (VWR #75779-712) overnight followed by transfer to the vapor phase of liquid nitrogen for long-term storage. NK cells were thawed in a 37° C. water bath. When only a small piece of ice remained, cells were added dropwise to NK cell media at ratio of 1:10 volume of cells to media. Cells were centrifuged for 3 minutes at 400×g. Media was removed and cells were resuspended in NK cells media at 2.5-10×10 6 cells per ml.

Degranulation assay: NK cells were co-cultured with K562 cells for 3 hours at 37° C. and then stained for flow cytometry analysis using a Fortessa flow cytometer. Percent degranulation was determined using the following gate CD3-CD56+CD107a+ (% CD3-CD56+CD107a+ cells/Total CD3-CD56+ cells)*100. Single cell cytokine release assay NK cells were thawed and rested in NK media for 24 hours prior to stimulation with 1 μg/ml R848 (Invivogen #tlrl-r848) for 16 hours. NK cells were then stained with cell trace violet membrane dye and loaded into IsoPlexis single cell human NK cell secretome chips, which were loaded into the IsoSpark Duo instrument and analyzed with IsoSpeak software according to the manufacturer's instructions.

Microscopy: Non-cryopreserved or cryopreserved cells were fixed and stained with FITC-conjugated anti-CD63 and AF647-conjugated anti-Granzyme B antibodies. Cells were then washed twice and loaded onto EZ Double Cytofunnel (Fisher Scientific #A78710005) with glass slides. Cytofunnel was spun down to attach cells to the glass slides at 600 RPM for 10 min using mounting media ProLong Gold Antifade Reagent DAPI (ThermoFisher #P36931). Incucyte imaging for caspase 3/7, Cytox NIR and killing assays NK cells were thawed and immediately stained with Incucyte Caspase-3/7 Dye for Apoptosis-RED (Sartorius #4704), and Incucyte Cytotox Dye for Counting Dead Cells, NIR (Sartorius #4846) according to the manufacturer's instructions. NK cells were co-cultured with GFP-expressing K562 or B2M KO Raji cells at 2:1, target to effector (T:E) ratio. Cells were incubated at 37° C. for 48 hours and target cells fluorescence was measured every 1.5 hours. ADCC assays using Jeko-1 cells as target cells were performed similarly to cytotoxicity assay but with addition of anti-CD20 (Bio X Cell #SIM0008) at 1 μg/ml.

Gene editing of NK cells and Raji cells: To generate B2M KO Raji cell line guide RNA targeting B2M was purchased form Synthego and used with SpyFi Cas9 Nucleases (Aldevron #9214-5MG). gRNA was mixed with Cas9 for 20 minutes at RT to form RNP complex and then electroporated into Raji cells using a LONZA 4D system with SF buffer (LONZA #V4LC-2520). Guide RNAs targeting Granzyme B. Perform and BCL2L1 were purchased form Synthego for RNP complexes were formed for 15 mins at room temperature and then NK cells were electroporated using the BTX system at 500 μv for 700 μs then added to complete media at 2.5×10⁶ cells/ml. Knockout efficiency was measured 3-4 days after electroporation by flow cytometry. To confirm KO on the DNA level these primers targeting Granzyme B, Perforin and BCL2L1 were used.

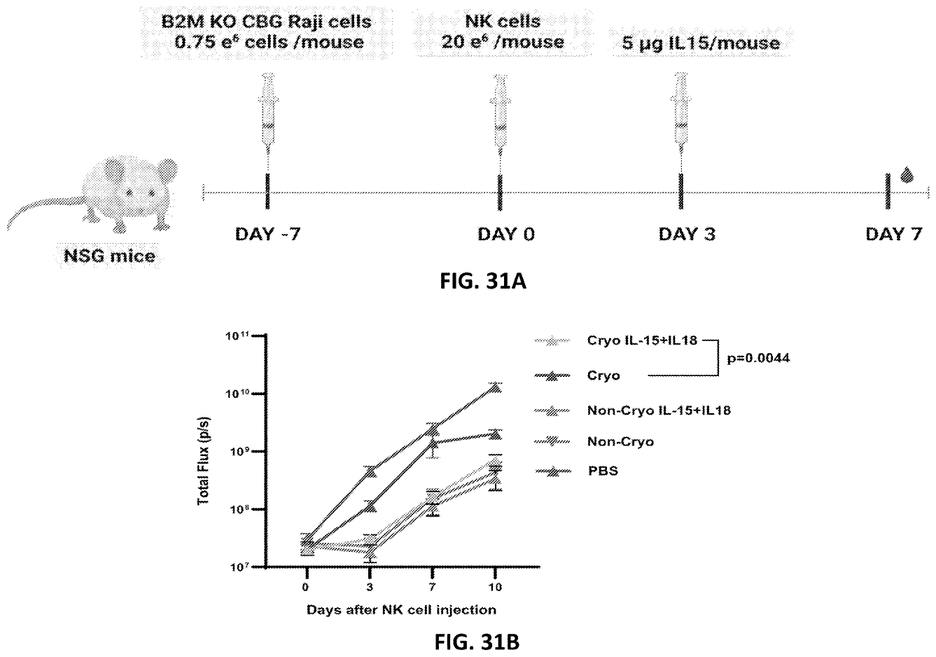

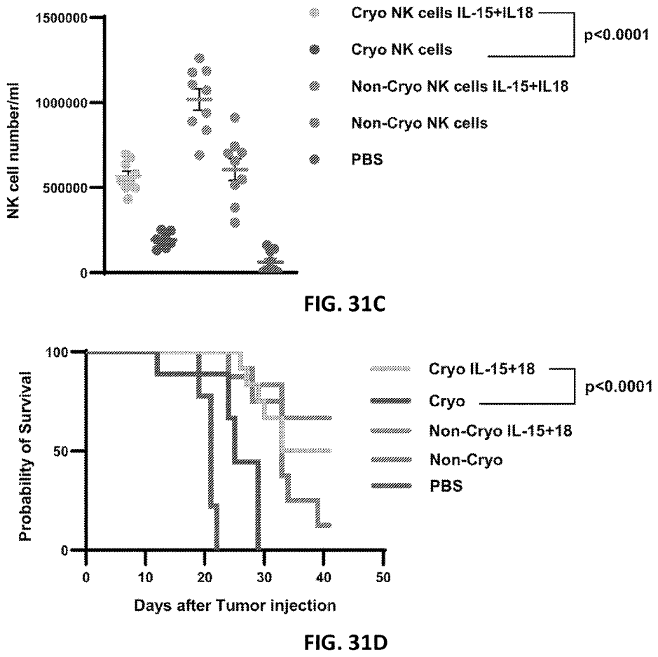

RNA sequencing and Nanostring Counter assay: Total RNA was isolated from NK cells using the RNeasy Mini Kit (QIAGEN #74104). Bulk RNA sequencing was performed at Novogen and Nanostring analysis was performed at the Wistar Institute genomic core. Bulk sequencing reads were aligned to reference genome using the Hisat2 v2.0.5. Feature Counts v1.5.0-p3 was used to count the uniquely mapped gene. For differential expression analysis the DESeq2R package (1.20.0) was used with Padj of 0.05 as described here. For Nanostring, data were analyzed using the LBL-10025-02_PanCancer-Pathways panel and online software (www.rosalind.bio). Disseminated Raji Lymphoma Model 6-8-week-old male NOD/scid/IL2rγ−/− NSG mice originally obtained from Jackson Laboratories were bred and maintained. Mice were injected with 750,000 B2M KO Raji cell line expressing CBG. Day 7 after tumor injection mice were randomized into groups (9 mice/group) then injected with NK cells as described in FIG. 31A. Mice were injected with 5 μg of IL-15 (peprotech #200-15) on day 3 NK after injection. Mice were imaged every 3 days starting at day 0 after NK cell injection. Cheek bleed was performed at day 7 after NK cell injection and NK cells were counted using Trucount tubes (BD #91-0786) according to the manufacture's protocol. Mice were euthanized if any of these conditioned were met; loss of more than 20% of body weight, Bioluminescent Imaging (BLI) of the tumor reach 1×10¹² or paralysis in one or both limbs. Mice were mentored 2-3 times a week.

Statistical Analyses: All statistical analyses were performed in GraphPad Prism (v9.0.1). The nature of the statistical test used is described in the figure legends, and corrections for multiple comparisons made where appropriate. A result ≤0.05 was considered statistically significant.

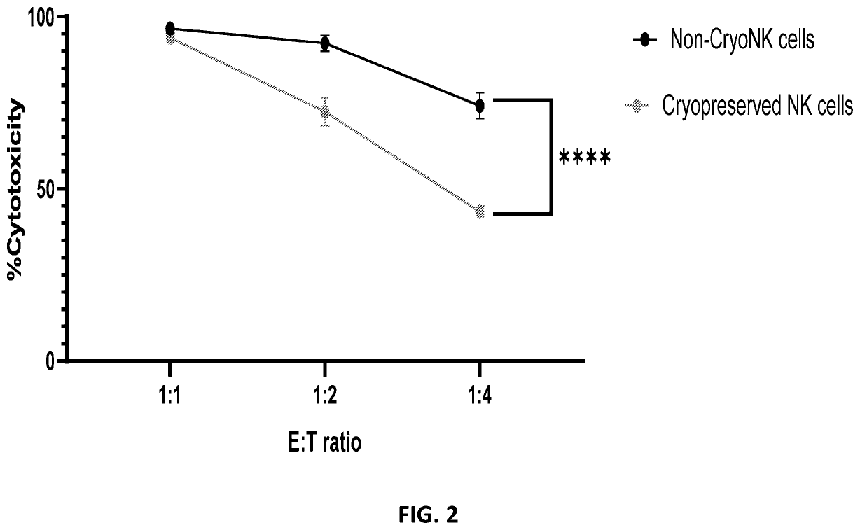

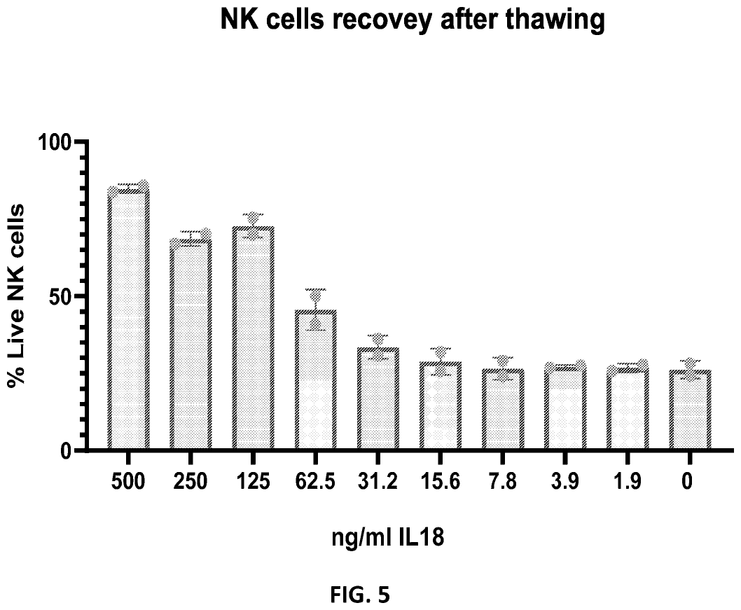

Software and Illustrations: For Flow Cytometry the BD FACSDiva™ v8.0.1 Software was used. Incucyte® Base Analysis Software 2022B Rev3, Leica SP8 laser scanning confocal LAS X v3.5.7.23225. SoftMax Pro 6.2 plate reader software. Flow Jo v10.8.1 GraphPad prism 10.0.2. Fiji ImageJ1.53t, Imaris image analysis, Ncounter data analysis Rosalind bio, Isospeak software. Living Image@Version 4.7 Software. Illustration images in FIGS. 2g and 5a were made using biorender.

The results of the experiments are now described.

### Example 1: Improvement of Cryopreserved NK Cell Viability and Function with Cytokine Pre-Treatment

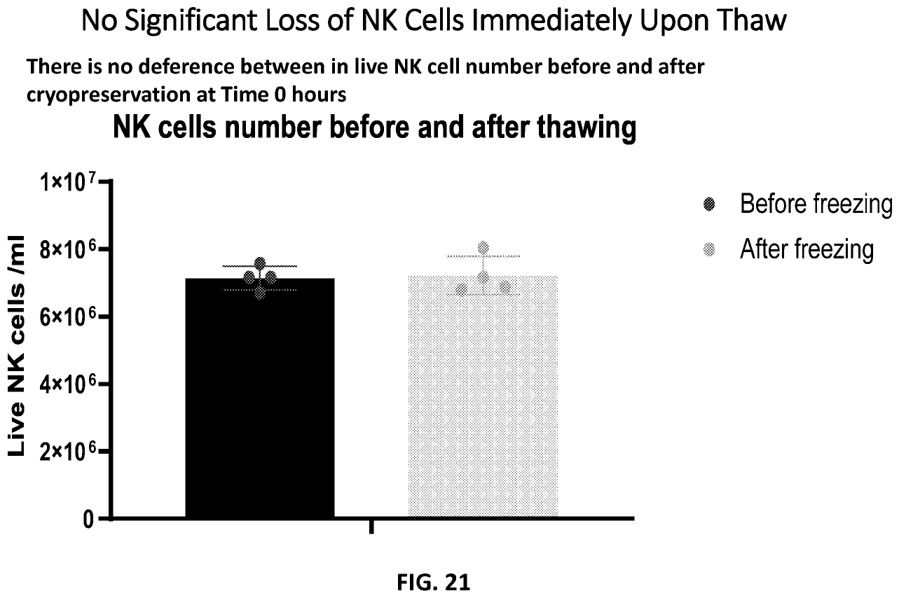

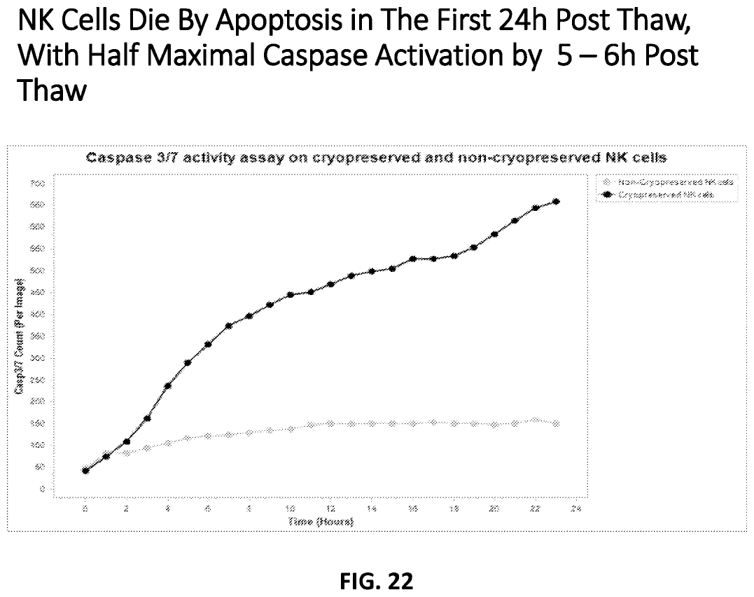

NK cells are cytotoxic lymphocytes that can kill virally infected cells or tumor cells. There is a growing clinical interest in using NK cells as cellular therapies to treat a variety of diseases including cancers. To make commercially available NK-based cellular therapies, large numbers of NK cells would be needed to be produced and then cryopreserved until use. Unfortunately, NK cells do not cryopreserve well. Indeed, FIG. 21 illustrates that while there is little cell loss immediately following thawing, observations of caspase 3/7 activation taken over the next 24 hours reveal that the significant cell loss over this time period is due to apoptosis (FIG. 22). Further, not only is viability poor 24 hours after thaw, but also NK cells that do manage to survive often display significant functional impairment, as illustrated in FIG. 2. Here, NK cells were thawed and rested in NK medium for 24 hours prior to use in a cytotoxicity assay using Raji 02M KO cells transduced with click beetle green (CBG). Significant loss in NK cell cytotoxicity is seen after thawing.

The studies disclosed herein demonstrate a cytokine signaling pathway involving IL-18, which helps improve NK cells recovery and function after standard methods of cryopreservation.

Cytokines play an essential role in NK cell development, maturation and activation. Herein, and without wishing to be bound by theory, it was hypothesized that pre-treating isolated NK cell with cytokines before cryopreservation would help improve NK cell recovery and function. As disclosed herein, studies found that engaging the IL-18 receptor using recombinant IL-18 significantly improved NK cell recovery and function. Adding IL-15 also helps NK cells recovery but engaging the IL-18 pathway was consistently better. IL-12 is a pro-inflammatory cytokine that activates NK cells. Conversely, studies disclosed herein found that adding IL-12 to NK cells consistently and significantly reduced NK cell recovery and function after cryopreservation.

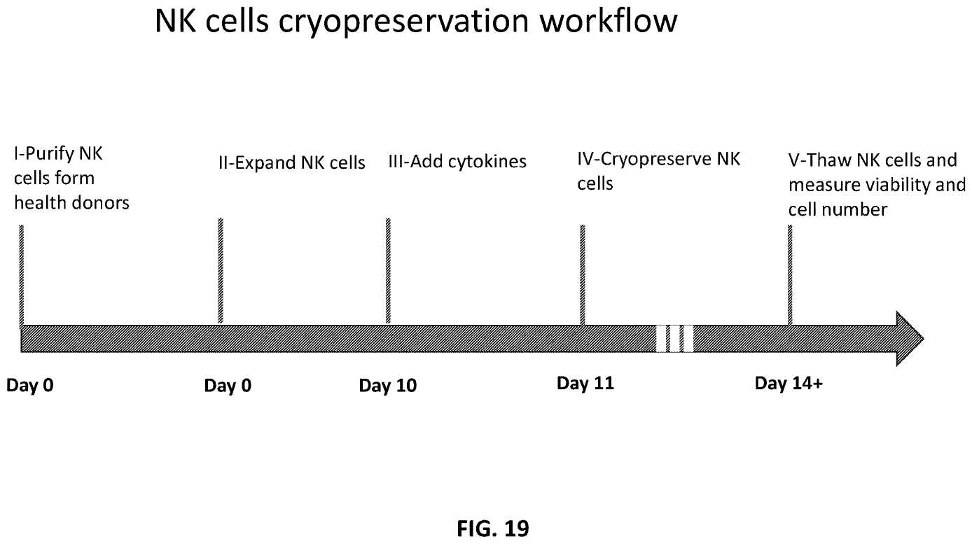

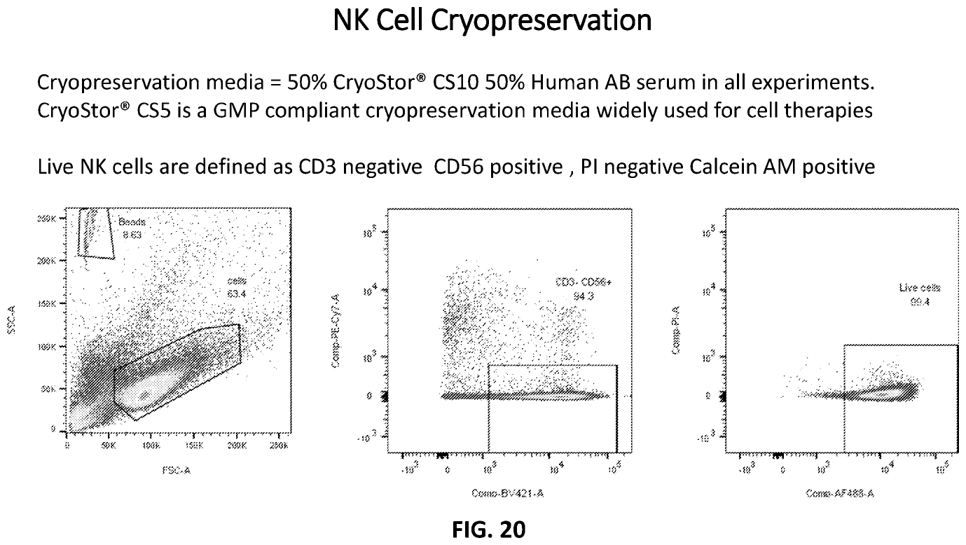

FIG. 19 illustrates a workflow for the cryopreservation of NK cells with cytokine pretreatment disclosed herein. Here, NK cells were purified from healthy donors then expanded ex vivo for 10 days using irradiated K562 cells which had been engineered to secrete mouse IL-21 (mIL-21) in order to promote NK cell expansion. Expanded NK cells were then washed once with complete NK cells media and resuspended in 2 ml of media at 2×10⁶ NK cells/mL. Recombinant IL-12, IL-15 and IL-18 were then added individually to the appropriate concentration and the cells were cultured for an additional 24 hours. The cells were then washed and resuspended in 1 ml of GMP cryopreservation media (CS5) and cooled to −80° C. overnight in a cryopreservation chamber (cooling rate is 1° C./min). The frozen cells were then stored in liquid nitrogen (liN2) for at least 72 hours prior to thawing, recovery, and use in subsequent experiments.

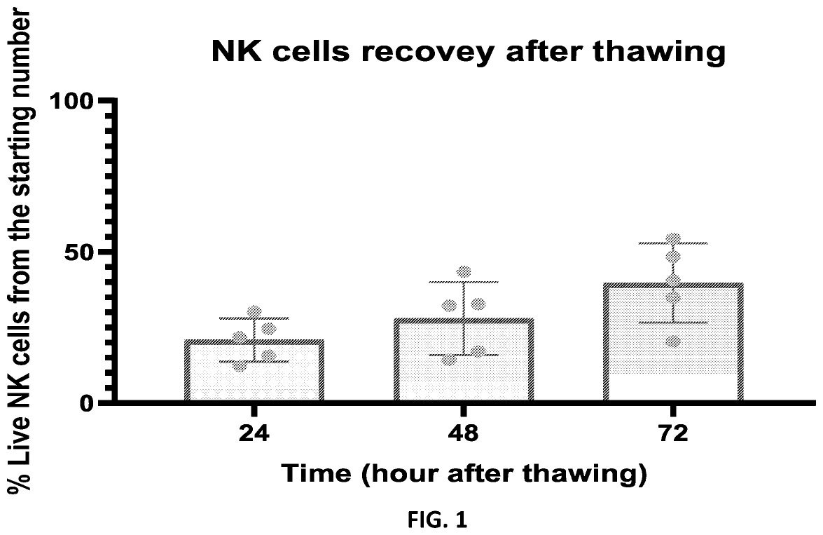

FIG. 1 illustrates NK cell recovery after thawing. For this study, NK cells were cryopreserved using CryoStor® CS5 freezing medium using a controlled rate freezer chamber placed in an −80° C. freezer. After 10-14 hours cells were moved to liquid nitrogen freezer. Three to several days later, frozen cells were thawed in a 37° C. water bath until small piece of ice was observed to be left. The thawed cells were then suspended in NK media (Lonza X-vivo 10, 10% Human AB serum, 100 U/mL IL-2) and counted by flow cytometry. Significant cell loss after is seen by 24 h post thaw, after which the cells start to proliferate and numbers recover, though still below 50% of the amount cryopreserved even after 72 h culture with IL-2. Flow cytometry stains were used to identify NK cells by using CD56⁺ and CD3⁻ as markers. For viability staining, Calcein-AM and propidium iodide (PI) used to differentiate live from dead cells. Counting beads were used to quantify NK cells. See FIG. 2

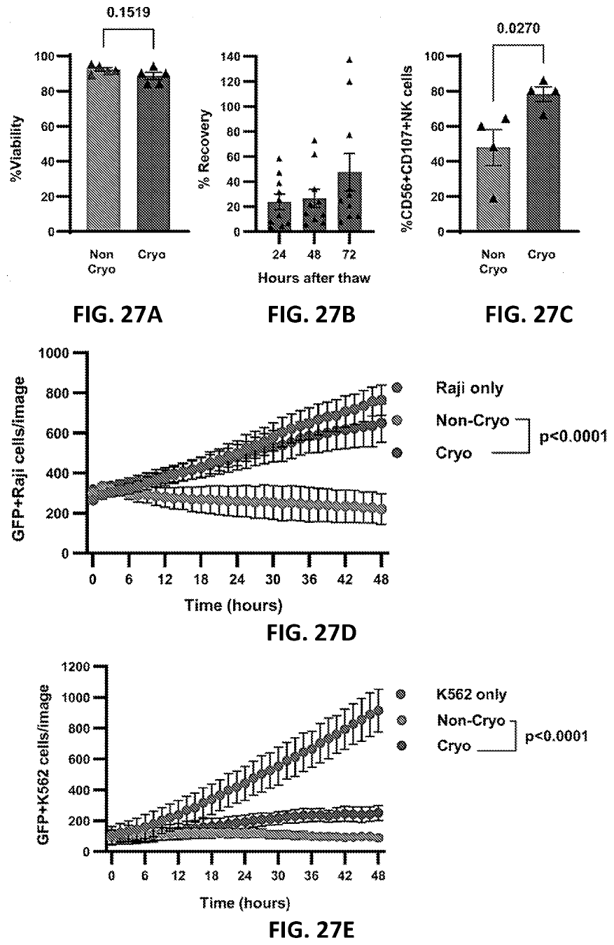

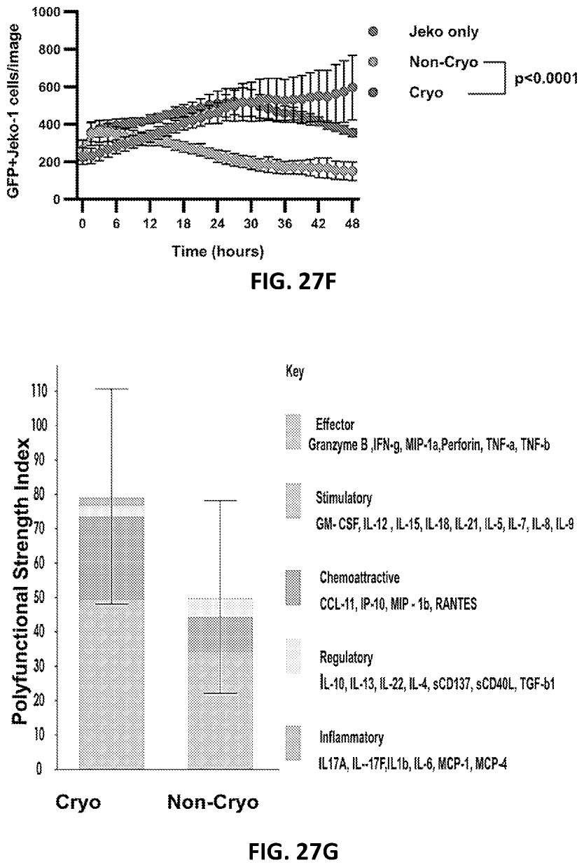

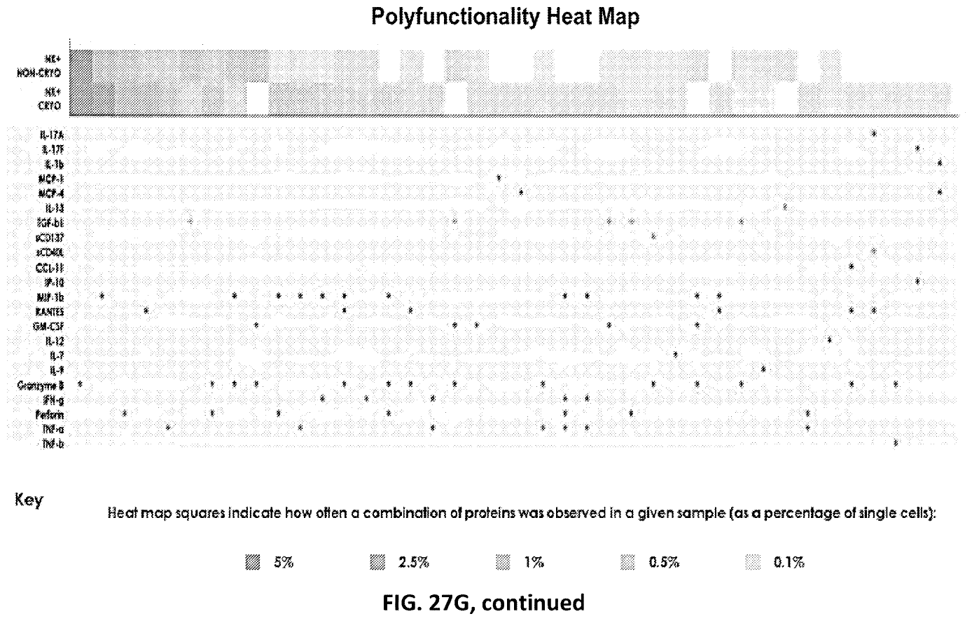

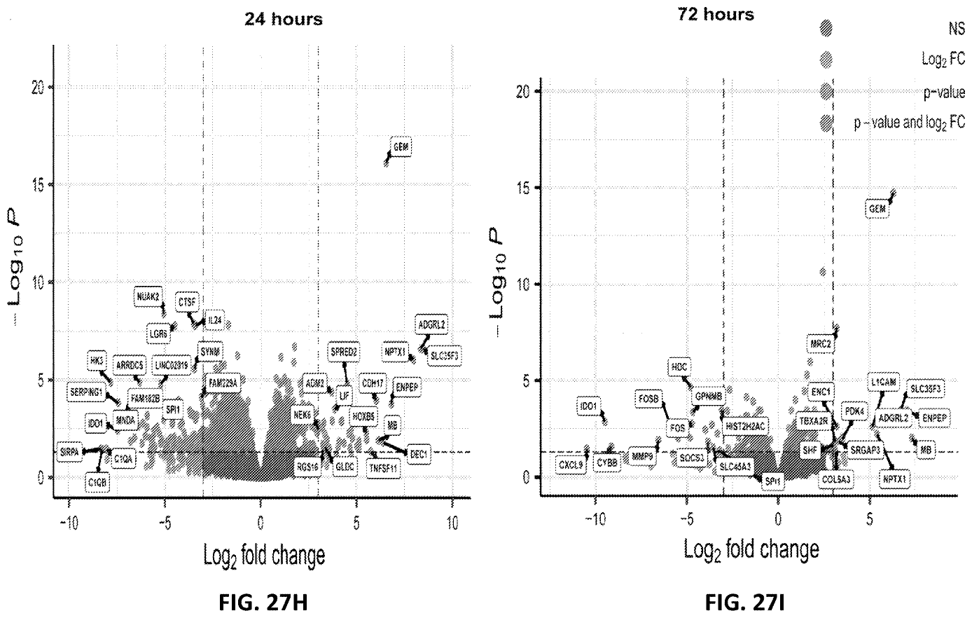

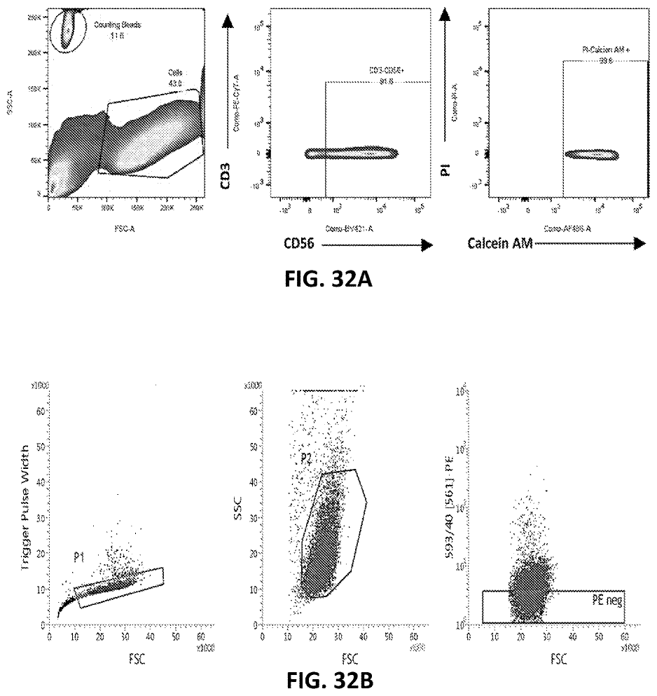

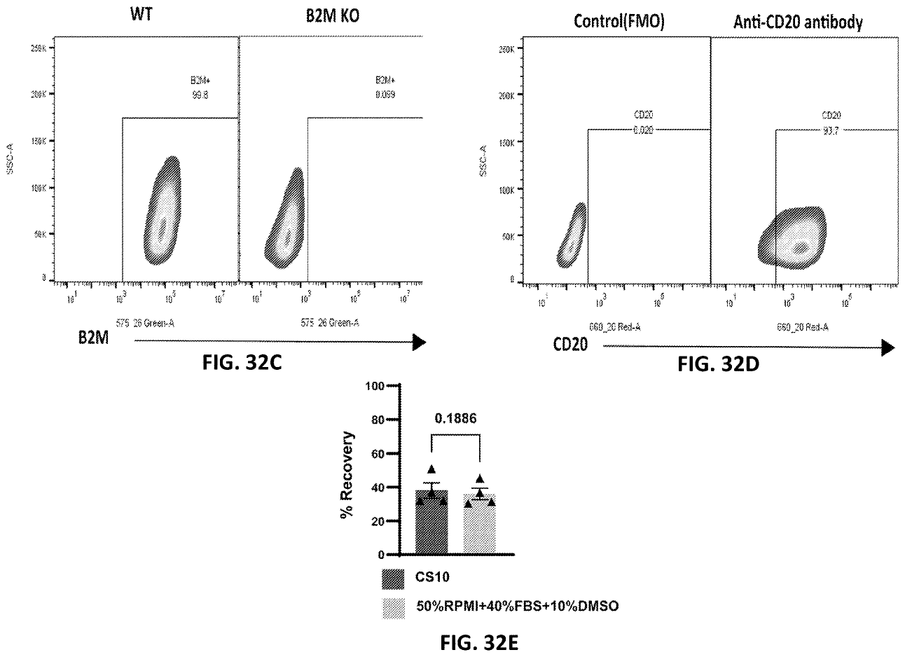

In a similar study, the effects that cryopreservation has on the recovery and function of EA NK cells were characterized using GMP-compliant cryomedia (CryoStor 5® or 10®) and the research scale, controlled rate CoolCell® cell freezer. The viability of NK cells before and after cryopreservation was measured (FIG. 27A) using flow cytometry with antibodies and viability dyes. Consistent with prior findings, drastic changes in NK cell viability were not observed, which was due to short-term assessments and experimental bias stemming from the use of flow cytometry gating strategies that exclude dead cells (FIG. 27A). In a time, course after thawing, a drastic loss of NK cell numbers by 24 hours post-thaw was found compared to immediately after thaw (time 0 hours) (FIG. 27B). Next, it was assessed whether the NK cells that survived cryopreservation maintained their effector functions. Degranulation and cytotoxicity assays were performed 24 hours after thawing NK cells using either K562 or B2M −/− Raji cells (FIGS. 32B, 32C) (FIGS. 27C. 27D, 27E) as target cells. Surprisingly, cryopreserved cells had higher surface expression of the degranulation marker CD107a than non-cryopreserved NK cells when cultured with K562 cells (FIG. 27C), however we found that cryopreserved NK cells were significantly less cytotoxic (FIGS. 27D, 27E). The ability of the non-cryopreserved and cryopreserved NK cells to mediate ADCC against the CD20+ mantle cell lymphoma (MCL) cell line Jeko-1 was also investigated (FIG. 32D). Cryopreserved NK cells showed a significant loss of ADCC compared to non-cryopreserved NK cells (FIG. 27F). The impact of cryopreservation on the single-cell cytokine secretome of NK cells in response to R848, an agonist of Toll-like receptors (TLRs) 7 and 8 was also assessed. Interestingly, cryopreserved NK cells had a higher Polyfunctional Strength Index (PSI), a product of the number of cells secreting more than one cytokine and the intensity of the signal for each secreted factor (FIG. 27G), which agrees with their increased CD107A expression (FIG. 27C).

Next, the effect of cryopreservation on the NK cell transcriptome at 0-, 24-, and 72-hours post-thaw was investigated. Via differential expression analysis it was observed that 153 and 209 genes were differentially expressed at 24- and 72-hours post-thaw respectively (Padj>0.05). Interestingly, it was observed that trends of downregulation of chemokine receptors and ligands (FIG. 27H), which concurs with reported findings of defects in NK cell motility post-thaw. Gene Set Enrichment Analysis (GSEA) revealed the IL-2-induced STAT5 pathway, which is critical for NK cell activation and survival, was upregulated post-thaw (Padj 1.1·10-5) (FIG. 27H). The RNA sequencing analysis, together with the degranulation and cytokines secretion data suggested that either the cryopreservation and thawing process activates NK cells or those that are activated become enriched through survival or proliferative benefits post-thaw. Lastly, an NK cell cryopreservation protocol was tested using cryomedia that includes 40% serum, which has been reported to enhance NK cell recovery but found EA NK cells from the expansion protocol showed no improvement in recovery using this serum-rich cryomedia compared to CryoStore 10 (FIG. 32E). In sum, these data concur with prior reports of a substantial loss of NK cell numbers and cytotoxic function following cryopreservation. We further found that the NK cells that survive cryopreservation have enhanced secretory activity.

GZMB Leakage Induces Apoptosis in NK Cells after Cryopreservation

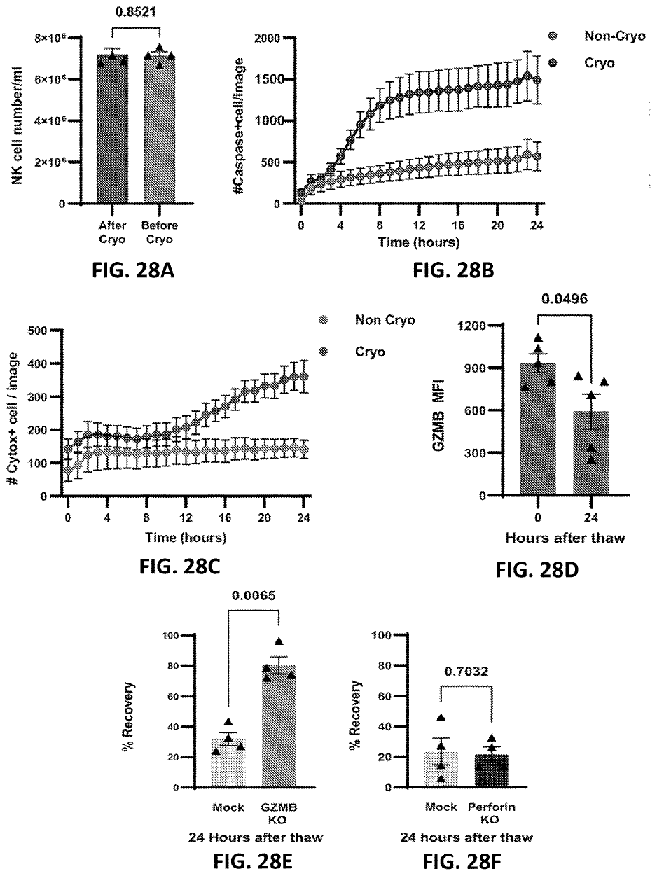

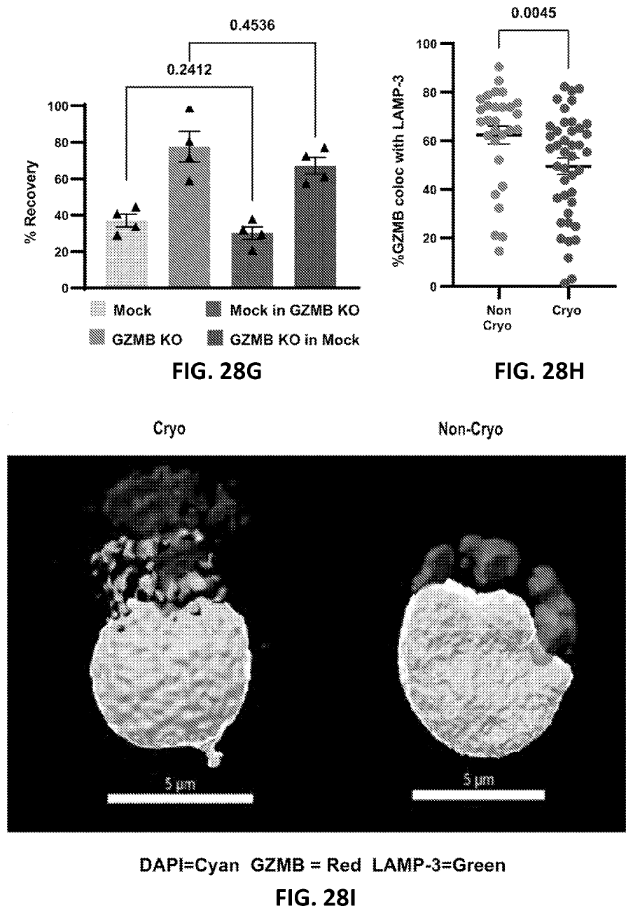

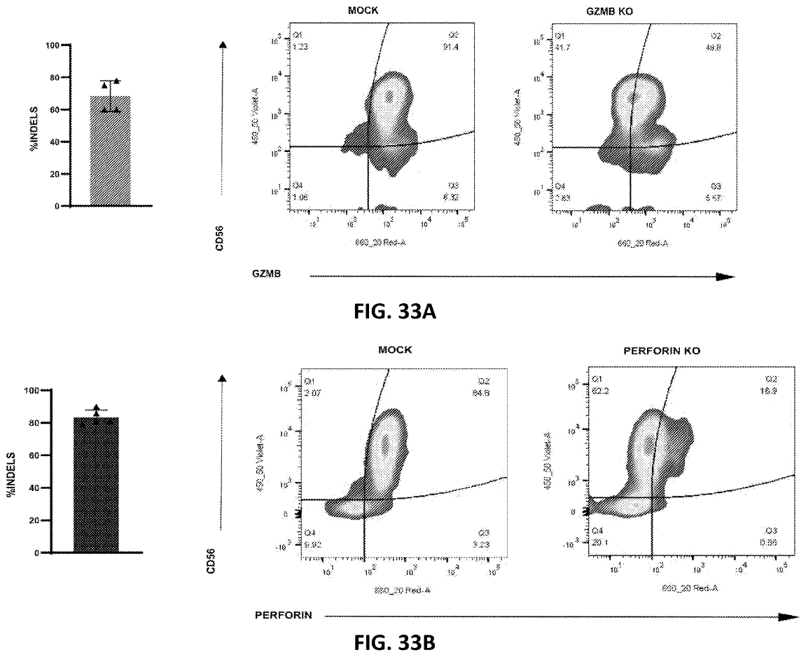

Cryoinjury largely results from the formation of ice crystals that disrupt cellular structures. Gross structural damage to NK cells might cause necrosis, although this is unlikely given the use of DMSO as a cryoprotectant and prior data as well as data presented herein (FIGS. 27A, 27B) suggesting that NK cells are not lysed by cryopreservation but die later by an active process. Such an active process might be the induction of apoptosis either directly or via fratricide. To investigate, NK cell numbers were measured before cryopreservation and at time 0 hours after thawing and found no significant cell loss immediately after thawing (FIG. 28A), ruling out immediate necrosis. Next, in a time-course assay, caspase 3/7 activation and staining with a vital dye to assess early apoptosis and cell membrane integrity was measured, respectively. Caspase 3/7 activation was observed in cryopreserved NK cells above the levels seen in non-cryopreserved NK cells from 4 hours post-thaw and rose rapidly before reaching a plateau after 9 hours (FIG. 28B). After 12 hours post-thaw, vital dye uptake was measured, indicative of cell death, which increased through the remainder of the time-course (FIG. 28C). These experiments confirmed the expected scenario supported by prior evidence that cryopreserved NK cells go through programmed cell death after thawing and are not necrotic due to acute physical injury. Considering the stark differences in cryopreservation tolerance between CD8+ T cells and NK cells, despite both being cytotoxic lymphocytes, the greater granularity of NK cells due to high levels of preformed cytotoxic granules and their unique ability to recognize cell stress emerge as key points of differentiation. In this context, apoptosis in cryopreserved, thawed NK cells might conceivably be due to autolysis if cryoinjury results in intracellular GZMB leakage from preformed cytotoxic granules, or due to fratricide following the induction of cellular stress and its recognition by neighboring NK cells. Since cryopreserved NK cells have reduced cytotoxicity but increased CD107A expression, we measured intracellular levels of GZMB and found a reduction in intracellular GZMB levels 24 hours post-thaw (FIG. 28D), suggesting selective survival of GZMB^{Lo} cells. However, this finding does not rule out the fratricide hypotheses because surviving NK cells may have recently degranulated (CD107A+GZMB^{Lo}). To confirm the role of GZMB in the post-thaw apoptotic cell death pathway of NK cells, CRISPR-Cas9 editing was conducted to knockout (KO) GZMB and confirmed KO on the DNA and protein levels (FIG. 33A). GZMB KO NK cells were next cryopreserved and thawed and a significant improvement in the numbers recovered post-thaw was observed (FIG. 28E).

To address whether NK cells are dying by fratricide or autolysis, CRISPR-Cas9 editing on PRF1 (perforin) was performed and confirmed perform KO on DNA and protein levels (FIG. 33B). PRF1 KO NK cells had similarly poor NK cell recovery after cryopreservation and thawing compared to mock KO NK cells (FIG. 28F), suggesting that GZMB is acting in a perforin-independent manner, favoring the autolysis hypothesis over fratricide. As there are reports that GZMB can kill target cells in a perforin-independent manner, GZMB KO cells were labeled with CFSE after thawing and cocultured with an excess of unlabeled mock KO NK cells at an effector to target (E:T) ratio of 1:10, and the reverse was also performed by CFSE labeling mock KO NK cells and coculturing them with an excess of unlabeled GZMB KO NK cells at the same E:T ratio (FIG. 28G). Without wishing to be bound by theory, it was hypothesized that if cells were dying of fratricide, a reduction in the recovery of GZMB KO NK cells when cocultured with mock KO NK cells should be observed because the mock KO NK cells have intact GZMB. Similarly, mock KO NK cells should have a better recovery when cocultured with GZMB KO NK cells because fratricide would be largely dependent upon encountering other GZMB-intact mock KO NK cells, which are present at low proportions. The recovery of GZMB KO NK cells co-cultured with an excess of mock KO NK cells and 350 351 mock KO NK cells cocultured with an excess of GZMB KO NK cells was unaffected (FIG. 28H) confirming that fratricide does not play a meaningful role in NK cell apoptosis post-thaw, which instead is potentially the result of autolysis.

Having identified that autolysis is the most likely cause of NK cell apoptosis post-thaw, we examined whether GZMB leaks from cytotoxic vesicles post-thaw using confocal microscopy via staining for GZMB and the vesicle marker LAMP-3. Decreased GZMB co-localized with LAMP-3 was observed in cryopreserved NK cells compared to non-cryopreserved NK cells (FIGS. 28I, 28J), supporting GZMB leakage from preformed cytotoxic vesicles. Together, these data show that NK cells cryopreserved in GMP-compliant cryomedia containing 5 or 10% DMSO and using a controlled rate freezer undergo programmed GZMB-dependent autolysis in the 24 h following thawing subsequent to the leakage of GZMB from cytotoxic vesicles. Neither necrosis nor fratricide appear to play a significant role.

IL-15+IL-18 Pretreatment Improves NK Cell Recovery and Function after Cryopreservation by Upregulating Anti-Apoptotic Genes and Temporarily Reducing Intracellular GZMB Levels Via Degranulation

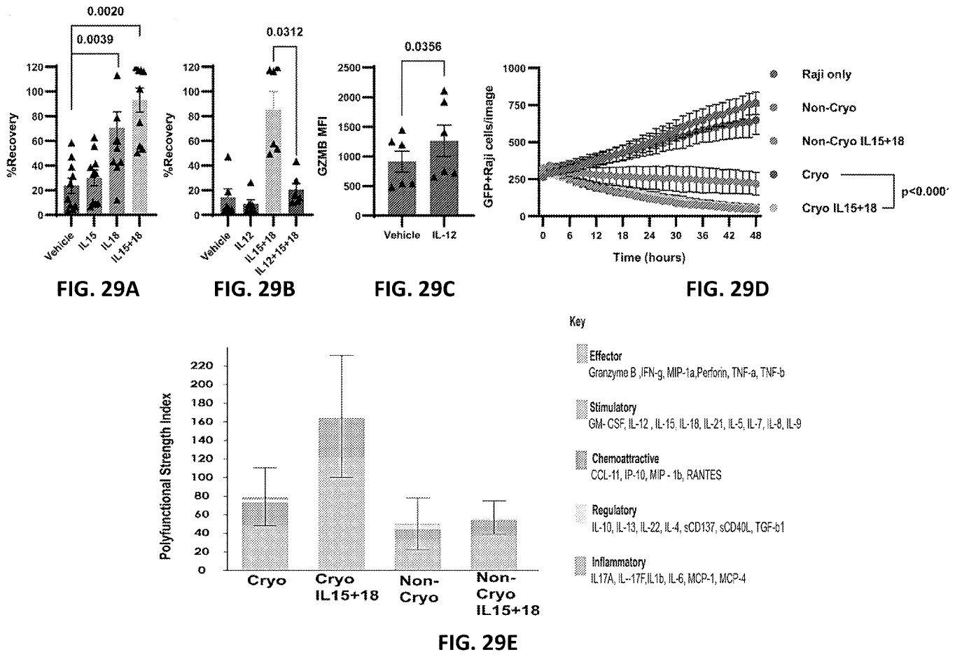

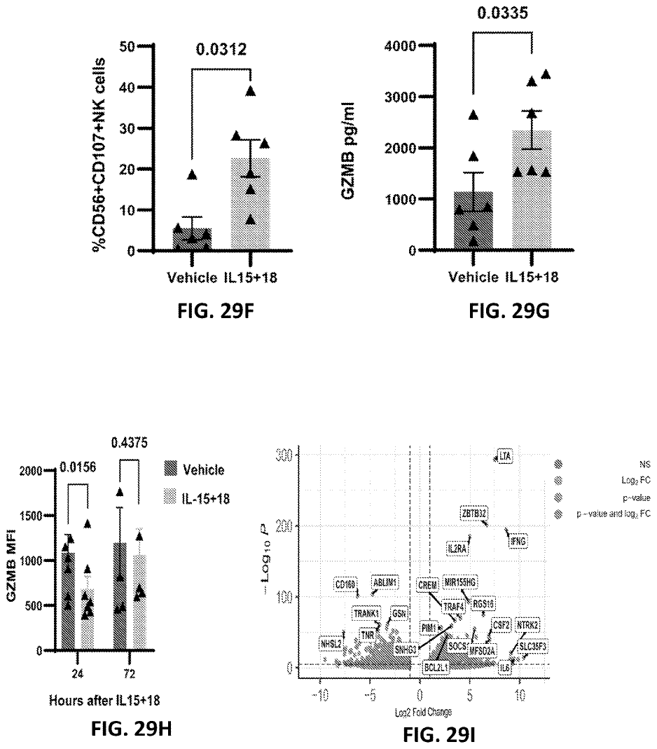

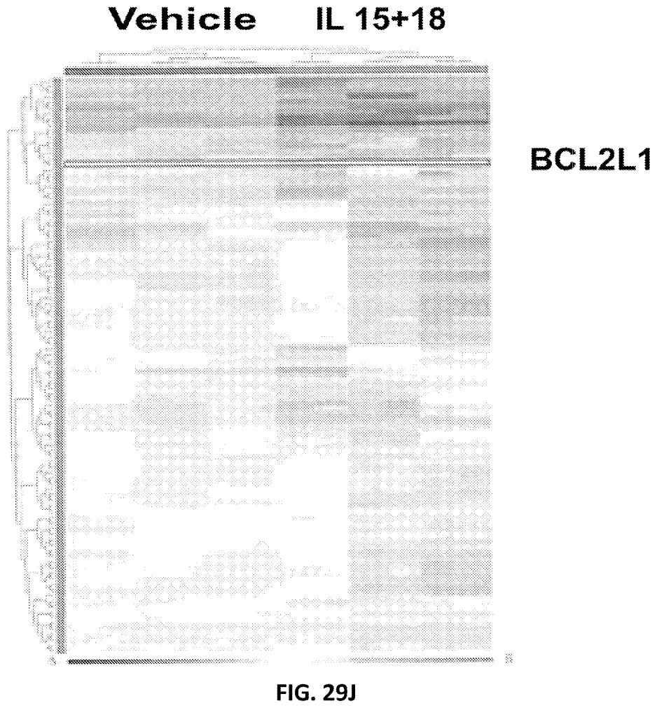

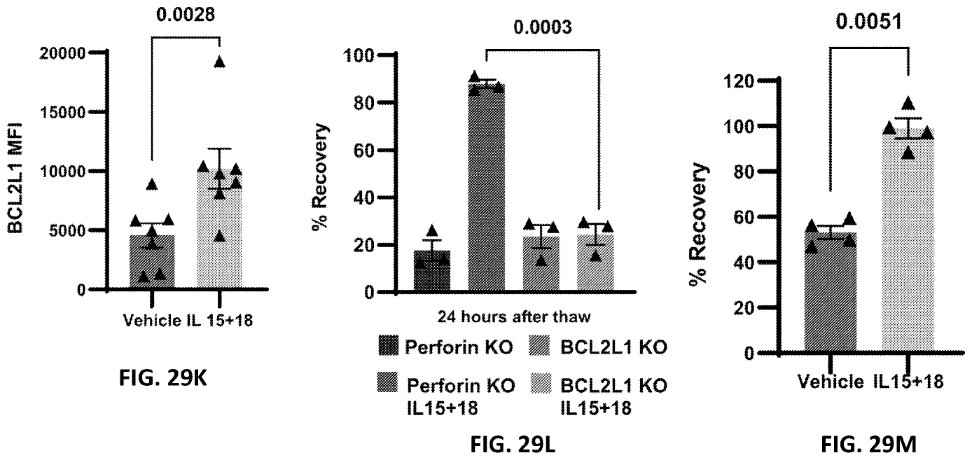

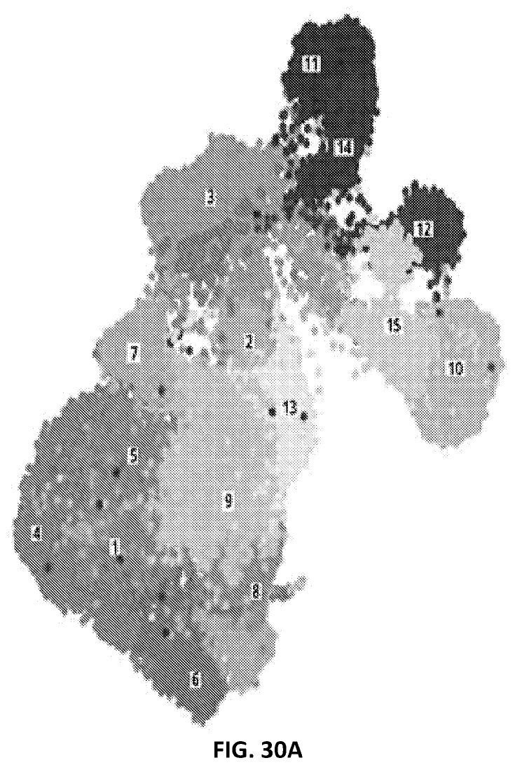

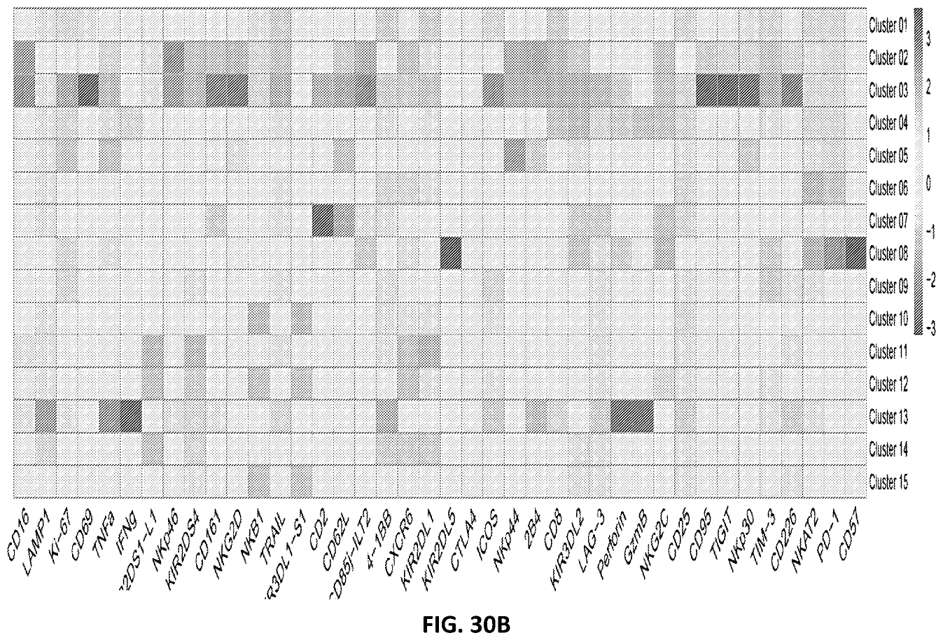

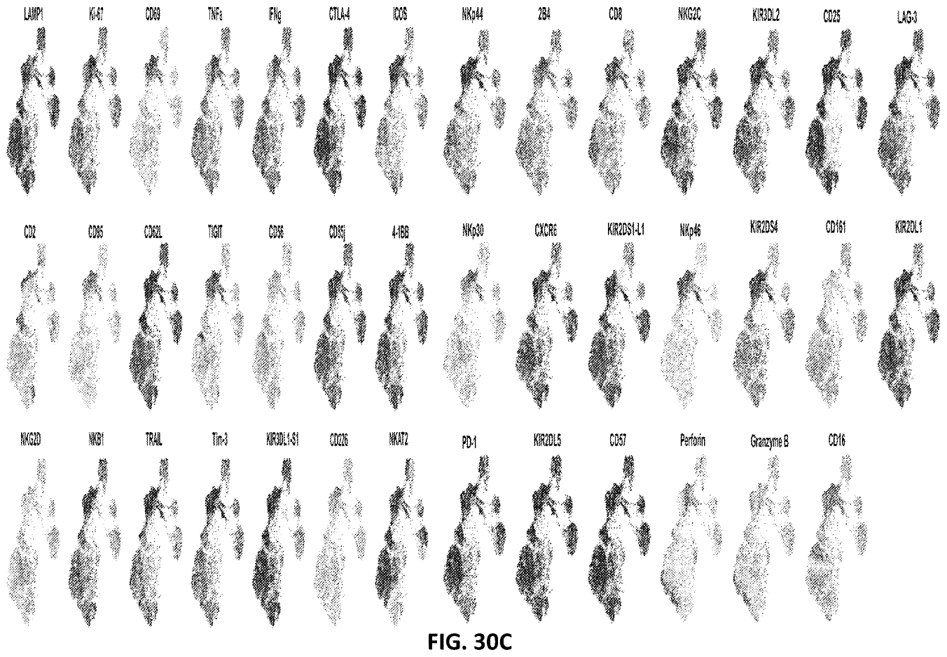

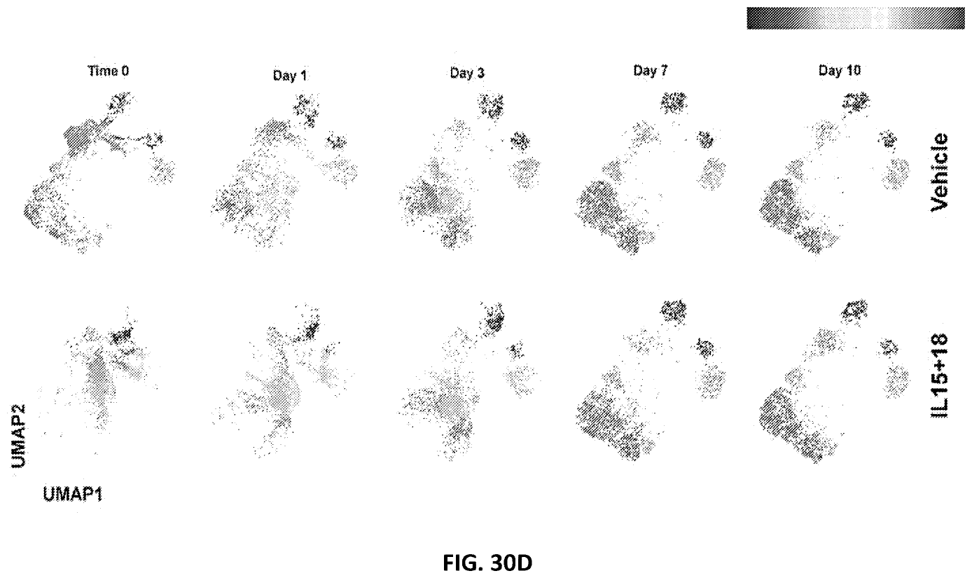

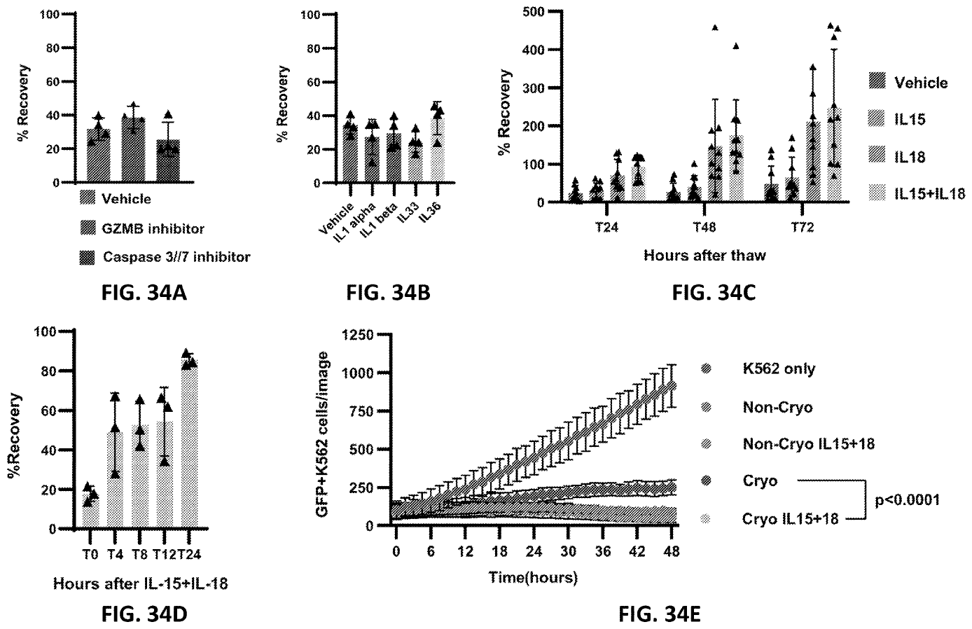

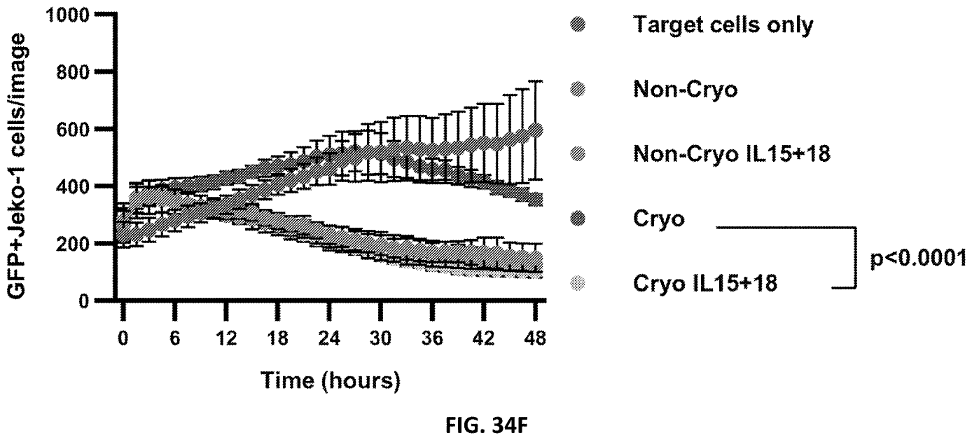

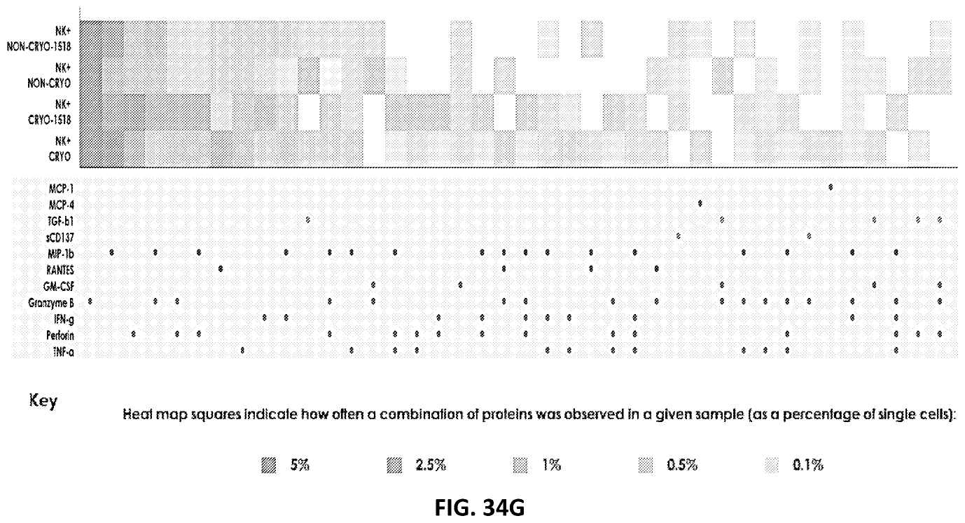

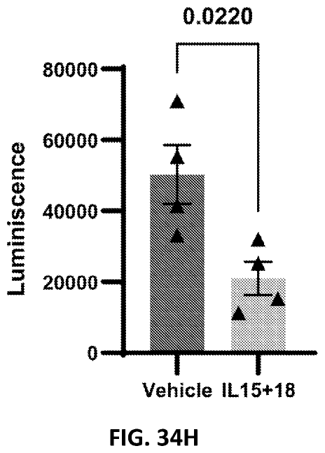

Having established that the loss of NK cell viability and function after cryopreservation is caused by GZMB-mediated autolysis rather than fratricide or necrotic cell death, interventions that could be applied prior to cryopreservation to enhance NK cell recovery by mitigating the effects of GZMB leakage were explored. First, a small molecule reversible inhibitor of GZMB and an irreversible pan caspase inhibitor were tested, but neither compound improved the recovery of NK cells post thaw (FIG. 34A). In both cases, it is possible the compounds did not adequately saturate the GZMB-caspase cell death pathway, especially given the likely protein turnover in the first 12-24 hours post thaw, and for the reversible GZMB inhibitor, the compound was likely washed from the NK cells post thaw and/or diluted out in the fresh culture medium. Cytokines were next screened, which represent a potentially ideal solution for both scientific and practical reasons. Indeed, cytokines including IL-2, -15, and -18 have been shown to induce anti-apoptotic pathways, with IL-18 in particular protecting NK cells from various inducers of apoptosis. Moreover, in terms of practicality, GMP-grade cytokines are commercially available, readily integrated into NK cell manufacturing processes, and do not need to be present in the final cell product since their effects are indirect via receptor activation. Given prior data on IL-18 and the use of IL-12, -15, and -18 to produce cytokine-induced memory-like (CIML) NK cells, a screen of the IL-1 family members (IL-1a, -1b, -18, -33, and -36), IL-12, and -15 was conducted. These studies did not include IL-2 or IL-21 in the screen because the NK cells were already expanded with both cytokines and were maintained in IL-2-supplemented media. It was found that pretreating NK cells with IL-18 alone significantly improved their recovery post-thaw, while IL-12 pretreatment reduced recovery (FIGS. 29A, 29B). Pairwise and triple combinations of IL-12. -15, and -18 were tested and determined that NK cells pretreated with the IL-15+IL-18 combination 24 hours before cryopreservation had optimal recovery, while all combinations, including IL-12, showed no enhancement of recovery (FIGS. 29A, 29B, 34C). Next, NK cells were treated with IL-15+/IL-18 for 0, 4, 8, 12, and 24 hours before cryopreservation to establish the optimal timepoint for cytokine pretreatment. A significant improvement in recovery was found by 4 hours of exposure to IL-15+IL-18, which did not improve significantly with up to 12 hours of exposure, but was maximal at 24 hours of exposure prior to cryopreservation, suggesting a mechanism that includes both rapid (<4 hours) and slow (>12 hours but ≤24 hours) components (FIG. 34D).

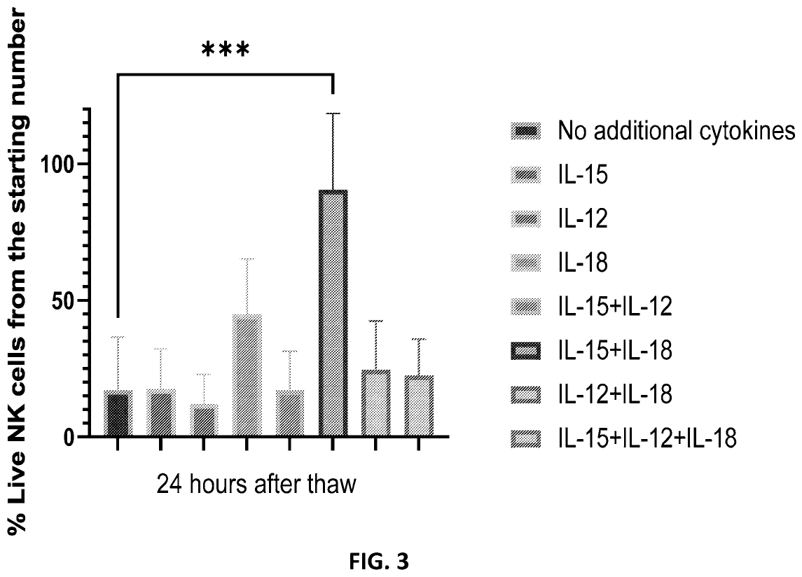

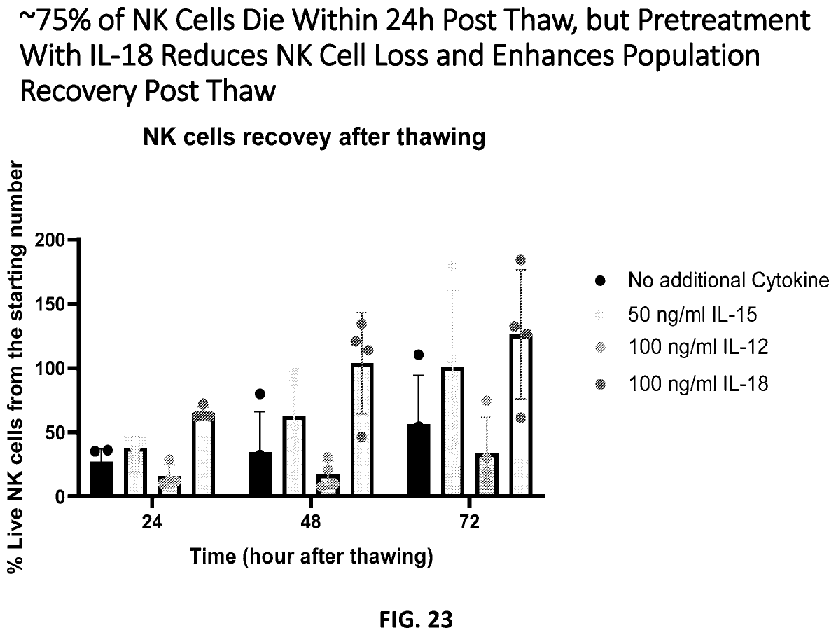

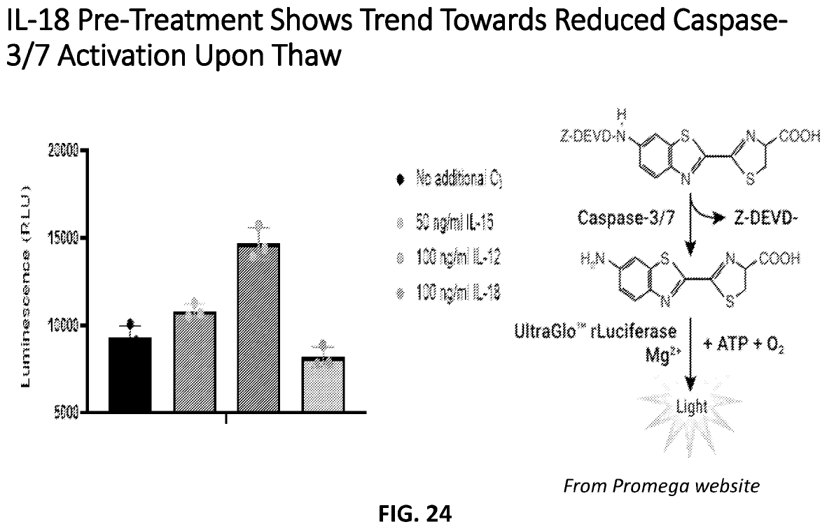

In order to further determine whether pre-treatment of NK cells with various cytokines could improve thawed NK cell viability and function, NK cells from four heathy donors were expanded for 10 days and then treated with IL-12 100 ng/ml, IL-15 50 ng/ml, IL-18 200 ng/ml, IL-12 100 ng/ml+IL-15 50 ng/ml, IL-12 100 ng/ml+IL-18 200 ng/ml, IL-15 50 ng/ml+IL-18 200 ng/ml, IL-18 200 ng/ml+IL-12 100 ng/ml or IL-12 100 ng/ml+IL-18 200 ng/ml+IL-15 50 ng/ml for 24 hours prior to cryopreservation in CS5. Cells were thawed and live cell number was measured after 24 hours (FIG. 3) or after 24, 48, and 72 hours (FIG. 23). IL-18+IL-15 significantly improved NK cells recovery after cryopreservation. Although IL-18 alone also improved NK cell recovery after cryopreservation. We saw a trend that IL-15+IL-18 is better. (Unpaired t-test p=0.0009 for untreated versus the combination of IL-15+IL-18). A Caspase-Glo® 3/7 Assay was preformed according to the manufacturer's protocol on NK cells after thawing and found reduced Caspase-3/7 activation in NK cells pretreated with IL-18 (FIG. 24).

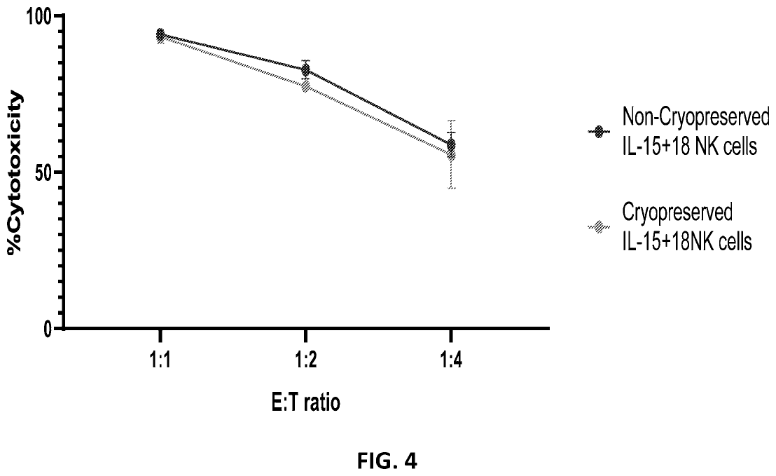

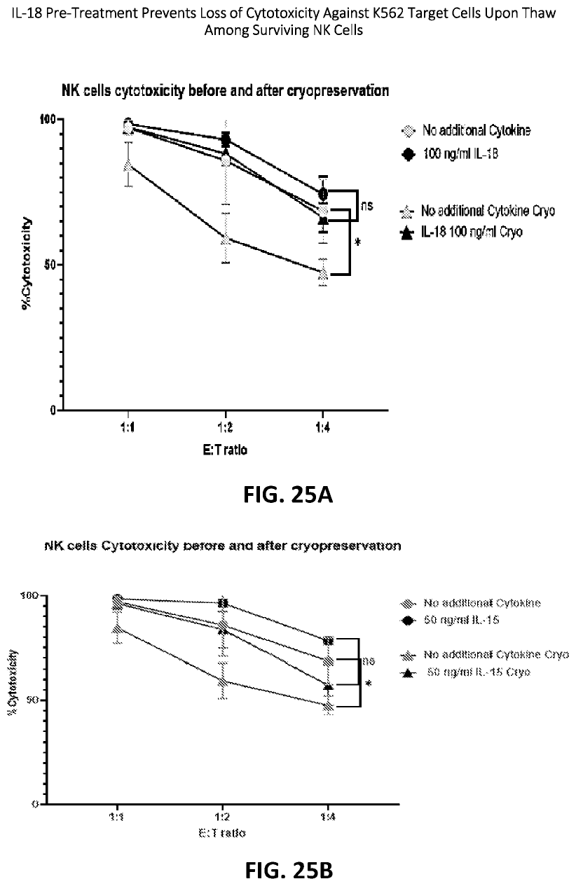

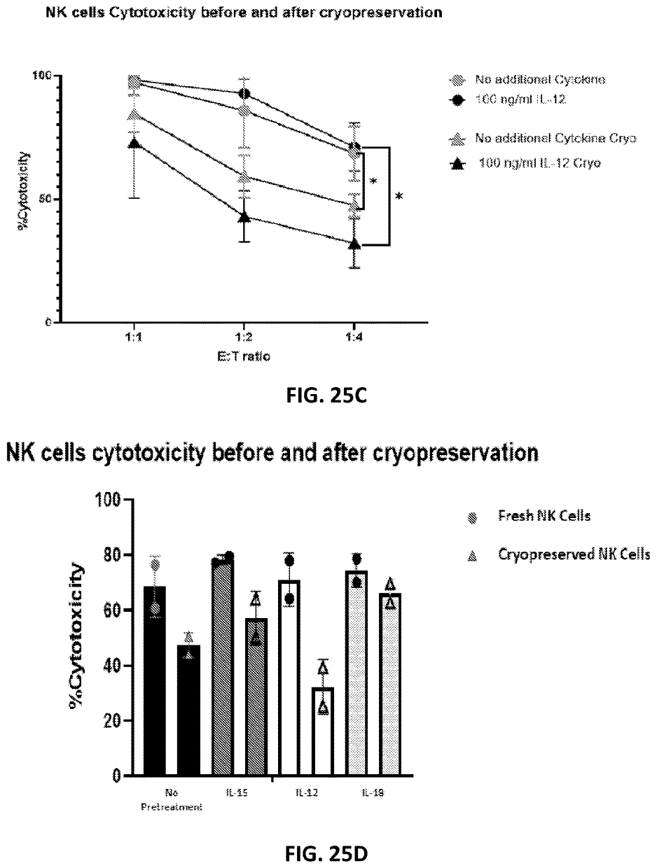

A series of studies was then undertaken in order to determine whether IL-18 or IL-15+IL-18 pre-treatment could maintain the cytotoxic function of NK cells. In FIGS. 25A-25D, NK cell cytotoxicity assays were performed using a luciferase-expressing K562 cell line. Non-cryopreserved and cryopreserved NK cells were co-cultured with target cells for 24 hours at Effector:Target ratios of 1:1, 1:2, and 1:4. Luciferase substrate was added for 15 minutes at room temperature. Luminescence was read using a plate reader. This assay shows that in addition to preserving NK cell viability IL-18 pretreatment preserves NK cytotoxicity with the least difference between fresh and cryopreserved NK cells compared to all other groups. The assay in FIG. 4 included the IL-15+IL-18 combination treatment of NK cells, which were thawed and rested for 24 hours before using in a cytotoxicity assay with CBG-expressing 02M KO Raji cells. Here, the combination pre-treatment resulted in no significant loss of NK cell cytotoxicity after thawing.

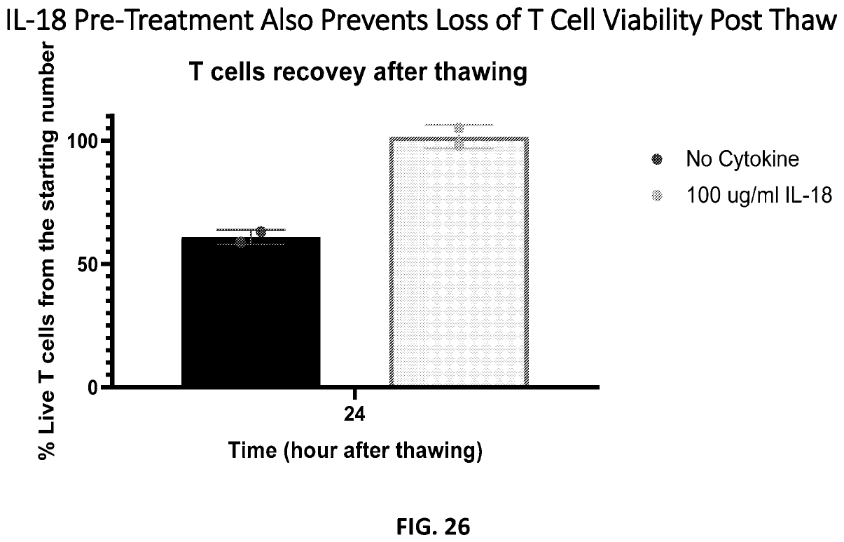

In order to determine whether IL-18 pre-treatment could preserve T cells as well as NK cells, T cells were isolated from health donors and were expanded using CD3-CD28 beads for 10 days. T cells were then washed and cryopreserved under similar conditions to those used on NK cells in the studies presented herein. T Cell recovery was measured by viability stain and flow cytometry as mentioned elsewhere herein. FIG. 26 shows that in addition to preserving the viability of NK cells upon cryopreservation, IL-18 pretreatment also preserves the viability of T cells.

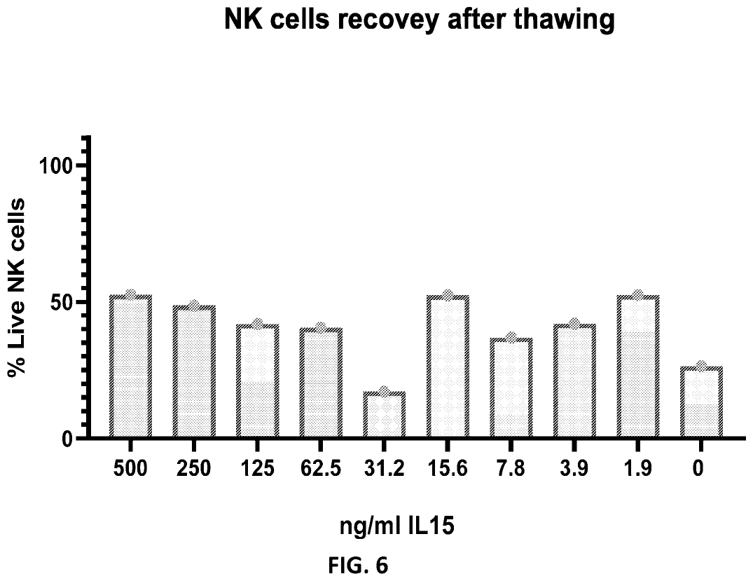

A series of studies were then conducted in order to determine the optimal concentration of IL-15 and IL-18 for NK cell pre-treatment. To determine the optimal dose of IL-18, NK cells were stimulated with a 2-fold dilution series of IL-18 from 500 ng/ml to 1.9 ng/ml prior to cryopreservation. After thawing, NK viability was assayed, and it was found that an IL-18 concentration ≥125 ng/ml was required to enable a high recovery rate (FIG. 5). Recovery improved further with greater concentrations of IL-18, although with diminishing returns in terms of additional recovery for each doubling of IL-18 concentration. To determine the optimal dose of IL-15, NK cells were stimulated with a 2-fold dilution series of IL-15 from 500 ng/ml to 1.9 ng/ml prior to cryopreservation. After thawing, NK viability was assayed, and it was found that IL-15 concentrations between 1.9 and 500 ng/mL did not impact NK cell viability in the absence of IL-18. These findings suggest that IL-15 potentiates the post cryopreservation viability benefit of IL-18 but does not have a benefit by itself (FIG. 6).

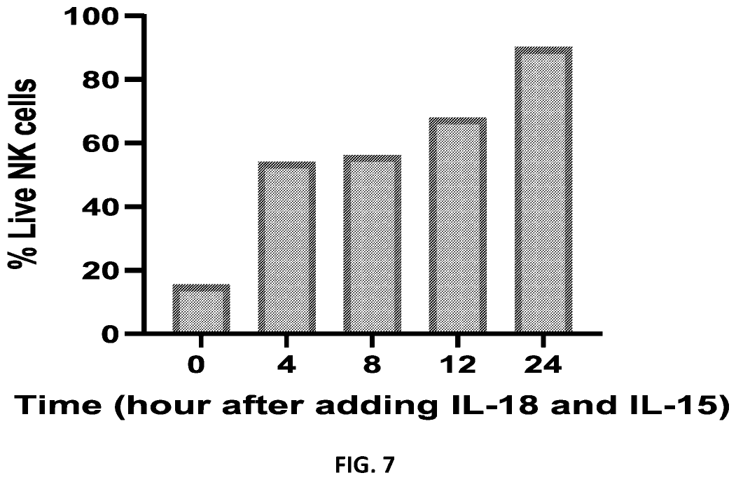

FIG. 7 illustrates a study to determine the optimal length of pre-treatment necessary to preserve NK viability after cryopreservation. Here, NK cells were stimulated with IL-15 and IL-18 for 0, 4, 8, 12 or 24 hours then cryopreserved using CryoStor CS5. Cells were thawed and live NK cells were counted using flow cytometry. A beneficial effect with >50% recovery was seen after as little as 4 h, but the maximal effect was not observed until 24 h suggesting it takes between 12 and 24 h for the combined effects of IL-15+IL-18 to occur.

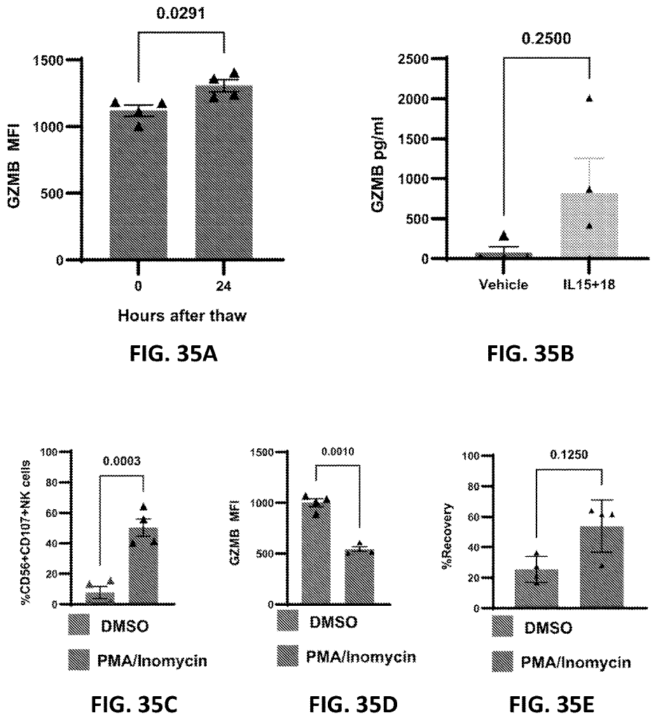

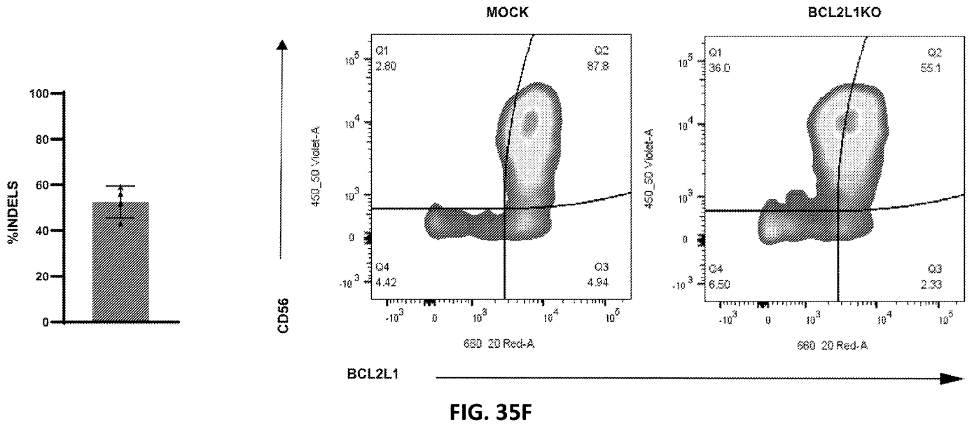

The profound negative impact that IL-12 had on NK cell cryopreservation was intriguing (FIG. 29B). To investigate, NK cells treated with vehicle or IL-12 and stained for GZMB, and it was found that IL-12 upregulates GZMB significantly (FIG. 29C), which likely explains the negative impact of IL-12 on recovery after the thawing. Next, the functionality of NK cells pretreated with IL-15+IL-18 was explored and their cytotoxicity and ADCC were found to be similar pre- and post-cryopreservation (FIGS. 29D, 34E, and 34F). Previous studies have shown that cryopreserved NK cells have higher polyfunctionality than non-cryopreserved NK cells (FIG. 27G), and this held true for IL-15+IL-18-pretreated cryopreserved NK cells (FIGS. 29E and 34G) but is no longer associated with reduced cytotoxicity. Caspase 3/7 activation was also measured in IL-15+IL-18-treated cells post-thawing and found that IL-15+IL-18-treated cells had caspase 3/7 activation signal above background but significantly lower than in vehicle-treated NK cells (FIG. 34H). IL-15 and IL-18 have multiple stimulatory effects on NK cells including the induction of degranulation and the upregulation of anti-apoptotic genes, both of which might both contribute to their cryoprotective effects. To explore degranulation. CD107a staining was examined and secreted GZMB in the media was quantified and found that IL-15+IL-18-treated NK cells reached peak degranulation after only 4 hours, leading to significant elevations of GZMB in the media (FIGS. 29F, 29G). Cell-associated GZMB levels were measured by flow cytometry at 24 and 72 hours after IL-15+IL-18 treatment and observed that a significant reduction in GZMB was seen at 24 but not 72 hours after IL-15+IL-18 (FIG. 29H), which is consistent with a temporary reduction by degranulation. To confirm that lower GZMB levels would not persist in IL-15+IL-18 pretreated NK cells post thaw, cell-associated GZMB levels were measured at 0- and 24-hours post-thaw, as well as secreted GZMB levels at 24 hours post-thaw, (FIGS. 34A and 34B). Increasing levels of cell-associated GZMB were found post thaw with the IL-15+IL-18 pretreated NK cells retaining a higher secretory phenotype than vehicle treated cells consistent with single cell secretome data (FIG. 29E). Based on these data, and without wishing to be bound by theory, it was hypothesized that reducing cell associated GZMB before cryopreservation might improve recovery after thawing. To test this, NK cells were treated with DMSO or Cell Activation Cocktail (PMA+Ionomycin). Degranulation and reduction in GZMB levels was confirmed in the PMA+Ionomycin treated cells and DMSO treated cells (FIGS. 35C and 35D). Then these cells were cryopreserved and thawed. Recovery of the PMA/Ionomycin treated cells trended towards improved recovery at levels similar to IL-15+IL-18 after 4 hours (FIGS. 34D, 35D) although they did not reach statistical significance (FIG. 35D).

To determine if IL-15+IL18 treatment induces the upregulation in genes that may protect NK cells from GZMB-initiated apoptosis, we performed a bulk RNAseq analysis on NK cells that had been treated with IL-15+IL-18 vs cells treated with vehicle-control. RNAseq data showed upregulation of the BCL2 gene family especially BCL2L1 (Bcl-XL) (FIG. 29I). To confirm this result, the NanoString nCounter assay was also used with a panel comprising probes to detect genes involved in apoptosis and found that BCL2L1 was one of the genes that is significantly upregulated (FIG. 29J). To confirm that BCL2L1 was upregulated on the protein level, intracellular staining was performed on NK cells treated with IL-15+IL-18 and a significant upregulation was detected (FIG. 29K). Finally, a CRISPR-Cas9 KO of BCL2L1 as performed and confirmed on the DNA and protein level (FIG. 35F) to determine whether BCL2L1 was necessary for the protective effects of IL-15+IL-18. The results showed profoundly diminished recovery of IL-15+IL-18-pretreated BCL2L1 KO NK cells in comparison to control gene KO (perform), highlighting that this anti-apoptotic factor is necessary to the mechanism for cryoprotection by IL-15+IL-18 (FIG. 29L). Lastly, a commercial scale controlled-rate freezer (the CryoMed™ controlled-rate freezer) was used to mimic a GMP cryopreservation process and found that IL-15+IL-18-treated NK cells showed 100% recovery of NK cells at 24 hours post-thaw (FIG. 29M). Together these data demonstrate that pretreatment with a combination of IL-15+IL-18 significantly and drastically improved NK cell recovery and function after cryopreservation. IL-15+IL-18 pretreatment protects NK cells by upregulating anti-apoptotic genes, specifically BCL2L1, which is necessary in rescuing NK cells from GZMB-initiated cell death and also by inducing degranulation, which temporarily reduces intracellular levels of GZMB and thus presumably the amount that may leak from preformed cytotoxic granules post-thaw. Temporal evidence supports this two-part mechanism since some improvement of recovery is seen within 4 hours of pretreatment, which is not improved upon until >12 hours but 24 hours of pretreatment, consistent with rapid degranulation followed by slower gene expression changes.

Deep Immuno-Profiling of Vehicle and IL-15+18-Treated NK Cells after Cryopreservation Using Cytometry by Time of Flight (CyTOF) Reveals Distinct but Reversible Changes in NK Cells Phenotype

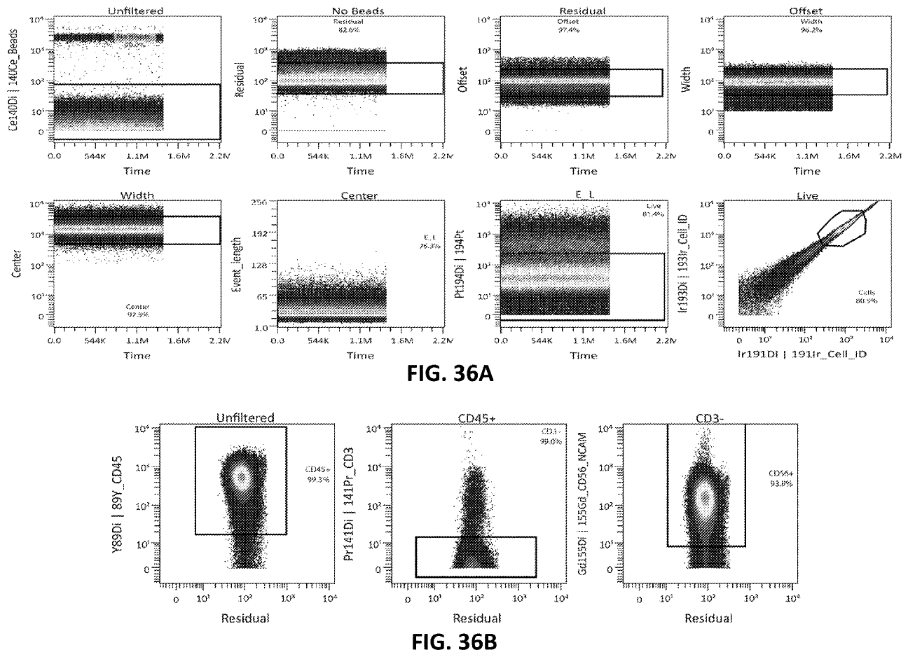

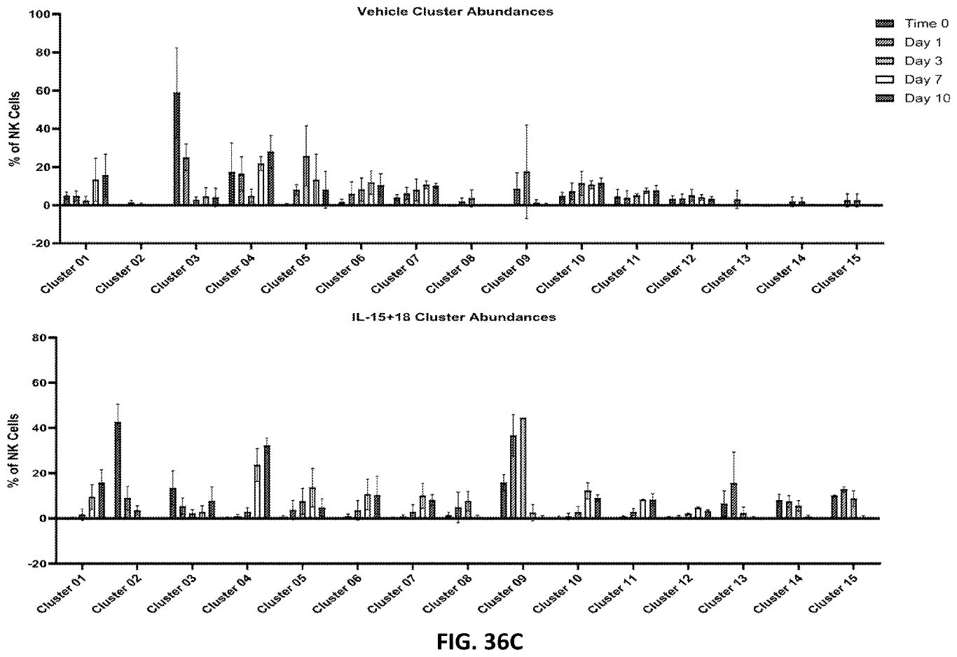

To explore the effect of IL-15+IL-18 treatment on the phenotype of NK cells a CyTOF panel comprised of 41 antibodies was used. Vehicle or IL-15+IL-18-treated NK cells were thawed and stained with viability dye and these antibodies in a 10-day time-course with analysis conducted on days 0, 1, 3, 7, and 10 post-thaw. Live, intact, single-cells were then gated to resolve total CD45+CD3-CD56+NK cells (FIGS. 36A and 36B) and identified a total of 15 unique clusters (FIGS. 31A and 31C). At time 0 after thawing most vehicle treated cells were in cluster 3 (FIG. 36C). This cluster is CD56^{dim}, CD69−, GZMB intermediate-high, whereas the majority of IL-15+IL-18-treated NK cells fell into 5 clusters: 2, 9, 13, 14 and 15. All these clusters had intermediate-high levels of CD25, 4-1BB, CD69, and LAMP-1. These data show that IL-15+IL-18 treatment activates NK cells in comparison to vehicle-treated cells as anticipated from the biological functions of these cytokines. At day 1 and 3 after thawing vehicle control and IL-15+IL-18-pretreated 674 675 NK cells began to show a converging trend with increases in clusters 5, 6, 9, 10, and 13 in vehicle-treated NK cells, with maintenance of clusters 9, 10, and 13 and increase in clusters, 5 and 6 in the IL-15+IL-18-treated NK cells. Cluster 5 is NKp30+, NKp44+. TNFα+ and Ki-67+, while cluster 6 has modest expression of a broad range of markers with high levels of KIR2DL3 and PD-1. By day 7 and 10 after thawing vehicle and IL-15+IL-18-treated cells had almost identical cell distributions among the clusters indicating that IL-15+IL-18 temporarily alters NK cells phenotype with NK cells returning to a phenotype in line with vehicle control-treated cells within a week of culture after thawing, maintaining NK cell heterogeneity.

### Cryopreserved IL-15|IL-18-Pretreated and NK Cells are Equipotent Compared to Non-Cryopreserved NK Cells In Vivo

A key measure of the cryostability of a cell therapy product is a comparison of non-cryopreserved versus cryopreserved cells in vivo. To examine this, a disseminated xenograft lymphoma model using B2M KO Raji cells was established, which are sensitive to NK cell-mediated killing independent of NK cell donor KIR haplotype. NSG mice bearing disseminated B2M KO Raji cell lymphomas were treated with non-cryopreserved or cryopreserved NK cells IV with or without IL-15+IL-18 pretreatment, and without any post-thaw recovery of NK cells in IL-2 media or dose adjustment (FIG. 31A). IL-15+IL-18-pretreated cryopreserved NK cells achieved significantly improved tumor control, expansion in the peripheral blood, and survival compared to untreated cryopreserved NK cells, and in fact were equipotent as compared to non-cryopreserved untreated NK cells (FIGS. 31B, 31C, 31D), indicating that the full potency of NK cells can be preserved by IL-15+IL-18 treatment prior to cryopreservation.

### Example 2: Development of a Membrane-Bound IL-18 Fusion Protein for Use in NK Cell Culture

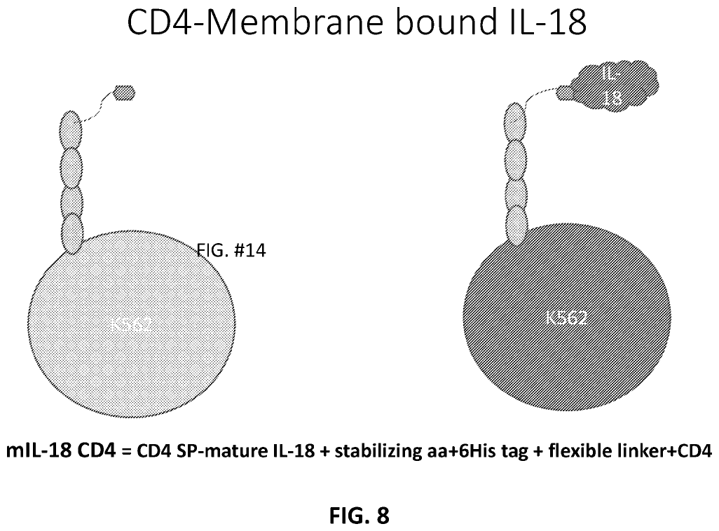

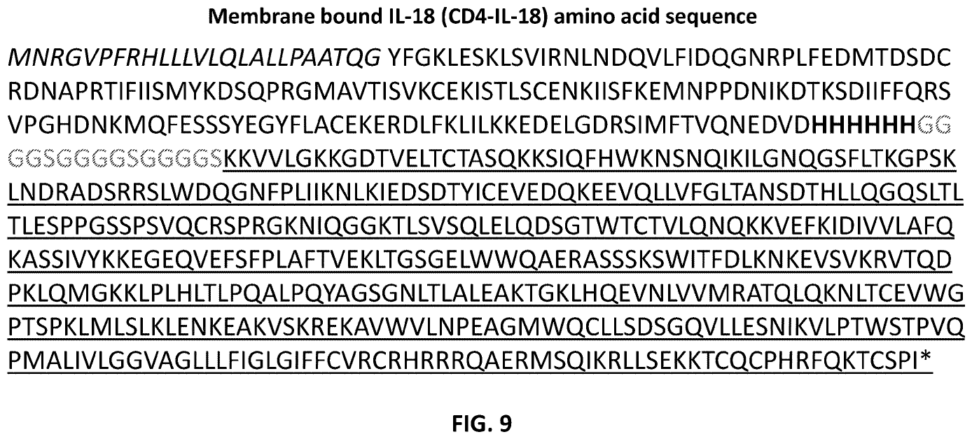

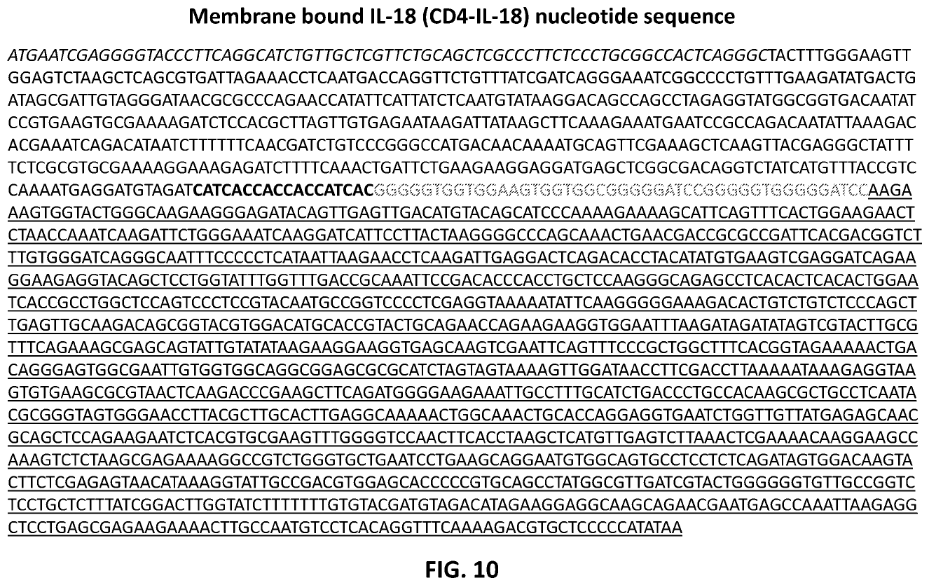

Previous studies presented herein used exogenous, recombinant IL-18 added to NK cell cultures. In order to economize and optimize IL-18 pre-treatment of NK cells, a fusion protein was created which fuses the IL-18 protein to a human CD4 extracellular domain via a flexible linker. This fusion protein would then be expressed by feeder cells, which would eliminate the need for the addition of exogenous cytokine to culture media. FIG. 8 is a diagram showing the structure of membrane bound mature IL-18 (CD4-IL-18). A human CD4 transmembrane extracellular domain was used. Mature IL-18 was tag with 6His tag then attached to CD4 with a Flexible linker 3(G4S), as well as several amino acid changes which stabilized the protein. As a control, a construct was made without the IL-18. FIG. 9 displays the amino acid sequence of the membrane-bound IL-18 fusion protein. Italics=CD4 signal peptide, Plain=mature form of IL-18, Bold=6His tag, Gray=G4S linker and underline=CD4 receptor. FIG. 10 shows the nucleotide sequence encoding the fusion protein. These sequences are also listed in the Table 1.

TABLE 1
IL18-CD4 Fusion Protein Sequences

| SEQ | | |
|---|---|---|
| ID | | |
| NO: | Name: | Sequence: |
| 1. | CD4-IL-18 | MNRGVPFRHLLLVLQLALLPAATQGYFGKLESKLSVIRN |
| | fusion protein, | LNDQVLFIDQGNRPLFEDMTDSDCRDNAPRTIFIISMYKD |
| | AA | SQPRGMAVTISVKCEKISTLSCENKIISFKEMNPPDNIKDT |
| | | KSDIIFFQRSVPGHDNKMQFESSSYEGYFLACEKERDLFK |
| | | LILKKEDELGDRSIMFTVQNEDVDHHHHHHGGGGSGGG |
| | | GSGGGGSKKVVLGKKGDTVELTCTASQKKSIQFHWKNS |
| | | NQIKILGNQGSFLTKGPSKLNDRADSRRSLWDQGNFPLII |
| | | KNLKIEDSDTYICEVEDQKEEVQLLVFGLTANSDTHLLQ |
| | | GQSLTLTLESPPGSSPSVQCRSPRGKNIQGGKTLSVSQLEL |
| | | QDSGTWTCTVLQNQKKVEFKIDIVVLAFQKASSIVYKKE |
| | | GEQVEFSFPLAFTVEKLTGSGELWWQAERASSSKSWITF |
| | | DLKNKEVSVKRVTQDPKLQMGKKLPLHLTLPQALPQYA |
| | | GSGNLTLALEAKTGKLHQEVNLVVMRATQLQKNLTCEV |
| | | WGPTSPKLMLSLKLENKEAKVSKREKAVWVLNPEAGM |
| | | WQCLLSDSGQVLLESNIKVLPTWSTPVQPMALIVLGGVA |
| | | GLLLFIGLGIFFCVRCRHRRRQAERMSQIKRLLSEKKTCQ |
| | | CPHRFQKTCSPI* |
| 2. | CD4 Signal | MNRGVPFRHLLLVLQLALLPAATQG |
| | Peptide, AA | |
| 3. | Mature IL-18, | YFGKLESKLSVIRNLNDQVLFIDQGNRPLFEDMTDSDCR |
| | AA | DNAPRTIFIISMYKDSQPRGMAVTISVKCEKISTLSCENKII |
| | | SFKEMNPPDNIKDTKSDIIFFQRSVPGHDNKMQFESSSYE |
| | | GYFLACEKERDLFKLILKKEDELGDRSIMFTVQNEDVD |
| 4. | 6x His Tag | HHHHHH |
| 5. | G4S Linker | GGGGSGGGGSGGGGS |
| 6. | CD4 | KKVVLGKKGDTVELTCTASQKKSIQFHWKNSNQIKILGN |
| | Receptor, AA | QGSFLTKGPSKLNDRADSRRSLWDQGNFPLIIKNLKIEDS |
| | | DTYICEVEDQKEEVQLLVFGLTANSDTHLLQGQSLTLTL |
| | | ESPPGSSPSVQCRSPRGKNIQGGKTLSVSQLELQDSGTWT |
| | | CTVLQNQKKVEFKIDIVVLAFQKASSIVYKKEGEQVEFSF |
| | | PLAFTVEKLTGSGELWWQAERASSSKSWITFDLKNKEVS |
| | | VKRVTQDPKLQMGKKLPLHLTLPQALPQYAGSGNLTLA |
| | | LEAKTGKLHQEVNLVVMRATQLQKNLTCEVWGPTSPKL |
| | | MLSLKLENKEAKVSKREKAVWVLNPEAGMWQCLLSDS |
| | | GQVLLESNIKVLPTWSTPVQPMALIVLGGVAGLLLFIGLG |
| | | IFFCVRCRHRRRQAERMSQIKRLLSEKKTCQCPHRFQKTC |
| | | SP |
| 7. | CD4-IL-18 | ATGAATCGAGGGGTACCCTTCAGGCATCTGTTGCTCGT |
| | fusion protein, | TCTGCAGCTCGCCCTTCTCCCTGCGGCCACTCAGGGCT |
| | DNA | ACTTTGGGAAGTTGGAGTCTAAGCTCAGCGTGATTAG |
| | | AAACCTCAATGACCAGGTTCTGTTTATCGATCAGGGAA |
| | | ATCGGCCCCTGTTTGAAGATATGACTGATAGCGATTGT |
| | | AGGGATAACGCGCCCAGAACCATATTCATTATCTCAAT |
| | | GTATAAGGACAGCCAGCCTAGAGGTATGGCGGTGACA |
| | | ATATCCGTGAAGTGCGAAAAGATCTCCACGCTTAGTTG |
| | | TGAGAATAAGATTATAAGCTTCAAAGAAATGAATCCG |
| | | CCAGACAATATTAAAGACACGAAATCAGACATAATCT |
| | | TTTTTCAACGATCTGTCCCGGGCCATGACAACAAAATG |
| | | CAGTTCGAAAGCTCAAGTTACGAGGGCTATTTTCTCGC |
| | | GTGCGAAAAGGAAAGAGATCTTTTCAAACTGATTCTG |
| | | AAGAAGGAGGATGAGCTCGGCGACAGGTCTATCATGT |
| | | TTACCGTCCAAAATGAGGATGTAGATCATCACCACCA |
| | | CCATCACGGGGGTGGTGGAAGTGGTGGCGGGGGATCC |
| | | GGGGGTGGGGGATCCAAGAAAGTGGTACTGGGCAAG |
| | | AAGGGAGATACAGTTGAGTTGACATGTACAGCATCCC |
| | | AAAAGAAAAGCATTCAGTTTCACTGGAAGAACTCTAA |
| | | CCAAATCAAGATTCTGGGAAATCAAGGATCATTCCTTA |
| | | CTAAGGGGCCCAGCAAACTGAACGACCGCGCCGATTC |
| | | ACGACGGTCTTTGTGGGATCAGGGCAATTTCCCCCTCA |
| | | TAATTAAGAACCTCAAGATTGAGGACTCAGACACCTA |
| | | CATATGTGAAGTCGAGGATCAGAAGGAAGAGGTACAG |
| | | CTCCTGGTATTTGGTTTGACCGCAAATTCCGACACCCA |
| | | CCTGCTCCAAGGGCAGAGCCTCACACTCACACTGGAA |
| | | TCACCGCCTGGCTCCAGTCCCTCCGTACAATGCCGGTC |
| | | CCCTCGAGGTAAAAATATTCAAGGGGGAAAGACACTG |
| | | TCTGTCTCCCAGCTTGAGTTGCAAGACAGCGGTACGTG |
| | | GACATGCACCGTACTGCAGAACCAGAAGAAGGTGGAA |
| | | TTTAAGATAGATATAGTCGTACTTGCGTTTCAGAAAGC |
| | | GAGCAGTATTGTATATAAGAAGGAAGGTGAGCAAGTC |
| | | GAATTCAGTTTCCCGCTGGCTTTCACGGTAGAAAAACT |
| | | GACAGGGAGTGGCGAATTGTGGTGGCAGGCGGAGCGC |
| | | GCATCTAGTAGTAAAAGTTGGATAACCTTCGACCTTAA |
| | | AAATAAAGAGGTAAGTGTGAAGCGCGTAACTCAAGAC |
| | | CCGAAGCTTCAGATGGGGAAGAAATTGCCTTTGCATCT |
| | | GACCCTGCCACAAGCGCTGCCTCAATACGCGGGTAGT |
| | | GGGAACCTTACGCTTGCACTTGAGGCAAAAACTGGCA |
| | | AACTGCACCAGGAGGTGAATCTGGTTGTTATGAGAGC |
| | | AACGCAGCTCCAGAAGAATCTCACGTGCGAAGTTTGG |
| | | GGTCCAACTTCACCTAAGCTCATGTTGAGTCTTAAACT |
| | | CGAAAACAAGGAAGCCAAAGTCTCTAAGCGAGAAAA |
| | | GGCCGTCTGGGTGCTGAATCCTGAAGCAGGAATGTGG |
| | | CAGTGCCTCCTCTCAGATAGTGGACAAGTACTTCTCGA |
| | | GAGTAACATAAAGGTATTGCCGACGTGGAGCACCCCC |
| | | GTGCAGCCTATGGCGTTGATCGTACTGGGGGGTGTTGC |
| | | CGGTCTCCTGCTCTTTATCGGACTTGGTATCTTTTTTTG |
| | | TGTACGATGTAGACATAGAAGGAGGCAAGCAGAACGA |
| | | ATGAGCCAAATTAAGAGGCTCCTGAGCGAGAAGAAAA |
| | | CTTGCCAATGTCCTCACAGGTTTCAAAAGACGTGCTCC |
| | | CCCATATAA |
| 8. | CD4 Signal | ATGAATCGAGGGGTACCCTTCAGGCATCTGTTGCTCGT |
| | Peptide, DNA | TCTGCAGCTCGCCCTTCTCCCTGCGGCCACTCAGGGC |
| 9. | Mature IL-18, | ACTTTGGGAAGTTGGAGTCTAAGCTCAGCGTGATTAG |
| | DNA | AAACCTCAATGACCAGGTTCTGTTTATCGATCAGGGAA |
| | | ATCGGCCCCTGTTTGAAGATATGACTGATAGCGATTGT |
| | | AGGGATAACGCGCCCAGAACCATATTCATTATCTCAAT |
| | | GTATAAGGACAGCCAGCCTAGAGGTATGGCGGTGACA |
| | | ATATCCGTGAAGTGCGAAAAGATCTCCACGCTTAGTTG |
| | | TGAGAATAAGATTATAAGCTTCAAAGAAATGAATCCG |
| | | CCAGACAATATTAAAGACACGAAATCAGACATAATCT |
| | | TTTTTCAACGATCTGTCCCGGGCCATGACAACAAAATG |
| | | CAGTTCGAAAGCTCAAGTTACGAGGGCTATTTTCTCGC |
| | | GTGCGAAAAGGAAAGAGATCTTTTCAAACTGATTCTG |
| | | AAGAAGGAGGATGAGCTCGGCGACAGGTCTATCATGT |
| | | TTACCGTCCAAAATGAGGATGTAGAT |
| 10. | 6x HIS Tag, | CATCACCACCACCATCAC |
| | DNA | |
| 11. | G4S Linker, | GGGGGTGGTGGAAGTGGTGGCGGGGGATCCGGGGGTG |
| | DNA | GGGGATCC |
| 12. | CD4 | AAGAAAGTGGTACTGGGCAAGAAGGGAGATACAGTTG |
| | Receptor, | AGTTGACATGTACAGCATCCCAAAAGAAAAGCATTCA |
| | DNA | GTTTCACTGGAAGAACTCTAACCAAATCAAGATTCTGG |
| | | GAAATCAAGGATCATTCCTTACTAAGGGGCCCAGCAA |
| | | ACTGAACGACCGCGCCGATTCACGACGGTCTTTGTGG |
| | | GATCAGGGCAATTTCCCCCTCATAATTAAGAACCTCAA |
| | | GATTGAGGACTCAGACACCTACATATGTGAAGTCGAG |
| | | GATCAGAAGGAAGAGGTACAGCTCCTGGTATTTGGTT |
| | | TGACCGCAAATTCCGACACCCACCTGCTCCAAGGGCA |
| | | GAGCCTCACACTCACACTGGAATCACCGCCTGGCTCCA |
| | | GTCCCTCCGTACAATGCCGGTCCCCTCGAGGTAAAAAT |
| | | ATTCAAGGGGGAAAGACACTGTCTGTCTCCCAGCTTG |
| | | AGTTGCAAGACAGCGGTACGTGGACATGCACCGTACT |
| | | GCAGAACCAGAAGAAGGTGGAATTTAAGATAGATATA |
| | | GTCGTACTTGCGTTTCAGAAAGCGAGCAGTATTGTATA |
| | | TAAGAAGGAAGGTGAGCAAGTCGAATTCAGTTTCCCG |
| | | CTGGCTTTCACGGTAGAAAAACTGACAGGGAGTGGCG |
| | | AATTGTGGTGGCAGGCGGAGCGCGCATCTAGTAGTAA |
| | | AAGTTGGATAACCTTCGACCTTAAAAATAAAGAGGTA |
| | | AGTGTGAAGCGCGTAACTCAAGACCCGAAGCTTCAGA |
| | | TGGGGAAGAAATTGCCTTTGCATCTGACCCTGCCACAA |
| | | GCGCTGCCTCAATACGCGGGTAGTGGGAACCTTACGC |
| | | TTGCACTTGAGGCAAAAACTGGCAAACTGCACCAGGA |
| | | GGTGAATCTGGTTGTTATGAGAGCAACGCAGCTCCAG |
| | | AAGAATCTCACGTGCGAAGTTTGGGGTCCAACTTCACC |
| | | TAAGCTCATGTTGAGTCTTAAACTCGAAAACAAGGAA |
| | | GCCAAAGTCTCTAAGCGAGAAAAGGCCGTCTGGGTGC |
| | | TGAATCCTGAAGCAGGAATGTGGCAGTGCCTCCTCTCA |
| | | GATAGTGGACAAGTACTTCTCGAGAGTAACATAAAGG |
| | | TATTGCCGACGTGGAGCACCCCCGTGCAGCCTATGGC |
| | | GTTGATCGTACTGGGGGGTGTTGCCGGTCTCCTGCTCT |
| | | TTATCGGACTTGGTATCTTTTTTTGTGTACGATGTAGAC |
| | | ATAGAAGGAGGCAAGCAGAACGAATGAGCCAAATTA |
| | | AGAGGCTCCTGAGCGAGAAGAAAACTTGCCAATGTCC |
| | | TCACAGGTTTCAAAAGACGTGCTCCCCCATATAA |
| 13 | CD4-IL-18 | MNRGVPFRHLLLVLQLALLPAATQGYFGKLESKLSVIRN |
| | fusion protein, | LNDQVLFIDQGNRPLFEDMTDSDCRDNAPRTIFIISMYKD |
| | AA, no His | SQPRGMAVTISVKCEKISTLSCENKIISFKEMNPPDNIKDT |
| | tag | KSDIIFFQRSVPGHDNKMQFESSSYEGYFLACEKERDLFK |
| | | LILKKEDELGDRSIMFTVQNEDVDGGGGSGGGGSGGGGS |
| | | KKVVLGKKGDTVELTCTASQKKSIQFHWKNSNQIKILGN |
| | | QGSFLTKGPSKLNDRADSRRSLWDQGNFPLIIKNLKIEDS |
| | | DTYICEVEDQKEEVQLLVFGLTANSDTHLLQGQSLTLTL |
| | | ESPPGSSPSVQCRSPRGKNIQGGKTLSVSQLELQDSGTWT |
| | | CTVLQNQKKVEFKIDIVVLAFQKASSIVYKKEGEQVEFSF |
| | | PLAFTVEKLTGSGELWWQAERASSSKSWITFDLKNKEVS |
| | | VKRVTQDPKLQMGKKLPLHLTLPQALPQYAGSGNLTLA |
| | | LEAKTGKLHQEVNLVVMRATQLQKNLTCEVWGPTSPKL |
| | | MLSLKLENKEAKVSKREKAVWVLNPEAGMWQCLLSDS |
| | | GQVLLESNIKVLPTWSTPVQPMALIVLGGVAGLLLFIGLG |
| | | IFFCVRCRHRRRQAERMSQIKRLLSEKKTCQCPHRFQKTC |
| | | SPI* |

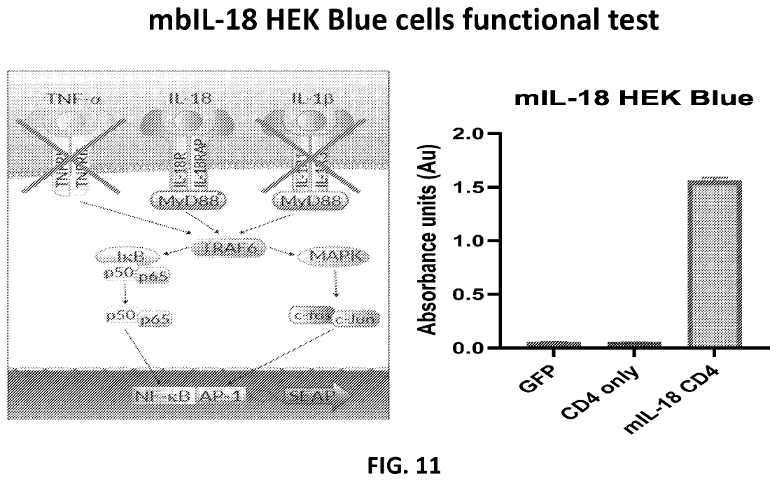

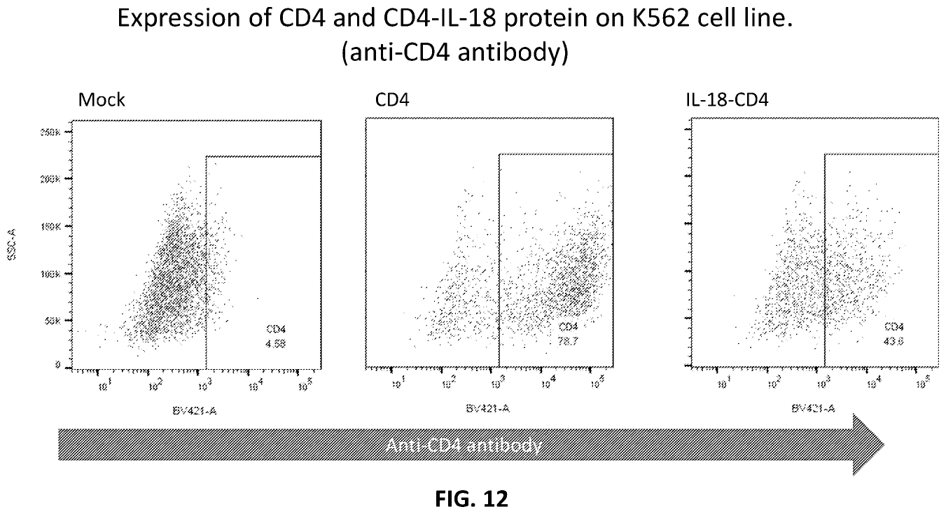

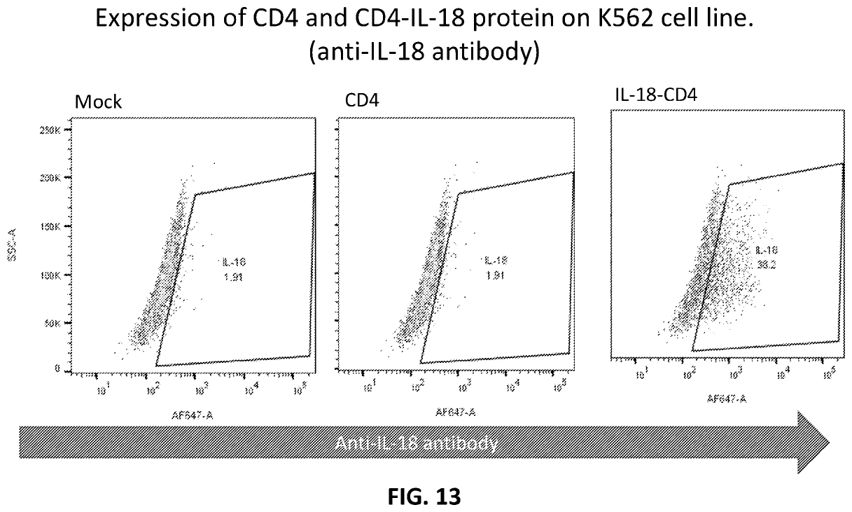

In order to test the CD4-IL-18 fusion construct, IL-18 Reporter HEK 293 Cells were used. These cells transcribe and secrete alkaline phosphatase (SEAP) upon encountering IL-18, the SEAP gene product can then be detected in culture medium using a chemiluminescent assay. IL-18-reporter cells HEK cells were electroplated with plasmids encoding CD4 or the CD4-IL-18 fusion protein. Significant increases in SEAP were seen with CD4-IL-18 but not with CD4 plasmid demonstrating that the CD4-IL-18 construct successfully activates the IL-18 pathway. See FIG. 11. Surface expression of then verified by flow cytometry by staining for both CD4 (FIG. 12) and IL-18 (FIG. 13).

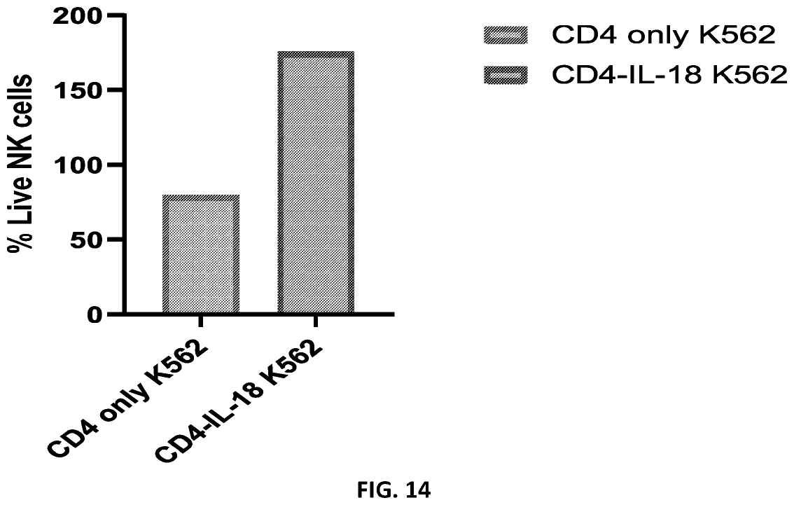

The CD4-IL-18 fusion HEK 293 cells were then used as feeder cells to assess their ability to encourage NK cell growth and expansion, as well as protect NK cells from death and dysfunction following cryopreservation. NK cells were co-cultured with irradiated K562 cells expressing either CD4 or membrane bound CD-IL-18 for 24 hours before cryopreservation. NK cells cultured with K562 cells expressing CD4-IL-18 co-cultured NK had a better recovery after thawing at 24-hour time point (FIG. 14).

### Example 3: In Vivo Use of IL-18 Pre-Treated NK Cells

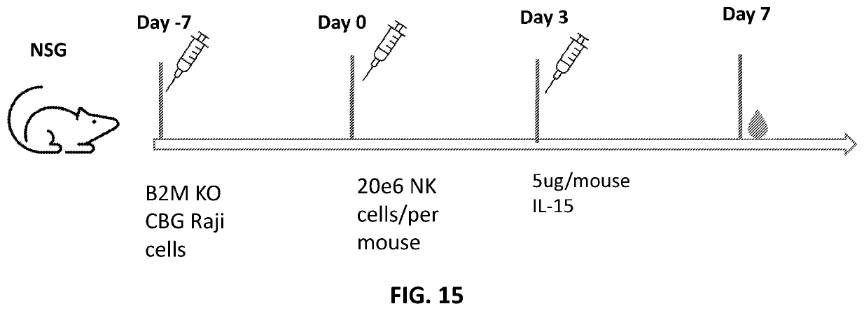

Previous studies disclosed herein demonstrated that the pre-treatment of NK cells with either soluble recombinant IL-18 or irradiated feeder cells expressing the CD4-IL-18 fusion construct maintained NK cell viability and function in vitro after cryopreservation. A series of studies was then conducted in order to verify if IL-18 pre-treated NK cells were functional in vivo. FIG. 15 is a diagram showing a mouse tumor model with NK cell treatment. NSG mice were first injected with 750,000 B2M KO CBG positive Raji cells I.V (tail injection) per mouse. Seven days after tumor injection, mice were then injected with 20×10⁶ NK cells per mouse. For groups receiving IL-18+IL-15 combination. Mice were injected with 5 ug per mouse IL-15 three days after NK injection. Mice were then imaged every 3 days starting at day 0 after NK cell injection. Mice were bled at day 7 after NK injection.

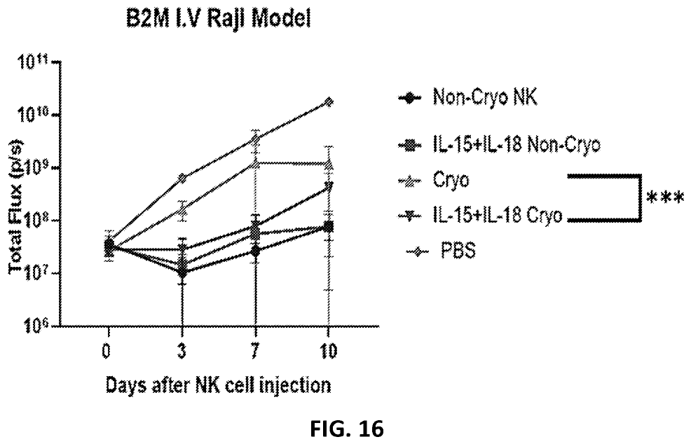

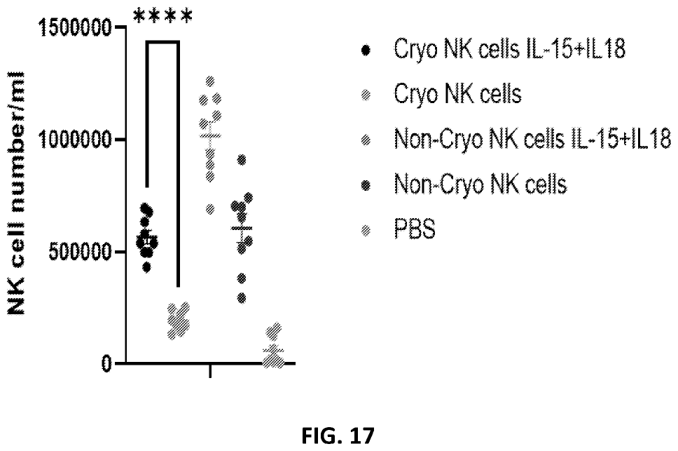

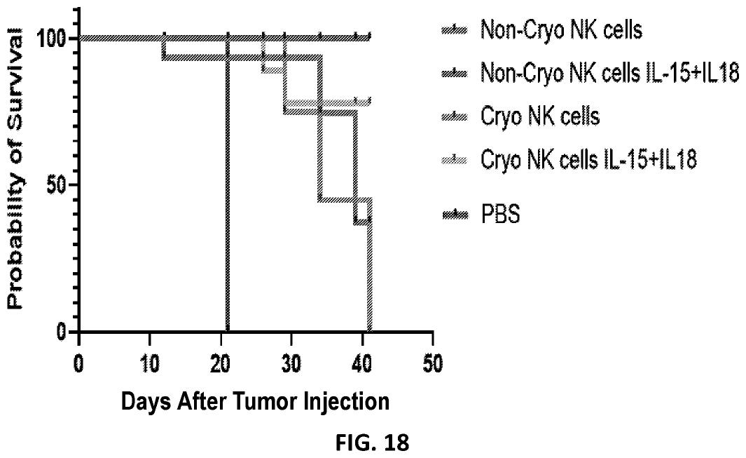

FIG. 16 illustrates the results of the studies using this Raji tumor model treated with fresh and cryopreserved NK cells. Total flux was measured at day 0, 3, 7 and 10 after NK injection. Results found that NK cells treated with IL-15+IL-18 controlled the tumor significantly better than untreated cells. one-way ANOVA p=0.0001 at day 3 and 7 after NK cell injection. Peripheral blood was collected from mice via cheek vein puncture and NK cell count in the blood was measured using TruCount tubes (BD) and flow cytometry. FIG. 17 shows that mice treated with IL-15+IL-18 NK cells had significantly more NK cell in the blood. Mice were then followed for up to 40 days post tumor injection in order to observe overall survival. FIG. 18 shows that mice treated with IL-15+IL-18 NK cells survived longer than mice treated with untreated NK cells.

In total, and without wishing to be bound by theory, these results demonstrate that pre-treatment of NK cells with either IL-18 or feeder cells engineered to express a CD4-IL-18 fusion protein construct prior to cryopreservation significantly improves NK cell viability and function, especially when combined with IL-15 either before cryopreservation or post infusion. These data further suggest the use of IL-18 pre-treatment in the clinical preparation of NK cells in order to maximize the efficacy of this treatment strategy.

### Enumerated Embodiments

The following enumerated embodiments are provided, the numbering of which is not to be construed as designating levels of importance.

Embodiment 1 provides a method for cryopreserving an isolated immune cell or precursor cell thereof, said method comprising:
a. pre-treating the expanded immune cells or precursor cells thereof for at least 24 hours with IL-18 at a concentration between 1.0 and 500 ng/ml;
d. resuspending the pre-treated immune cell or precursor cell thereof in a cryoprotective medium and freezing the cell at −80° C.; and
e. storing the cryopreserved cell in liquid nitrogen (N2);
wherein the method maintains viability and cytotoxic function of the cryopreserved immune cell or precursor cell thereof after thawing.

Embodiment 2 provides the method of claim 1, wherein the isolated immune cell or precursor cell thereof is cultured or expanded for at least 7 days prior to cryopreservation.

Embodiment 3 provides the method of claim 1, further comprising pre-treating the expanded immune cells or precursors thereof with an additional cytokine.

Embodiment 4 provides the method of claim 2, wherein the additional cytokine is IL-15, wherein the IL-15 is used at a concentration between 1.0 and 500 ng/ml.

Embodiment 5 provides the method of claim 1, wherein the immune cell or precursor thereof is a natural killer (NK) cell or NK cell precursor cell.

Embodiment 6 provides the method of claim 5, wherein the NK cell is isolated from a source selected from the group consisting of a patient, a healthy adult donor, cord blood, CD34+ cord blood stem cells, an induced pluripotent stem cell (iPSC), and an NK cell line.

Embodiment 7 provides the method of claim 1, wherein the immune cell is a T cell or T cell precursor cell.

Embodiment 8 provides the method of claim 1, wherein the subject is a mammal.

Embodiment 9 provides the method of claim 1, wherein the subject is a human.

Embodiment 10 provides a fusion protein comprising a first protein domain and a second protein domain separated by a linker, wherein the first protein domain comprises IL-18 peptide and the second protein domain comprises a human CD4 receptor.

Embodiment 11 provides the fusion protein of claim 10, wherein the IL-18 peptide is a mature IL-18 peptide.

Embodiment 12 provides the fusion protein of claim 10, wherein the IL-18 peptide comprises the amino acid sequence set forth in SEQ ID NO: 3.

Embodiment 13 provides the fusion protein of claim 10, wherein the CD4 receptor domain comprises the amino acid sequence set forth in SEQ ID NO: 6.

Embodiment 14 provides the fusion protein of claim 10, further comprising a CD4 signal peptide.

Embodiment 15 provides the fusion protein of claim 14, wherein the CD4 signal peptide comprises the amino acid sequence set forth in SEQ IN NO: 2.

Embodiment 16 provides the fusion protein of claim 10, wherein the linker is a G4S linker.

Embodiment 17 provides the fusion protein of claim 10, wherein the linker comprises the amino acid sequence set forth in SEQ ID NO: 5.

Embodiment 18 provides the fusion protein of claim 10, wherein the fusion protein comprises the amino acid sequence set forth in SEQ ID NOs: 1 or 13.

Embodiment 19 provides an isolated nucleic acid encoding a fusion protein comprising a first protein domain and a second protein domain separated by a linker, wherein the first protein domain comprises IL-18 peptide and the second protein domain comprises a human CD4 receptor.

Embodiment 20 provides the isolated nucleic acid of claim 19, wherein the IL-18 peptide is a mature IL-18 peptide.

Embodiment 21 provides the isolated nucleic acid of claim 19, wherein the IL-18 peptide is encoded by a nucleic acid sequence set forth in SEQ ID NO: 9.

Embodiment 22 provides the isolated nucleic acid of claim 19, wherein the CD4 receptor domain is encoded by a nucleic acid sequence set forth in SEQ ID NO: 12.

Embodiment 23 provides the isolated nucleic acid of claim 19, further comprising a nucleic acid sequence encoding a CD4 signal peptide.

Embodiment 24 provides the isolated nucleic acid of claim 23, wherein the nucleic acid sequence encoding a CD4 signal peptide comprises the nucleic acid sequence set forth in SEQ IN NO: 8.

Embodiment 25 provides the isolated nucleic acid of claim 19, wherein the linker is a G4S linker.

Embodiment 26 provides the isolated nucleic acid of claim 25, wherein the linker is encoded by a nucleic acid sequence set forth in SEQ ID NO: 11.

Embodiment 27 provides the isolated nucleic acid of claim 19, wherein the fusion protein is encoded by a nucleic acid sequence set forth in SEQ ID NO: 7.

Embodiment 28 provides a vector comprising the isolated nucleic acid of any one of claims 19-27.

Embodiment 29 provides the vector of claim 26, wherein the vector is an expression vector.

Embodiment 30 provides the vector of claim 26, wherein the vector is selected from the group consisting of a DNA vector, an RNA vector, a plasmid, a lentiviral vector, an adenoviral vector, an adeno-associated vector, and a retroviral vector.

Embodiment 31 provides a host cell comprising the isolated nucleic acid of any one of claims 19-27 or the vector of any one of claims 28-30.

Embodiment 32 provides the host cell of claim 31, wherein the host cell is of eukaryotic origin.

Embodiment 33 provides the host cell of claim 31 or 32, wherein the host cell is of mammalian origin.

Embodiment 34 provides the host cell of any one of claims 31-33, wherein the host cell is selected from the group consisting of a K562 cell, a HEK293, and an EBV-transformed cell.

Embodiment 35 provides a method for producing a composition comprising an isolated immune cell or precursor cell thereof, said method comprising:
a. pre-treating the isolated immune cell or precursor cell thereof for at least 24 hours with IL-18 at a concentration between 1.0 and 500 ng/ml;
d. resuspending the pre-treated immune cell or precursor cell thereof in a cryoprotective medium and freezing the cell at −80° C.;
e. storing the cryopreserved cell in liquid nitrogen (N2); and
f. thawing cryopreserved cell and resuspending it in a pharmaceutically acceptable excipient, carrier, or diluent.

Embodiment 36 provides the method of claim 25, further comprising culturing or expanding the isolated immune cell or precursor thereof for at least 7 days.

Embodiment 37 provides the method of claim 35, further comprising pre-treating the expanded immune cells or precursors thereof with an additional cytokine.

Embodiment 38 provides the method of claim 37, wherein the additional cytokine is IL-15, wherein the IL-15 is used at a concentration between 1.0 and 500 ng/ml.

Embodiment 39 provides the method of claim 34, wherein the immune cell or precursor thereof is a natural killer (NK) cell or NK cell precursor cell.

Embodiment 40 provides the method of claim 39, wherein the NK cell is isolated from a source selected from the group consisting of a patient, a healthy adult donor, cord blood, CD34+ cord blood stem cells, an induced pluripotent stem cell (iPSC), and an NK cell line.

Embodiment 41 provides the method of claim 35, wherein the immune cell is a T cell or T cell precursor cell.

Embodiment 42 provides the method of claim 35, wherein the subject is a mammal.

Embodiment 43 provides the method of claim 35, wherein the subject is a human.

Embodiment 44 provides a method for producing a composition comprising an isolated immune cell or precursor cell thereof, said method comprising:
a. co-culturing the isolated immune cell or precursor cell thereof with a composition comprising an IL-18-CD4 fusion protein;
d. resuspending the pre-treated immune cell or precursor cell thereof in a cryoprotective medium and freezing the cell at −80° C.;
e. storing the cryopreserved cell in liquid nitrogen (N2); and
f. thawing cryopreserved cell and resuspending it in a pharmaceutically acceptable excipient, carrier, or diluent.

Embodiment 45 provides the method of claim 44, wherein the composition comprises an irradiated feeder cell.

Embodiment 46 provides the method of claim 44, wherein the composition comprises a cell-derived vesicle that comprises the IL-18-CD4 fusion protein.

Embodiment 47 provides the method of claim 44, wherein the isolated immune cell or precursor cell thereof is cultured or expanded for at least 7 days prior to cryopreservation.

Embodiment 48 provides the method of claim 44, wherein the fusion protein comprises a first protein domain and a second protein domain separated by a linker, wherein the first protein domain comprises IL-18 peptide and the second protein domain comprises a human CD4 receptor.

Embodiment 49 provides the method of claim 48, wherein the IL-18 peptide comprises the amino acid sequence set forth in SEQ ID NO: 3.

Embodiment 50 provides the method of claim 48, wherein the CD4 receptor domain comprises the amino acid sequence set forth in SEQ ID NO: 6.

Embodiment 51 provides the method of claim 44, wherein the fusion protein further comprises a CD4 signal peptide.

Embodiment 52 provides the method of claim 51, wherein the CD4 signal peptide comprises the amino acid sequence set forth in SEQ IN NO: 2.

Embodiment 53 provides the method of claim 48, wherein the linker is a G4S linker.

Embodiment 54 provides the method of claim 53, wherein the linker comprises the amino acid sequence set forth in SEQ ID NO: 5.

Embodiment 55 provides the method of claim 44, wherein the fusion protein comprises the amino acid sequence set forth in SEQ ID NOS: 1 OR 13.

Embodiment 56 provides the method of claim 44, further comprising pre-treating the expanded immune cells or precursors thereof with an additional cytokine.

Embodiment 57 provides the method of claim 56, wherein the additional cytokine is IL-15, wherein the IL-15 is used at a concentration between 1.0 and 500 ng/ml.

Embodiment 58 provides the method of claim 44, wherein the immune cell or precursor thereof is a natural killer (NK) cell or NK cell precursor cell.

Embodiment 59 provides the method of claim 58, wherein the NK cell is isolated from a source selected from the group consisting of a patient, a healthy adult donor, cord blood, CD34+ cord blood stem cells, an induced pluripotent stem cell (iPSC), and an NK cell line.

Embodiment 60 provides the method of claim 44, wherein the immune cell is a T cell or T cell precursor cell.

Embodiment 61 provides the method of claim 44, wherein the subject is a mammal.

Embodiment 62 provides the method of claim 44, wherein the subject is a human.

Embodiment 63 provides a method for treating a disease in a subject in need thereof, comprising administering to the subject an effective amount of the composition of claim 35, thereby treating the disease.

Embodiment 64 provides the method of claim 63, wherein the disease is a cancer.

Embodiment 65 provides the method of claim 63, further comprising administering to the subject an additional therapeutic agent.

Embodiment 66 provides the method of claim 65, wherein the additional therapeutic agent is a cytokine.

Embodiment 67 provides the method of claim 66, wherein the cytokine is IL-15.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method for cryopreserving an isolated immune cell or precursor cell thereof, said method comprising:
a. pre-treating the isolated immune cell or precursor cells thereof for at least 24 hours with IL-18 at a concentration between 1.0 and 500 ng/ml;
b. resuspending the pre-treated immune cell or precursor cell thereof in a cryoprotective medium and freezing the cell at −80° C.; and
c. storing the cryopreserved cell in liquid nitrogen (N2);
wherein the method maintains viability and cytotoxic function of the cryopreserved immune cell or precursor cell thereof after thawing.

2. The method of claim 1, wherein the isolated immune cell or precursor cell thereof is cultured or expanded for at least 7 days prior to cryopreservation.

3. The method of claim 1, further comprising pre-treating the expanded immune cells or precursors thereof with an additional cytokine.

4. The method of claim 1, wherein the immune cell or precursor thereof is a natural killer (NK) cell or NK cell precursor cell.

5. The method of claim 4, wherein the NK cell is isolated from a source selected from the group consisting of a patient, a healthy adult donor, cord blood, CD34+ cord blood stem cells, an induced pluripotent stem cell (iPSC), and an NK cell line.

6. The method of claim 3, wherein the additional cytokine is IL-15, wherein the IL-15 is used at a concentration between 1.0 and 500 ng/ml.

7. The method of claim 1, wherein the immune cell is a T cell or T cell precursor cell.

8-9. (canceled)

10. A fusion protein comprising a first protein domain and a second protein domain separated by a linker, wherein the first protein domain comprises IL-18 peptide and the second protein domain comprises a human CD4 receptor.

11. The fusion protein of claim 10, wherein the IL-18 peptide is a mature IL-18 peptide.

12. The fusion protein of claim 10, wherein the IL-18 peptide comprises the amino acid sequence set forth in SEQ ID NO: 3.

13. The fusion protein of claim 10, wherein the CD4 receptor domain comprises the amino acid sequence set forth in SEQ ID NO: 6.

14. The fusion protein of claim 10, further comprising a CD4 signal peptide.

15. The fusion protein of claim 14, wherein the CD4 signal peptide comprises the amino acid sequence set forth in SEQ IN NO: 2.

16. The fusion protein of claim 10, wherein the linker is a G4S linker.

17. The fusion protein of claim 10, wherein the linker comprises the amino acid sequence set forth in SEQ ID NO: 5.

18. The fusion protein of claim 10, wherein the fusion protein comprises the amino acid sequence set forth in SEQ ID NOs: 1 OR 13.

19-43. (canceled)

44. A method for producing a composition comprising an isolated immune cell or precursor cell thereof, said method comprising:
a. co-culturing the isolated immune cell or precursor cell thereof with a composition comprising an IL-18-CD4 fusion protein;
b. resuspending the pre-treated immune cell or precursor cell thereof in a cryoprotective medium and freezing the cell at −80° C.;
c. storing the cryopreserved cell in liquid nitrogen (N2); and
d. thawing cryopreserved cell and resuspending it in a pharmaceutically acceptable excipient, carrier, or diluent.

45. The method of claim 44, wherein the composition comprises an irradiated feeder cell.

46. The method of claim 44, wherein the composition comprises a cell-derived vesicle that comprises the IL-18-CD4 fusion protein.

47. The method of claim 44, wherein the isolated immune cell or precursor cell thereof is cultured or expanded for at least 7 days prior to treatment.

48-67. (canceled)
